(19) United States
(12) Patent Application Publication
(10) Pub. No.: US 2023/0183815 A1
(43) Pub. Date: Jun. 15, 2023

(54) DETECTION OF COLORECTAL NEOPLASMS

(71) Applicant: Universal Diagnostics, S.L., Seville (ES)

(72) Inventors: Marko Bitenc, Ljubljana (SI); Kristi Kruusmaa, Ljubljana (SI); Juan Martinez-Barea, Seville (ES); Christian Hense, Seville (ES); Pol Sola de los Santos, Seville (ES); Pol Canal Noguer, Seville (ES); Marko Chersicola, Ljubljana (SI); Primoz Knap, Ljubljana (SI)

(21) Appl. No.: 18/088,298

(22) Filed: Dec. 23, 2022

Related U.S. Application Data
(62) Division of application No. 17/027,110, filed on Sep. 21, 2020, now abandoned.
(60) Provisional application No. 63/046,510, filed on Jun. 30, 2020.

(51) Int. Cl. C12Q 1/6886

(52) U.S. Cl. CPC C12Q 1/6886; C12Q 2600/112; C12Q 2600/154

(57) ABSTRACT

The present disclosure provides, among other things, methods for colorectal neoplasm detection (e.g., screening) and compositions related thereto. In various embodiments, the present disclosure provides methods for adenoma and/or early stage colorectal cancer detection (e.g., screening) and compositions related thereto. In various embodiments, the present disclosure provides methods for screening that include analysis of methylation status of one or more methylation biomarkers, and compositions related thereto. In various embodiments, the present disclosure provides methods for detection (e.g., screening) that include detecting (e.g., screening) methylation status of one or more methylation biomarkers in cfDNA, e.g., in ctDNA. In various embodiments, the present disclosure provides methods for screening that include detecting (e.g., screening) methylation status of one or more methylation biomarkers in cfDNA, e.g., in ctDNA, using MSRE-qPCR and/or using massively parallel sequencing (e.g., next-generation sequencing).

### CROSS-REFERENCE

This application claims priority to and benefit of U.S. Provisional Application No. 63/046,510 filed on Jun. 30, 2020. The contents of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Sep. 17, 2020, is named 2011722_0058_SL.txt and is 27,203 bytes in size.

### BACKGROUND

Screening for colorectal neoplasms such as colorectal cancer is a critical component of cancer prevention, diagnosis, and treatment. Colorectal cancer (CRC) has been identified, according to some reports, as the third most common type of cancer and the second most frequent cause of cancer mortality in the world. According to some reports, there are over 1.8 million new cases of colorectal cancer per year and about 881,000 deaths from colorectal cancer, accounting for about 1 in 10 cancer deaths. Regular colorectal cancer screening is recommended, particular for individuals over age 45. Moreover, incidence of colorectal cancer in individuals below 50 has increased over time. Statistics suggest that current colorectal cancer screening techniques are insufficient.

Furthermore, detecting colon cancers at an early stage would result in decreased mortality rates. Detection and removal of precursors of colon cancer, including but not limited to, colonic polyps with advanced features, will reduce the incidence of CRC as these polyps are believed to represent the greatest risk of malignant progression. Removal of advanced colonic polyps would mitigate the risk of cancer initiation. Generally, about 9-16% of the asymptomatic patients aged 50 and older present with advanced adenoma findings.

Accordingly, there exists a need for methods, compositions, and systems that can provide for classification and/or diagnosis of colorectal neoplasms. In particular, there is a need for diagnosis and/or classification of colorectal neoplasms at an early stage.

### SUMMARY

The present disclosure provides, among other things, methods for detecting (e.g., screening for) colorectal neoplasms and compositions related thereto. In various embodiments, the present disclosure provides methods for classification of subjects having and/or not having premalignant and/or malignant colorectal neoplasms including, but not limited to, advanced adenomas, polyposis, colorectal cancer (e.g., stage 0, I, II, III, or an undifferentiated stage), and/or various combinations thereof. In various embodiments, the present disclosure provides methods for colorectal neoplasm screening that include determination of methylation status (e.g., the number, frequency, or pattern of methylation) at one or more methylation sites found within one or more markers within a sample (e.g., a blood sample, a blood product sample, a stool sample, a colorectal tissue sample) from a subject (e.g., a human subject), and compositions related thereto. For example, markers may include a methylation locus, e.g., a differentially methylated region (DMR) of deoxyribonucleic acid (DNA) of a human subject. In various embodiments, the present disclosure provides methods for classifying a subject as having and/or not having advanced adenoma, polyposis, colorectal cancer, and/or any combination thereof that includes determining methylation status for each of one or more methylation loci in cfDNA (cell free DNA), e.g., in ctDNA (circulating tumor DNA). In various embodiments, the present disclosure provides methods for colorectal neoplasm screening that include determining a methylation status for each of one or more methylation loci in cfDNA, e.g., in ctDNA, using, for example, massive parallel sequencing (e.g., next generation sequencing), e.g., sequencing-by-synthesis, real-time (e.g., single-molecule) sequencing, bead emulsion sequencing, nanopore sequencing, quantitative polymerase chain reaction (qPCR) (e.g., methylation sensitive restriction enzyme quantitative polymerase chain reaction, MSRE-qPCR). Various compositions and methods provided herein provide sensitivity and specificity sufficient for clinical application in screening for conditions, including but not limited to, advanced adenoma, polyposis and/or early stage colorectal cancer. Various compositions and methods provided herein are useful in advanced adenoma, polyposis and/or colorectal cancer screening by analysis of an accessible tissue sample of a subject, e.g., a tissue sample that is blood or a blood component (e.g., cfDNA, e.g., ctDNA), or stool.

In one aspect, the invention is directed to a method of detecting a colorectal neoplasm in a human subject, the method comprising: determining a methylation status of each of one or more markers identified in a sample obtained from the subject, and determining whether the subject has a colorectal neoplasm based at least in part on the determined methylation status of each of the one or more markers, wherein each of the one or more markers is a methylation locus comprising at least a portion of (e.g., at least 20% of) a differentially methylated region (DMR) selected from the DMRs of Table 1 (e.g., corresponding to SEQ ID NOs. 1-54) [e.g., wherein the methylation locus comprises at least 20% of the DMR and wherein the portion of the methylation locus that overlaps with the DMR has at least 98% similarity with the overlapping portion of the DMR].

In certain embodiments, detecting the colorectal neoplasm comprises a member selected from the group consisting of (i) classifying the subject as having advanced adenoma, (ii) classifying the subject as having polyposis, (iii) classifying the subject as having colorectal cancer (e.g., stage 0, I, II, III or undifferentiated stage), (iv) classifying the subject as having at least one of the conditions advanced adenoma, polyposis, and colorectal cancer, either with or without identifying which of those conditions the subject has, and (v) classifying the subject as having at least one of the conditions advanced adenoma and colorectal cancer, either with or without identifying which of those conditions the subject has.

In certain embodiments, the sample is or comprises a blood sample, a blood product sample, a stool sample, a colorectal tissue sample.

In certain embodiments, the method comprises determining a methylation status of at least a portion of [e.g., at least 20% of] each of one or more (one, two, three, or all four) of the following DMRs: SLC6A1 '689 [chr3:10993689-10993900] (SEQ ID NO: 9); F13A1 '205 [chr6:6320205-6320663] (SEQ ID NO: 31); BARHL1 '614 [chr9:132579614-132579683] (SEQ ID NO: 41); and [chr19:20052466-20053193] (SEQ ID NO: 53).

In certain embodiments, the method comprises determining a methylation status of a portion of [e.g., at least 20% of] each of one or more (one, two, three, or all four) of the following DMRs: CSMD2 '443 [chr1:34165443-34165675] (SEQ ID NO:1); SLC6A1 '689 [chr3:10993689-10993900] (SEQ ID NO: 9); [chr6:27670532-27670614] (SEQ ID NO: 33); and [chr19:20052466-20053193] (SEQ ID NO: 53).

In certain embodiments, the method comprises determining the methylation status of each of the one or more markers using next generation sequencing (NGS). In certain embodiments, the method comprises using one or more oligonucleotide capture baits that enrich for a target region to capture one or more corresponding methylation locus/loci.

In certain embodiments, the one or more marker(s) is or comprises at least one (e.g., at least 2, at least 3, at least 4, or more) CpG dinucleotide.

In certain embodiments, the step of determining the methylation status further comprises determining a relative amount (e.g., a percentage) of methylated and unmethylated CpGs and/or determining a read-based pathological methylation pattern.

In certain embodiments, methylation status is determined using methylation sensitive restriction enzyme quantitative polymerase chain reaction (MSRE-qPCR).

In another aspect, the invention is directed to a method of detecting a colorectal neoplasm in a human subject, the method comprising: determining a methylation status for each of one or more of the following, in deoxyribonucleic acid (DNA) of a human subject: (i) a methylation locus within gene SLC6A1; (ii) a methylation locus within gene F13A1; and (iii) a methylation locus within gene BARHL1; and diagnosing colorectal neoplasm in the human subject based at least on said determined methylation status(es).

In certain embodiments, detecting the colorectal neoplasm comprises a member selected from the group consisting of (i) classifying the subject as having advanced adenoma, (ii) classifying the subject as having polyposis, (iii) classifying the subject as having colorectal cancer, (iv) classifying the subject as having at least one of the conditions advanced adenoma, polyposis, and colorectal cancer, either with or without identifying which of those conditions the subject has, and (v) classifying the subject as having at least one of the conditions advanced adenoma and colorectal cancer, either with or without identifying which of those conditions the subject has.

In certain embodiments, the method comprises determining a methylation status for a methylation locus within gene SLC6A1, wherein the methylation locus within gene SLC6A1 comprises at least a portion of (e.g., at least 20% of) SLC6A1 '689 [chr3:10993689-10993900] (SEQ ID NO: 9) [wherein the methylation locus within gene SLC6A1 comprises at least 20% of SLC6A1 '689 and wherein the portion of the methylation locus that overlaps with SLC6A1 has at least 98% similarity with the overlapping portion of SLC6A1 '689].

In certain embodiments, the method comprises determining a methylation status for a methylation locus within gene F13A1, wherein the methylation locus within gene F13A1 comprises at least a portion of (e.g., at least 20% of) F13A1 '205 [chr6:6320205-6320663] (SEQ ID NO: 31) [wherein the methylation locus within gene F13A1 comprises at least 20% of F13A1 '205 and wherein the portion of the methylation locus that overlaps with F13A1 has at least 98% similarity with the overlapping portion of F13A1 '205].

In certain embodiments, the method comprises determining a methylation status for a methylation locus within gene BARHL1, wherein the methylation locus within gene BARHL1 comprises at least a portion of (e.g., at least 20% of) BARHL1 '614 [chr9:132579614-132579683] (SEQ ID NO: 41) [wherein the methylation locus within gene BARHL1 comprises at least 20% of BARHL1 '614 and wherein the portion of the methylation locus that overlaps with BARHL1 has at least 98% similarity with the overlapping portion of BARHL1 '614].

In certain embodiments, the method further comprises determining a methylation status for a methylation locus comprising at least a portion of chr19:20052466-20053193 (SEQ ID NO: 53) in deoxyribonucleic acid (DNA) of the human subject, and wherein the diagnosing step comprises diagnosing colorectal neoplasm in the human subject based at least on the determined methylation status for the methylation locus comprising said at least portion of chr19:20052466-20053193 (SEQ ID NO: 53) [e.g., wherein the methylation locus comprises at least 20% of the chr19:20052466-20053193 and wherein the portion of the methylation locus that overlaps with chr19:20052466-20053193 has at least 98% similarity with the overlapping portion of chr19:20052466-20053193].

In certain embodiments, the DNA is isolated from blood or plasma of the human subject.

In certain embodiments, the step of determining the methylation status further comprises determining a relative amount (e.g., a percentage) of methylated and unmethylated CpGs and/or determining a read-based pathological methylation pattern.

In certain embodiments, methylation status is determined using methylation sensitive restriction enzyme quantitative polymerase chain reaction (MSRE-qPCR).

In certain embodiments, the method comprises determining a methylation status for a methylation locus within gene SLC6A1, wherein the methylation locus of SLC6A1 is or comprises at least one (e.g., at least 2, at least 3, at least 4, or more) CpG dinucleotide.

In certain embodiments, the method comprises determining a methylation status for a methylation locus within gene F13A1, comprising determining a methylation status for a methylation locus within gene F13A1, wherein the methylation locus of F13A1 is or comprises at least one (e.g., at least 2, at least 3, at least 4, or more) CpG dinucleotide.

In certain embodiments, the method comprises determining a methylation status for a methylation locus within gene BARHL1, wherein the methylation locus of BARHL1 is or comprises at least one (e.g., at least 2, at least 3, at least 4, or more) CpG dinucleotide.

In another aspect, the invention is directed to a method of detecting a colorectal neoplasm in a human subject, the method comprising: determining a methylation status for each of one or both of the following, in deoxyribonucleic acid (DNA) of a human subject: (i) a methylation locus within gene CSMD2; and (ii) a methylation locus within gene SLC6A1; and diagnosing the colorectal neoplasm in the human subject based at least on said determined methylation status(es).

In certain embodiments, detecting the colorectal neoplasm comprises a member selected from the group consisting of (i) classifying the subject as having advanced adenoma, (ii) classifying the subject as having polyposis, (iii) classifying the subject as having colorectal cancer, (iv) classifying the subject as having at least one of the conditions advanced adenoma, polyposis, and colorectal cancer, either with or without identifying which of those conditions the subject has, and (v) classifying the subject as having at least one of the conditions advanced adenoma and colorectal cancer, either with or without identifying which of those conditions the subject has.

In certain embodiments, the method comprises determining a methylation status for a methylation locus within gene CSMD2, wherein the methylation locus of gene CSMD2 comprises at least one (e.g., at least 2, at least 3, at least 4, or more) CpG dinucleotide.

In certain embodiments, the method comprises determining a methylation status for a methylation locus within gene SLC6A1, wherein the methylation locus of gene SLC6A1 comprises at least one (e.g., at least 2, at least 3, at least 4, or more) CpG dinucleotide.

In certain embodiments, the method comprises determining a methylation status for a methylation locus within gene CSMD2, wherein the methylation locus within gene CSMD2 comprises at least a portion of (e.g., at least 20% of) CSMD2 '443 [chr1:34165443-34165675] (SEQ ID NO: 1) [wherein the methylation locus within gene CSMD2 comprises at least 20% of CSMD2 '443 and wherein the portion of the methylation locus that overlaps with CSMD2 has at least 98% similarity with the overlapping portion of CSMD2 '443].

In certain embodiments, the method comprises determining a methylation status for a methylation locus within gene SLCA1, wherein the methylation locus within gene SLC6A1 comprises at least a portion of (e.g., at least 20% of) SLC6A1 '689 [chr3:10993689-10993900] (SEQ ID NO: 9) [wherein the methylation locus within gene SLCA1 comprises at least 20% of SLC6A1 '689 and wherein the portion of the methylation locus that overlaps with SLCA1 has at least 98% similarity with the overlapping portion of SLC6A1 '689].

In certain embodiments, the method comprises determining a methylation status for a methylation locus comprising at least a portion of chr6:27670532-27670614 (SEQ ID NO: 33) in deoxyribonucleic acid (DNA) of the human subject, and wherein the diagnosing step comprises diagnosing colorectal neoplasm in the human subject based at least on the determined methylation status for the methylation locus comprising said at least portion of chr6:27670532-27670614 (SEQ ID NO: 33) [wherein the methylation locus comprises at least 20% of chr6:27670532-27670614 and wherein the portion of the methylation locus that overlaps with chr6:27670532-27670614 has at least 98% similarity with the overlapping portion of chr6:27670532-27670614].

In certain embodiments, the method further comprises determining a methylation status for a methylation locus comprising at least a portion of chr19:20052466-20053193 (SEQ ID NO: 53) in deoxyribonucleic acid (DNA) of the human subject, and wherein the diagnosing step comprises diagnosing colorectal neoplasm in the human subject based at least on the determined methylation status for the methylation locus comprising said at least portion of chr19:20052466-20053193 (SEQ ID NO: 53) [wherein the methylation locus comprises at least 20% of chr19:20052466-20053193 and wherein the portion of the methylation locus that overlaps with chr19:20052466-20053193 has at least 98% similarity with the overlapping portion of chr19:20052466-20053193].

In certain embodiments, the DNA is isolated from blood or plasma of the human subject.

In certain embodiments, the DNA is cell-free DNA of the human subject.

In certain embodiments, methylation status is determined using quantitative polymerase chain reaction (qPCR).

In certain embodiments, methylation status is determined using methylation sensitive restriction enzyme quantitative polymerase chain reaction (MSRE-qPCR).

In certain embodiments, methylation status is determined using massively parallel sequencing.

In certain embodiments, each methylation locus is equal to or less than 5000 bp in length.

In certain embodiments, the method comprises determining the methylation status of each of the one or more markers using next generation sequencing (NGS). In certain embodiments, the method comprises using one or more oligonucleotide capture baits (e.g., biotinylated oligonucleotide probes) that enrich for a target region to capture one or more corresponding methylation locus/loci (e.g., followed by library preparation and sequencing, e.g., wherein the sample is either bisulfate converted or enzymatically converted prior to capture).

In certain embodiments, determining the methylation status further comprises determining a relative amount (e.g., a percentage) of methylated and unmethylated CpGs and/or determining a read-based pathological methylation pattern.

In another aspect, the invention is directed to a kit for use in a method as described herein, the kit comprising one or more oligonucleotide primer pairs (e.g., a forward and reverse primer pair) for amplification of one or more corresponding methylation locus/loci.

In another aspect, the invention is directed to a diagnostic qPCR reaction for detection (e.g., screening) of colorectal cancer in a method as described herein, the diagnostic qPCR reaction including: (i) human DNA, (ii) a polymerase; and (iii) one or more oligonucleotide primer pairs for amplification of one or more corresponding methylation locus/loci, and, optionally, at least one methylation sensitive restriction enzyme.

In certain embodiments, each of the one or more corresponding methylation locus/loci comprise at least one methylation sensitive restriction enzyme (MSRE) cleavage site (e.g., at least 2, 3, 4, 5, 6, 7, 8, 9, 10, 11, 12, 13, 14, or 15 MSRE cleavage sites).

In another aspect, the invention is directed to a kit for use in a method described herein, the kit comprising one or more oligonucleotide capture baits (e.g., one or more biotinylated oligonucleotide probes) for capturing one or more corresponding methylation locus/loci (e.g., for hybridizing to the region/regions of interest).

In certain embodiments, the one or more oligonucleotide capture baits comprise one or more biotinylated oligonucleotide probes.

In various aspects, methods as described herein may further comprise treatment of a cancer (e.g., colorectal cancer, advanced adenoma) based on, at least, the methylation status of one or more methylation loci.

In various aspects, methods and compositions of the present invention can be used in combination with biomarkers known in the art, e.g., as disclosed in U.S. Pat. Nos. 10,006,925 and 63,011,970, which are herein incorporated by reference in their entirety.

### Definitions

A or An: The articles “a” and “an” are used herein to refer to one or to more than one (i.e., to at least one) of the grammatical object of the article. By way of example, “an element” refers to one element or more than one element.

About: The term “about”, when used herein in reference to a value, refers to a value that is similar, in context, to the referenced value. In general, those skilled in the art, familiar with the context, will appreciate the relevant degree of variance encompassed by “about” in that context. For example, in some embodiments, e.g., as set forth herein, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or with a fraction of a percent, of the referred value.

Advanced Adenoma: As used herein, the term “advanced adenoma” typically refers to refer to cells that exhibit first indications of relatively abnormal, uncontrolled, and/or autonomous growth but are not yet classified as cancerous alterations. In the context of colon tissue, “advanced adenoma” refers to neoplastic growth that shows signs of high grade dysplasia, and/or size that is >=10 mm, and/or villious histological type, and/or serrated histological type with any type of dysplasia.

Administration: As used herein, the term “administration” typically refers to the administration of a composition to a subject or system, for example to achieve delivery of an agent that is, is included in, or is otherwise delivered by, the composition.

Agent: As used herein, the term “agent” refers to an entity (e.g., for example, a small molecule, peptide, polypeptide, nucleic acid, lipid, polysaccharide, complex, combination, mixture, system, or phenomenon such as heat, electric current, electric field, magnetic force, magnetic field, etc.).

Amelioration: As used herein, the term “amelioration” refers to the prevention, reduction, palliation, or improvement of a state of a subject. Amelioration includes, but does not require, complete recovery or complete prevention of a disease, disorder or condition.

Amplicon or amplicon molecule: As used herein, the term “amplicon” or “amplicon molecule” refers to a nucleic acid molecule generated by transcription from a template nucleic acid molecule, or a nucleic acid molecule having a sequence complementary thereto, or a double-stranded nucleic acid including any such nucleic acid molecule. Transcription can be initiated from a primer.

Amplification: As used herein, the term “amplification” refers to the use of a template nucleic acid molecule in combination with various reagents to generate further nucleic acid molecules from the template nucleic acid molecule, which further nucleic acid molecules may be identical to or similar to (e.g., at least 70% identical, e.g., at least 75%, 80%, 85%, 90%, 91%, 92%, 93%, 94%, 95%, 96%, 97%, 98%, 99%, or 100% identical to) a segment of the template nucleic acid molecule and/or a sequence complementary thereto.

Amplification reaction mixture: As used herein, the terms “amplification reaction mixture” or “amplification reaction” refer to a template nucleic acid molecule together with reagents sufficient for amplification of the template nucleic acid molecule.

Biological Sample: As used herein, the term “biological sample” typically refers to a sample obtained or derived from a biological source (e.g., a tissue or organism or cell culture) of interest, as described herein. In some embodiments, e.g., as set forth herein, a biological source is or includes an organism, such as an animal or human. In some embodiments, e.g., as set forth herein, a biological sample is or include biological tissue or fluid. In some embodiments, e.g., as set forth herein, a biological sample can be or include cells, tissue, or bodily fluid. In some embodiments, e.g., as set forth herein, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or broncheoalveolar lavages, aspirates, scrapings, bone marrow. In some embodiments, e.g., as set forth herein, a biological sample is or includes cells obtained from a single subject or from a plurality of subjects. A sample can be a “primary sample” obtained directly from a biological source, or can be a “processed sample.” A biological sample can also be referred to as a “sample.”

Biomarker: As used herein, the term “biomarker,” consistent with its use in the art, refers to a to an entity whose presence, level, or form, correlates with a particular biological event or state of interest, so that it is considered to be a “marker” of that event or state. Those of skill in the art will appreciate, for instance, in the context of a DNA biomarker, that a biomarker can be or include a locus (such as one or more methylation loci) and/or the status of a locus (e.g., the status of one or more methylation loci). To give but a few examples of biomarkers, in some embodiments, e.g., as set forth herein, a biomarker can be or include a marker for a particular disease, disorder or condition, or can be a marker for qualitative of quantitative probability that a particular disease, disorder or condition can develop, occur, or reoccur, e.g., in a subject. In some embodiments, e.g., as set forth herein, a biomarker can be or include a marker for a particular therapeutic outcome, or qualitative of quantitative probability thereof. Thus, in various embodiments, e.g., as set forth herein, a biomarker can be predictive, prognostic, and/or diagnostic, of the relevant biological event or state of interest. A biomarker can be an entity of any chemical class. For example, in some embodiments, e.g., as set forth herein, a biomarker can be or include a nucleic acid, a polypeptide, a lipid, a carbohydrate, a small molecule, an inorganic agent (e.g., a metal or ion), or a combination thereof. In some embodiments, e.g., as set forth herein, a biomarker is a cell surface marker. In some embodiments, e.g., as set forth herein, a biomarker is intracellular. In some embodiments, e.g., as set forth herein, a biomarker is found outside of cells (e.g., is secreted or is otherwise generated or present outside of cells, e.g., in a body fluid such as blood, urine, tears, saliva, cerebrospinal fluid, and the like). In some embodiments, e.g., as set forth herein, a biomarker is methylation status of a methylation locus. In some instances, e.g., as set forth herein, a biomarker may be referred to as a “marker.”

To give but one example of a biomarker, in some embodiments e.g., as set forth herein, the term refers to expression of a product encoded by a gene, expression of which is characteristic of a particular tumor, tumor subclass, stage of tumor, etc. Alternatively or additionally, in some embodiments, e.g., as set forth herein, presence or level of a particular marker can correlate with activity (or activity level) of a particular signaling pathway, for example, of a signaling pathway the activity of which is characteristic of a particular class of tumors.

Those of skill in the art will appreciate that a biomarker may be individually determinative of a particular biological event or state of interest, or may represent or contribute to a determination of the statistical probability of a particular biological event or state of interest. Those of skill in the art will appreciate that markers may differ in their specificity and/or sensitivity as related to a particular biological event or state of interest.

Blood component: As used herein, the term “blood component” refers to any component of whole blood, including red blood cells, white blood cells, plasma, platelets, endothelial cells, mesothelial cells, epithelial cells, and cell-free DNA. Blood components also include the components of plasma, including proteins, metabolites, lipids, nucleic acids, and carbohydrates, and any other cells that can be present in blood, e.g., due to pregnancy, organ transplant, infection, injury, or disease.

Cancer: As used herein, the terms “cancer,” “malignancy,” “neoplasm,” “tumor,” and “carcinoma,” are used interchangeably to refer to a disease, disorder, or condition in which cells exhibit or exhibited relatively abnormal, uncontrolled, and/or autonomous growth, so that they display or displayed an abnormally elevated proliferation rate and/or aberrant growth phenotype. In some embodiments, e.g., as set forth herein, a cancer can include one or more tumors. In some embodiments e.g., as set forth herein, a cancer can be or include cells that are precancerous (e.g., benign), malignant, pre-metastatic, metastatic, and/or non-metastatic. In some embodiments e.g., as set forth herein, a cancer can be or include a solid tumor. In some embodiments e.g., as set forth herein, a cancer can be or include a hematologic tumor. In general, examples of different types of cancers known in the art include, for example, colorectal cancer, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Chemotherapeutic agent: As used herein, the term “chemotherapeutic agent,” consistent with its use in the art, refers to one or more agents known, or having characteristics known to, treat or contribute to the treatment of cancer. In particular, chemotherapeutic agents include pro-apoptotic, cytostatic, and/or cytotoxic agents. In some embodiments e.g., as set forth herein, a chemotherapeutic agent can be or include alkylating agents, anthracyclines, cytoskeletal disruptors (e.g., microtubule targeting moieties such as taxanes, maytansine, and analogs thereof, of), epothilones, histone deacetylase inhibitors HDACs), topoisomerase inhibitors (e.g., inhibitors of topoisomerase I and/or topoisomerase II), kinase inhibitors, nucleotide analogs or nucleotide precursor analogs, peptide antibiotics, platinum-based agents, retinoids, vinca alkaloids, and/or analogs that share a relevant anti-proliferative activity. In some particular embodiments e.g., as set forth herein, a chemotherapeutic agent can be or include of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g., DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, or a combination thereof. In some embodiments e.g., as set forth herein, a chemotherapeutic agent can be utilized in the context of an antibody-drug conjugate. In some embodiments e.g., as set forth herein, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine. In some embodiments e.g., as set forth herein, a chemotherapeutic agent can be or comprise of farnesyl-thiosalicylic acid (FTS), 4-(4-Chloro-2-methylphenoxy)-N-hydroxybutanamide (CMH), estradiol (E2), tetramethoxystilbene (TMS), δ-tocatrienol, salinomycin, or curcumin.

Combination therapy: As used herein, the term “combination therapy” refers to administration to a subject of to two or more agents or regimens such that the two or more agents or regimens together treat a disease, condition, or disorder of the subject. In some embodiments, e.g., as set forth herein, the two or more therapeutic agents or regimens can be administered simultaneously, sequentially, or in overlapping dosing regimens. Those of skill in the art will appreciate that combination therapy includes but does not require that the two agents or regimens be administered together in a single composition, nor at the same time.

Comparable: As used herein, the term “comparable” refers to members within sets of two or more conditions, circumstances, agents, entities, populations, etc., that may not be identical to one another but that are sufficiently similar to permit comparison there between, such that one of skill in the art will appreciate that conclusions can reasonably be drawn based on differences or similarities observed. In some embodiments, e.g., as sort forth herein, comparable sets of conditions, circumstances, agents, entities, populations, etc. are typically characterized by a plurality of substantially identical features and zero, one, or a plurality of differing features. Those of ordinary skill in the art will understand, in context, what degree of identity is required to render members of a set comparable. For example, those of ordinary skill in the art will appreciate that members of sets of conditions, circumstances, agents, entities, populations, etc., are comparable to one another when characterized by a sufficient number and type of substantially identical features to warrant a reasonable conclusion that differences observed can be attributed in whole or part to non-identical features thereof.

Detectable moiety: The term “detectable moiety” as used herein refers to any element, molecule, functional group, compound, fragment, or other moiety that is detectable. In some embodiments, e.g., as sort forth herein, a detectable moiety is provided or utilized alone. In some embodiments, e.g., as sort forth herein, a detectable moiety is provided and/or utilized in association with (e.g., joined to) another agent. Examples of detectable moieties include, but are not limited to, various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents, bioluminescent agents, spectrally resolvable inorganic fluorescent semiconductors nanocrystals (i.e., quantum dots), metal nanoparticles, nanoclusters, paramagnetic metal ions, enzymes, colorimetric labels, biotin, dioxigenin, haptens, and proteins for which antisera or monoclonal antibodies are available.

Diagnosis: As used herein, the term “Diagnosis” refers to determining whether, and/or the qualitative of quantitative probability that, a subject has or will develop a disease, disorder, condition, or state. For example, in diagnosis of cancer, diagnosis can include a determination regarding the risk, type, stage, malignancy, or other classification of a cancer. In some instances, e.g., as sort forth herein, a diagnosis can be or include a determination relating to prognosis and/or likely response to one or more general or particular therapeutic agents or regimens.

Diagnostic information: As used herein, the term “diagnostic information” refers to information useful in providing a diagnosis. Diagnostic information can include, without limitation, biomarker status information.

Differentially methylated: As used herein, the term “differentially methylated” describes a methylation site for which the methylation status differs between a first condition and a second condition. A methylation site that is differentially methylated can be referred to as a differentially methylated site. In some instances, e.g., as sort forth herein, a DMR is defined by the amplicon produced by amplification using oligonucleotide primers, e.g., a pair of oligonucleotide primers selected for amplification of the DMR or for amplification of a DNA region of interest present in the amplicon. In some instances, e.g., as sort forth herein, a DMR is defined as a DNA region amplified by a pair of oligonucleotide primers, including the region having the sequence of, or a sequence complementary to, the oligonucleotide primers. In some instances, e.g., as sort forth herein, a DMR is defined as a DNA region amplified by a pair of oligonucleotide primers, excluding the region having the sequence of, or a sequence complementary to, the oligonucleotide primers. As used herein, a specifically provided DMR can be unambiguously identified by the name of an associated gene followed by three digits of a starting position, such that, for example, a DMR starting at position 29921434 of ALK can be identified as ALK '434. As used herein, a specifically provided DMR can be unambiguously identified by the chromosome number followed by the starting and ending positions of a DMR. For example, a DMR identified in Table 1 as having the uid 9_132579614_132579683 may also be identified as chr9:132579614-132579683.

Differentially methylated region: As used herein, the term “differentially methylated region” (DMR) refers to a DNA region that includes one or more differentially methylated sites. A DMR that includes a greater number or frequency of methylated sites under a selected condition of interest, such as a cancerous state, can be referred to as a hypermethylation DMR. A DMR that includes a smaller number or frequency of methylated sites under a selected condition of interest, such as a cancerous state, can be referred to as a hypomethylation DMR. A DMR that is a methylation biomarker for colorectal cancer can be referred to as a colorectal cancer DMR. In some instances, e.g., as set forth herein, a DMR can be a single nucleotide, which single nucleotide is a methylation site. In some instances, e.g., as set forth herein, a DMR has a length of at least 10, at least 15, at least 20, at least 30, at least 50, or at least 75 base pairs. In some instances, e.g., as set forth herein, a DMR has a length of equal to or less than 5000 bp, 4,000 bp, 3,000 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp (e.g., where methylation status is determined using quantitative polymerase chain reaction (qPCR), e.g., methylation sensitive restriction enzyme quantitative polymerase chain reaction (MSRE-qPCR)). In some instances, e.g., as set forth herein, a DMR that is a methylation biomarker for advanced adenoma may also be useful in identification of colorectal cancer.

DNA region: As used herein, “DNA region” refers to any contiguous portion of a larger DNA molecule. Those of skill in the art will be familiar with techniques for determining whether a first DNA region and a second DNA region correspond, based, e.g., on sequence similarity (e.g, sequence identity or homology) of the first and second DNA regions and/or context (e.g., the sequence identity or homology of nucleic acids upstream and/or downstream of the first and second DNA regions).

Except as otherwise specified herein, sequences found in or relating to humans (e.g., that hybridize to human DNA) are found in, based on, and/or derived from the example representative human genome sequence commonly referred to, and known to those of skill in the art, as Homo sapiens (human) genome assembly GRCh38, hg38, and/or Genome Reference Consortium Human Build 38. Those of skill in the art will further appreciate that DNA regions of hg38 can be referred to by a known system including identification of particular nucleotide positions or ranges thereof in accordance with assigned numbering.

Dosing regimen: As used herein, the term “dosing regimen” can refer to a set of one or more same or different unit doses administered to a subject, typically including a plurality of unit doses administration of each of which is separated from administration of the others by a period of time. In various embodiments, e.g., as set forth herein, one or more or all unit doses of a dosing regimen may be the same or can vary (e.g., increase over time, decrease over time, or be adjusted in accordance with the subject and/or with a medical practitioner's determination). In various embodiments, e.g., as set forth herein, one or more or all of the periods of time between each dose may be the same or can vary (e.g., increase over time, decrease over time, or be adjusted in accordance with the subject and/or with a medical practitioner's determination). In some embodiments, e.g., as set forth herein, a given therapeutic agent has a recommended dosing regimen, which can involve one or more doses. Typically, at least one recommended dosing regimen of a marketed drug is known to those of skill in the art. In some embodiments, e.g., as set forth herein, a dosing regimen is correlated with a desired or beneficial outcome when administered across a relevant population (i.e., is a therapeutic dosing regimen).

Downstream: As used herein, the term “downstream” means that a first DNA region is closer, relative to a second DNA region, to the C-terminus of a nucleic acid that includes the first DNA region and the second DNA region.

Gene: As used herein, the term “gene” refers to a single DNA region, e.g., in a chromosome, that includes a coding sequence that encodes a product (e.g., an RNA product and/or a polypeptide product), together with all, some, or none of the DNA sequences that contribute to regulation of the expression of coding sequence. In some embodiments, e.g., as set forth herein, a gene includes one or more non-coding sequences. In some particular embodiments, e.g., as set forth herein, a gene includes exonic and intronic sequences. In some embodiments, e.g., as set forth herein, a gene includes one or more regulatory elements that, for example, can control or impact one or more aspects of gene expression (e.g., cell-type-specific expression, inducible expression, etc.). In some embodiments, e.g., as set forth herein, a gene includes a promoter. In some embodiments, e.g., as set forth herein, a gene includes one or both of a (i) DNA nucleotides extending a predetermined number of nucleotides upstream of the coding sequence and (ii) DNA nucleotides extending a predetermined number of nucleotides downstream of the coding sequence. In various embodiments, e.g., as set forth herein, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb.

Homology: As used herein, the term “homology” refers to the overall relatedness between polymeric molecules, e.g., between nucleic acid molecules (e.g., DNA molecules and/or RNA molecules) and/or between polypeptide molecules. Those of skill in the art will appreciate that homology can be defined, e.g., by a percent identity or by a percent homology (sequence similarity). In some embodiments, e.g., as set forth herein, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, e.g., as set forth herein, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Hybridize: As used herein, “hybridize” refers to the association of a first nucleic acid with a second nucleic acid to form a double-stranded structure, which association occurs through complementary pairing of nucleotides. Those of skill in the art will recognize that complementary sequences, among others, can hybridize. In various embodiments, e.g., as set forth herein, hybridization can occur, for example, between nucleotide sequences having at least 70% complementarity, e.g., at least 75%, 80%, 85%, 90%, 91%, 92%, 93%, 94%, 95%, 96%, 97%, 98%, 99%, or 100% complementarity. Those of skill in the art will further appreciate that whether hybridization of a first nucleic acid and a second nucleic acid does or does not occur can dependence upon various reaction conditions. Conditions under which hybridization can occur are known in the art.

Hypomethylation: As used herein, the term “hypomethylation” refers to the state of a methylation locus having at least one fewer methylated nucleotides in a state of interest as compared to a reference state (e.g., at least one fewer methylated nucleotides in colorectal cancer than in a healthy control).

Hypermethylation: As used herein, the term “hypermethylation” refers to the state of a methylation locus having at least one more methylated nucleotide in a state of interest as compared to a reference state (e.g., at least one more methylated nucleotide in colorectal cancer than in a healthy control).

Identity, identical: As used herein, the terms “identity” and “identical” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. Calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences and, optionally, taking into account the number of gaps and the length of each gap, which may need to be introduced for optimal alignment of the two sequences. The comparison of sequences and determination of percent identity between two sequences can be accomplished using a computational algorithm, such as BLAST (basic local alignment search tool).

“Improved,” “increased,” or “reduced”: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.

Methylation: As used herein, the term “methylation” includes methylation at any of (i) C5 position of cytosine; (ii) N4 position of cytosine; and (iii) the N6 position of adenine. Methylation also includes (iv) other types of nucleotide methylation. A nucleotide that is methylated can be referred to as a “methylated nucleotide” or “methylated nucleotide base.” In certain embodiments, e.g., as set forth herein, methylation specifically refers to methylation of cytosine residues. In some instances, methylation specifically refers to methylation of cytosine residues present in CpG sites.

Methylation assay: As used herein, the term “methylation assay” refers to any technique that can be used to determine the methylation status of a methylation locus.

Methylation biomarker: As used herein, the term “methylation biomarker” refers to a biomarker that is or includes at least one methylation locus and/or the methylation status of at least one methylation locus, e.g., a hypermethylated locus. In particular, a methylation biomarker is a biomarker characterized by a change between a first state and a second state (e.g., between a cancerous state and a non-cancerous state) in methylation status of one or more nucleic acid loci.

Methylation locus: As used herein, the term “methylation locus” refers to a DNA region that includes at least one differentially methylated region. A methylation locus that includes a greater number or frequency of methylated sites under a selected condition of interest, such as a cancerous state, can be referred to as a hypermethylated locus. A methylation locus that includes a smaller number or frequency of methylated sites under a selected condition of interest, such as a cancerous state, can be referred to as a hypomethylated locus. In some instances, e.g., as set forth herein, a methylation locus has a length of at least 10, at least 15, at least 20, at least 30, at least 50, or at least 75 base pairs. In some instances, e.g., as set forth herein, a methylation locus has a length of less than 5000 bp, 4,000 bp, 3,000 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp (e.g., where methylation status is determined using quantitative polymerase chain reaction (qPCR), e.g., methylation sensitive restriction enzyme quantitative polymerase chain reaction (MSRE-qPCR)).

Methylation site: As used herein, a methylation site refers to a nucleotide or nucleotide position that is methylated in at least one condition. In its methylated state, a methylation site can be referred to as a methylated site.

Methylation status: As used herein, “methylation status,” “methylation state,” or “methylation profile” refer to the number, frequency, or pattern of methylation at methylation sites within a methylation locus. Accordingly, a change in methylation status between a first state and a second state can be or include an increase in the number, frequency, or pattern of methylated sites, or can be or include a decrease in the number, frequency, or pattern of methylated sites. In various instances, a change in methylation status in a change in methylation value.

Methylation value: As used herein, the term “methylation value” refers to a numerical representation of a methylation status, e.g., in the form of number that represents the frequency or ratio of methylation of a methylation locus. In some instances, e.g., as set forth herein, a methylation value can be generated by a method that includes quantifying the amount of intact nucleic acid present in a sample following restriction digestion of the sample with a methylation dependent restriction enzyme. In some instances, e.g., as set forth herein, a methylation value can be generated by a method that includes comparing amplification profiles after bisulfite reaction of a sample. In some instances, e.g., as set forth herein, a methylation value can be generated by comparing sequences of bisulfite-treated and untreated nucleic acids. In some instances, e.g., as set forth herein, a methylation value is, includes, or is based on a quantitative PCR result.

Nucleic acid: As used herein, in its broadest sense, the term “nucleic acid” refers to any compound and/or substance that is or can be incorporated into an oligonucleotide chain. In some embodiments e.g., as set forth herein, a nucleic acid is a compound and/or substance that is or can be incorporated into an oligonucleotide chain via a phosphodiester linkage. As will be clear from context, in some embodiments e.g., as set forth herein, the term nucleic acid refers to an individual nucleic acid residue (e.g., a nucleotide and/or nucleoside), and in some embodiments e.g., as set forth herein refers to an polynucleotide chain comprising a plurality of individual nucleic acid residues. A nucleic acid can be or include DNA, RNA, or a combinations thereof. A nucleic acid can include natural nucleic acid residues, nucleic acid analogs, and/or synthetic residues. In some embodiments e.g., as set forth herein, a nucleic acid includes natural nucleotides (e.g., adenosine, thymidine, guanosine, cytidine, uridine, deoxyadenosine, deoxythymidine, deoxy guanosine, and deoxycytidine). In some embodiments e.g., as set forth herein, a nucleic acid is or includes of one or more nucleotide analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof).

In some embodiments e.g., as set forth herein, a nucleic acid has a nucleotide sequence that encodes a functional gene product such as an RNA or protein. In some embodiments e.g., as set forth herein, a nucleic acid includes one or more introns. In some embodiments e.g., as set forth herein, a nucleic acid includes one or more genes. In some embodiments e.g., as set forth herein, nucleic acids are prepared by one or more of isolation from a natural source, enzymatic synthesis by polymerization based on a complementary template (in vivo or in vitro), reproduction in a recombinant cell or system, and chemical synthesis.

In some embodiments e.g., as set forth herein, a nucleic acid analog differs from a nucleic acid in that it does not utilize a phosphodiester backbone. For example, in some embodiments e.g., as set forth herein, a nucleic acid can include one or more peptide nucleic acids, which are known in the art and have peptide bonds instead of phosphodiester bonds in the backbone. Alternatively or additionally, in some embodiments e.g., as set forth herein, a nucleic acid has one or more phosphorothioate and/or 5′-N-phosphoramidite linkages rather than phosphodiester bonds. In some embodiments e.g., as set forth herein, a nucleic acid comprises one or more modified sugars (e.g., 2′-fluororibose, ribose, 2′-deoxyribose, arabinose, and hexose) as compared with those in natural nucleic acids.

In some embodiments, e.g., as set forth herein, a nucleic acid is or includes at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues. In some embodiments, e.g., as set forth herein, a nucleic acid is partly or wholly single stranded, or partly or wholly double stranded.

Nucleic acid detection assay: As used herein, the term “nucleic acid detection assay” refers to any method of determining the nucleotide composition of a nucleic acid of interest. Nucleic acid detection assays include but are not limited to, DNA sequencing methods, polymerase chain reaction-based methods, probe hybridization methods, ligase chain reaction, etc.

Nucleotide: As used herein, the term “nucleotide” refers to a structural component, or building block, of polynucleotides, e.g., of DNA and/or RNA polymers. A nucleotide includes of a base (e.g., adenine, thymine, uracil, guanine, or cytosine) and a molecule of sugar and at least one phosphate group. As used herein, a nucleotide can be a methylated nucleotide or an un-methylated nucleotide. Those of skill in the art will appreciate that nucleic acid terminology, such as, as examples, “locus” or “nucleotide” can refer to both a locus or nucleotide of a single nucleic acid molecule and/or to the cumulative population of loci or nucleotides within a plurality of nucleic acids (e.g., a plurality of nucleic acids in a sample and/or representative of a subject) that are representative of the locus or nucleotide (e.g., having the same identical nucleic acid sequence and/or nucleic acid sequence context, or having a substantially identical nucleic acid sequence and/or nucleic acid context).

Oligonucleotide primer: As used herein, the term oligonucleotide primer, or primer, refers to a nucleic acid molecule used, capable of being used, or for use in, generating amplicons from a template nucleic acid molecule. Under transcription-permissive conditions (e.g., in the presence of nucleotides and a DNA polymerase, and at a suitable temperature and pH), an oligonucleotide primer can provide a point of initiation of transcription from a template to which the oligonucleotide primer hybridizes. Typically, an oligonucleotide primer is a single-stranded nucleic acid between 5 and 200 nucleotides in length. Those of skill in the art will appreciate that optimal primer length for generating amplicons from a template nucleic acid molecule can vary with conditions including temperature parameters, primer composition, and transcription or amplification method. A pair of oligonucleotide primers, as used herein, refers to a set of two oligonucleotide primers that are respectively complementary to a first strand and a second strand of a template double-stranded nucleic acid molecule. First and second members of a pair of oligonucleotide primers may be referred to as a “forward” oligonucleotide primer and a “reverse” oligonucleotide primer, respectively, with respect to a template nucleic acid strand, in that the forward oligonucleotide primer is capable of hybridizing with a nucleic acid strand complementary to the template nucleic acid strand, the reverse oligonucleotide primer is capable of hybridizing with the template nucleic acid strand, and the position of the forward oligonucleotide primer with respect to the template nucleic acid strand is 5′ of the position of the reverse oligonucleotide primer sequence with respect to the template nucleic acid strand. It will be understood by those of skill in the art that the identification of a first and second oligonucleotide primer as forward and reverse oligonucleotide primers, respectively, is arbitrary inasmuch as these identifiers depend upon whether a given nucleic acid strand or its complement is utilized as a template nucleic acid molecule.

Overlapping: The term “overlapping” is used herein in reference to two regions of DNA, each of which contains a sub-sequence that is substantially identical to a sub-sequence of the same length in the other region (e.g., the two regions of DNA have a common sub-sequence). “Substantially identical” means that the two identically-long sub-sequences differ by fewer than a given number of base pairs. In certain instances, e.g., as set forth herein, each sub-sequence has a length of at least 20 base pairs that differ by fewer than 4, 3, 2, or 1 base pairs from each other (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 24 base pairs that differ by fewer than 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 50 base pairs that differ by fewer than 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 100 base pairs that differ by fewer than 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 200 base pairs that differ by fewer than 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 250 base pairs that differ by fewer than 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 300 base pairs that differ by fewer than 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 500 base pairs that differ by fewer than 100, 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 1000 base pairs that differ by fewer than 200, 100, 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, the subsequence of a first region of the two regions of DNA may comprise the entirety of the second region of the two regions of DNA (or vice versa) (e.g., the common sub-sequence may contain the whole of either or both regions). In certain embodiments, where a methylation locus has a sequence that comprises at “least a portion of” a DMR sequence listed herein (e.g., at least 20%, at least 30%, at least 40%, at least 50%, at least 60%, at least 70%, at least 80%, or at least 90% of the DMR sequence), the overlapping portion of the methylation locus has at least 95% similarity, at least 98% similarity, or at least 99% similarity with the overlapping portion of the DMR sequence (e.g., if the overlapping portion is 100 bp, the portion of the methylation locus that overlaps with the portion of the DMR differs by no more than 1 bp, no more than 2 bp, or no more than 5 bp). In certain embodiments, where a methylation locus has a sequence that comprises “at least a portion of” a DMR sequence listed herein, this means the methylation locus has a subsequence in common with the DMR sequence that has a consecutive series of bases that covers at least 20%, at least 30%, at least 40%, at least 50%, at least 60%, at least 70%, at least 80%, or at least 90% of the DMR sequence, e.g., wherein the subsequence in common differs by no more than 1 bp, no more than 2 bp, or no more than 5 bp). In certain embodiments, where a methylation locus has a sequence that comprises “at least a portion of” a DMR sequence listed herein, this means the methylation locus contains at least a portion of (e.g., at least 20%, at least 30%, at least 40%, at least 50%, at least 60%, at least 70%, at least 80%, or at least 90% of) the CpG dinucleotides corresponding to the CpG dinucleotides within the DMR sequence.

Pharmaceutical composition: As used herein, the term “pharmaceutical composition” refers to a composition in which an active agent is formulated together with one or more pharmaceutically acceptable carriers. In some embodiments, e.g., as set forth herein, the active agent is present in a unit dose amount appropriate for administration to a subject, e.g., in a therapeutic regimen that shows a statistically significant probability of achieving a predetermined therapeutic effect when administered to a relevant population. In some embodiments, e.g., as set forth herein, a pharmaceutical composition can be formulated for administration in a particular form (e.g., in a solid form or a liquid form), and/or can be specifically adapted for, for example: oral administration (for example, as a drenche (aqueous or non-aqueous solutions or suspensions), tablet, capsule, bolus, powder, granule, paste, etc., which can be formulated specifically for example for buccal, sublingual, or systemic absorption); parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation and/or modifies bioavailability of an agent, e.g., a pharmaceutical agent. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polyposis syndromes: The terms “polyposis” and “polyposis syndrome”, as used herein, refer to hereditary conditions that include, but are not limited to, familial adenomatous polyposis (FAP), hereditary nonpolyposis colorectal cancer (HNPCC)/Lynch syndrome, Gardner syndrome, Turcot syndrome, MUTYH polyposis, Peutz-Jeghers syndrome, Cowden disease, familial juvenile polyposis, and hyperplastic polyposis. In certain embodiments, polyposis includes serrated polyposis syndrome. Serrated polyposis is classified by a subject having 5 or more serrated polyps proximal to the sigmoid colon with two or more at least 10 mm in size, having a serrated polyp proximal to the sigmoid colon in the context of a family history of serrated polyposis, and/or having 20 or more serrated polyps throughout the colon.

Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.

Probe: As used herein, the term “probe” refers to a single- or double-stranded nucleic acid molecule that is capable of hybridizing with a complementary target and includes a detectable moiety. In certain embodiments, e.g., as set forth herein, a probe is a restriction digest product or is a synthetically produced nucleic acid, e.g., a nucleic acid produced by recombination or amplification. In some instances, e.g., as set forth herein, a probe is a capture probe useful in detection, identification, and/or isolation of a target sequence, such as a gene sequence. In various instances, e.g., as set forth herein, a detectable moiety of probe can be, e.g., an enzyme (e.g., ELISA, as well as enzyme-based histochemical assays), fluorescent moiety, radioactive moiety, or moiety associated with a luminescence signal.

Prognosis: As used herein, the term “prognosis” refers to determining the qualitative of quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.

Prognostic information: As used herein, the term “prognostic information” refers to information useful in providing a prognosis. Prognostic information can include, without limitation, biomarker status information.

Promoter: As used herein, a “promoter” can refer to a DNA regulatory region that directly or indirectly (e.g., through promoter-bound proteins or substances) associates with an RNA polymerase and participates in initiation of transcription of a coding sequence.

Reference: As used herein describes a standard or control relative to which a comparison is performed. For example, in some embodiments, e.g., as set forth herein, an agent, subject, animal, individual, population, sample, sequence, or value of interest is compared with a reference or control agent, subject, animal, individual, population, sample, sequence, or value. In some embodiments, e.g., as set forth herein, a reference or characteristic thereof is tested and/or determined substantially simultaneously with the testing or determination of the characteristic in a sample of interest. In some embodiments, e.g., as set forth herein, a reference is a historical reference, optionally embodied in a tangible medium. Typically, as would be understood by those of skill in the art, a reference is determined or characterized under comparable conditions or circumstances to those under assessment, e.g., with regard to a sample. Those skilled in the art will appreciate when sufficient similarities are present to justify reliance on and/or comparison to a particular possible reference or control.

Risk: As used herein with respect to a disease, disorder, or condition, the term “risk” refers to the qualitative of quantitative probability (whether expressed as a percentage or otherwise) that a particular individual will develop the disease, disorder, or condition. In some embodiments, e.g., as set forth herein, risk is expressed as a percentage. In some embodiments, e.g., as set forth herein, a risk is a qualitative of quantitative probability that is equal to or greater than 0, 1, 2, 3, 4, 5, 6, 7, 8, 9, 10, 20, 30, 40, 50, 60, 70, 80, 90, or 100%. In some embodiments, e.g., as set forth herein, risk is expressed as a qualitative or quantitative level of risk relative to a reference risk or level or the risk of the same outcome attributed to a reference. In some embodiments, e.g., as set forth herein, relative risk is increased or decreased in comparison to the reference sample by a factor of 1.1, 1.2, 1.3, 1.4, 1.5, 1.6, 1.7, 1.8, 1.9, 2, 3, 4, 5, 6, 7, 8, 9, 10, or more.

Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample). Such a “processed sample” can include, for example cells, nucleic acids, or proteins extracted from a sample or obtained by subjecting a primary sample to techniques such as amplification or reverse transcription of nucleic acids, isolation and/or purification of certain components, etc.

In certain instances, e.g., as set forth herein, a processed sample can be a DNA sample that has been amplified (e.g., pre-amplified). Thus, in various instances, e.g., as set forth herein, an identified sample can refer to a primary form of the sample or to a processed form of the sample. In some instances, e.g., as set forth herein, a sample that is enzyme-digested DNA can refer to primary enzyme-digested DNA (the immediate product of enzyme digestion) or a further processed sample such as enzyme-digested DNA that has been subject to an amplification step (e.g., an intermediate amplification step, e.g., pre-amplification) and/or to a filtering step, purification step, or step that modifies the sample to facilitate a further step, e.g., in a process of determining methylation status (e.g., methylation status of a primary sample of DNA and/or of DNA as it existed in its original source context).

Screening: As used herein, the term “screening” refers to any method, technique, process, or undertaking intended to generate diagnostic information and/or prognostic information. Accordingly, those of skill in the art will appreciate that the term screening encompasses method, technique, process, or undertaking that determines whether an individual has, is likely to have or develop, or is at risk of having or developing a disease, disorder, or condition, e.g., colorectal cancer.

Specificity: As used herein, the “specificity” of a biomarker refers to the percentage of samples that are characterized by absence of the event or state of interest for which measurement of the biomarker accurately indicates absence of the event or state of interest (true negative rate). In various embodiments, e.g., as set forth herein, characterization of the negative samples is independent of the biomarker, and can be achieved by any relevant measure, e.g., any relevant measure known to those of skill in the art. Thus, specificity reflects the probability that the biomarker would detect the absence of the event or state of interest when measured in a sample not characterized that event or state of interest. In particular embodiments in which the event or state of interest is colorectal cancer, e.g., as set forth herein, specificity refers to the probability that a biomarker would detect the absence of colorectal cancer in a subject lacking colorectal cancer. Lack of colorectal cancer can be determined, e.g., by histology.

Sensitivity: As used herein, the “sensitivity” of a biomarker refers to the percentage of samples that are characterized by the presence of the event or state of interest for which measurement of the biomarker accurately indicates presence of the event or state of interest (true positive rate). In various embodiments, e.g., as set forth herein, characterization of the positive samples is independent of the biomarker, and can be achieved by any relevant measure, e.g., any relevant measure known to those of skill in the art. Thus, sensitivity reflects the probability that a biomarker would detect the presence of the event or state of interest when measured in a sample characterized by presence of that event or state of interest. In particular embodiments in which the event or state of interest is colorectal cancer, e.g., as set forth herein, sensitivity refers to the probability that a biomarker would detect the presence of colorectal cancer in a subject that has colorectal cancer. Presence of colorectal cancer can be determined, e.g., by histology.

Solid Tumor: As used herein, the term “solid tumor” refers to an abnormal mass of tissue including cancer cells. In various embodiments, e.g., as set forth herein, a solid tumor is or includes an abnormal mass of tissue that does not contain cysts or liquid areas. In some embodiments, e.g., as set forth herein, a solid tumor can be benign; in some embodiments, a solid tumor can be malignant. Examples of solid tumors include carcinomas, lymphomas, and sarcomas. In some embodiments, e.g., as set forth herein, solid tumors can be or include adrenal, bile duct, bladder, bone, brain, breast, cervix, colon, endometrium, esophagum, eye, gall bladder, gastrointestinal tract, kidney, larynx, liver, lung, nasal cavity, nasopharynx, oral cavity, ovary, penis, pituitary, prostate, retina, salivary gland, skin, small intestine, stomach, testis, thymus, thyroid, uterine, vaginal, and/or vulval tumors.

Stage of cancer: As used herein, the term “stage of cancer” refers to a qualitative or quantitative assessment of the level of advancement of a cancer. In some embodiments, e.g., as set forth herein, criteria used to determine the stage of a cancer can include, but are not limited to, one or more of where the cancer is located in a body, tumor size, whether the cancer has spread to lymph nodes, whether the cancer has spread to one or more different parts of the body, etc. In some embodiments, e.g., as set forth herein, cancer can be staged using the so-called TNM System, according to which T refers to the size and extent of the main tumor, usually called the primary tumor; N refers to the number of nearby lymph nodes that have cancer; and M refers to whether the cancer has metastasized. In some embodiments, e.g., as set forth herein, a cancer can be referred to as Stage 0 (abnormal cells are present but have not spread to nearby tissue, also called carcinoma in situ, or CIS; CIS is not cancer, but it can become cancer), Stage I-III (cancer is present; the higher the number, the larger the tumor and the more it has spread into nearby tissues), or Stage IV (the cancer has spread to distant parts of the body). In some embodiments, e.g., as set forth herein, a cancer can be assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); and unknown (there is not enough information to identify cancer stage).

Susceptible to: An individual who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status (e.g., a methylation status) associated with, development of the disease, disorder, or condition. In some embodiments, e.g., as set forth herein, a risk of developing a disease, disorder, and/or condition is a population-based risk (e.g., family members of individuals suffering from the disease, disorder, or condition).

Subject: As used herein, the term “subject” refers to an organism, typically a mammal (e.g., a human). In some embodiments, e.g., as set forth herein, a subject is suffering from a disease, disorder or condition. In some embodiments, e.g., as set forth herein, a subject is susceptible to a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”

Therapeutic agent: As used herein, the term “therapeutic agent” refers to any agent that elicits a desired pharmacological effect when administered to a subject. In some embodiments, e.g., as set forth herein, an agent is considered to be a therapeutic agent if it demonstrates a statistically significant effect across an appropriate population. In some embodiments, e.g., as set forth herein, the appropriate population can be a population of model organisms or a human population. In some embodiments, e.g., as set forth herein, an appropriate population can be defined by various criteria, such as a certain age group, gender, genetic background, preexisting clinical conditions, etc. In some embodiments, e.g., as set forth herein, a therapeutic agent is a substance that can be used for treatment of a disease, disorder, or condition. In some embodiments, e.g., as set forth herein, a therapeutic agent is an agent that has been or is required to be approved by a government agency before it can be marketed for administration to humans. In some embodiments, e.g., as set forth herein, a therapeutic agent is an agent for which a medical prescription is required for administration to humans.

Therapeutically effective amount: As used herein, the term “therapeutically effective amount” refers to an amount that produces a desired effect for which it is administered. In some embodiments, e.g., as set forth herein, the term refers to an amount that is sufficient, when administered to a population suffering from or susceptible to a disease, disorder, or condition, in accordance with a therapeutic dosing regimen, to treat the disease, disorder, or condition. Those of ordinary skill in the art will appreciate that the term therapeutically effective amount does not in fact require successful treatment be achieved in a particular individual. Rather, a therapeutically effective amount can be an amount that provides a particular desired pharmacological response in a significant number of subjects when administered to individuals in need of such treatment. In some embodiments, e.g., as set forth herein, reference to a therapeutically effective amount can be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent can be formulated and/or administered in a single dose. In some embodiments, e.g., as set forth herein, a therapeutically effective agent can be formulated and/or administered in a plurality of doses, for example, as part of a multi-dose dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, e.g., as set forth herein, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, e.g., as set forth herein, treatment can be of a subject who has been diagnosed as suffering from the relevant disease, disorder, and/or condition. In some embodiments, e.g., as set forth herein, treatment can be of a subject known to have one or more susceptibility factors that are statistically correlated with increased risk of development of the relevant disease, disorder, or condition. In various examples, treatment is of a cancer.

Upstream: As used herein, the term “upstream” means a first DNA region is closer, relative to a second DNA region, to the N-terminus of a nucleic acid that includes the first DNA region and the second DNA region.

Unit dose: As used herein, the term “unit dose” refers to an amount administered as a single dose and/or in a physically discrete unit of a pharmaceutical composition. In many embodiments, e.g., as set forth herein, a unit dose contains a predetermined quantity of an active agent. In some embodiments, e.g., as set forth herein, a unit dose contains an entire single dose of the agent. In some embodiments, e.g., as set forth herein, more than one unit dose is administered to achieve a total single dose. In some embodiments, e.g., as set forth herein, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic agent(s). For example, acceptable carriers (e.g., pharmaceutically acceptable carriers), diluents, stabilizers, buffers, preservatives, etc., can be included. It will be appreciated by those skilled in the art, in many embodiments, e.g., as set forth herein, a total appropriate daily dosage of a particular therapeutic agent can comprise a portion, or a plurality, of unit doses, and can be decided, for example, by a medical practitioner within the scope of sound medical judgment. In some embodiments, e.g., as set forth herein, the specific effective dose level for any particular subject or organism can depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unmethylated: As used herein, the terms “unmethylated” and “non-methylated” are used interchangeable and mean that an identified DNA region includes no methylated nucleotides.

Variant: As used herein, the term “variant” refers to an entity that shows significant structural identity with a reference entity but differs structurally from the reference entity in the presence, absence, or level of one or more chemical moieties as compared with the reference entity. In some embodiments, e.g., as set forth herein, a variant also differs functionally from its reference entity. In general, whether a particular entity is properly considered to be a “variant” of a reference entity is based on its degree of structural identity with the reference entity. A variant can be a molecule comparable, but not identical to, a reference. For example, a variant nucleic acid can differ from a reference nucleic acid at one or more differences in nucleotide sequence. In some embodiments, e.g., as set forth herein, a variant nucleic acid shows an overall sequence identity with a reference nucleic acid that is at least 85%, 86%, 87%, 88%, 89%, 90%, 91%, 92%, 93%, 94%, 95%, 96%, 97%, or 99%. In many embodiments, e.g., as set forth herein, a nucleic acid of interest is considered to be a “variant” of a reference nucleic acid if the nucleic acid of interest has a sequence that is identical to that of the reference but for a small number of sequence alterations at particular positions. In some embodiments, e.g., as set forth herein, a variant has 10, 9, 8, 7, 6, 5, 4, 3, 2, or 1 substituted residues as compared with a reference. In some embodiments, e.g., as set forth herein, a variant has not more than 5, 4, 3, 2, or 1 residue additions, substitutions, or deletions as compared with the reference. In various embodiments, e.g., as set forth herein, the number of additions, substitutions, or deletions is fewer than about 25, about 20, about 19, about 18, about 17, about 16, about 15, about 14, about 13, about 10, about 9, about 8, about 7, about 6, and commonly are fewer than about 5, about 4, about 3, or about 2 residues.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a graph showing boxplots of the values in each condition group based on a 4-methylation region combination. The horizontal line traversing across the graph indicates 90% specificity line.

FIG. 2 is a graph showing the sensitivity of detection of identified disease groups per progression stage and specificity for a control condition.

FIG. 3 is a graph showing a boxplot showing sample values in each condition group based on a second 4-methylation region combination. The horizontal line traversing across the graph indicates 90% specificity line.

FIGS. 4A-F are graphs showing boxplots representing the percent methylation of a DMR region in samples corresponding to identified conditions.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

### Screening for Colorectal Neoplasms

There is a need for improved methods of detecting (e.g., screening for) colorectal neoplasms including, but not limited to, advanced adenoma, polyposis, and/or colorectal cancer. This includes a need for screening for early-stage colorectal cancer. Despite recommendations for screening of individuals, e.g., over age 45, colorectal cancer screening programs are often ineffective or unsatisfactory. Improved screens for colorectal neoplasms improves diagnosis and reduces colorectal cancer mortality.

DNA methylation (e.g., hypermethylation or hypomethylation) can activate or inactivate genes, including genes that impact development of neoplasms, including cancers. Thus, for example, hypermethylation can inactivate one or more genes that typically act to suppress cancer, causing or contributing to development of cancer in a sample or subject.

The present disclosure includes the discovery that determination of the methylation status of one or more methylation loci provided herein, and/or the methylation status of one or more DMRs provided herein, provides for detection of (e.g., screening for) colorectal neoplasms including, but not limited to, advanced adenoma, polyposis, and/or colorectal cancer (e.g., early stage colorectal cancer). In certain embodiments, screening can classify a subject as having a colorectal neoplasms a high degree of sensitivity and/or specificity that a subject has or does not have one or more conditions. The present disclosure provides compositions and methods including or relating to colorectal neoplasm biomarkers including advanced adenoma, polyposis and/or colorectal cancer methylation biomarkers that, individually or in various panels comprising two or more methylation biomarkers, provide for screening of advanced adenoma, polyposis and/or colorectal cancer (e.g., early stage colorectal cancer).

In various embodiments, a methylation biomarker of the present disclosure used for detection of colorectal neoplasms including advanced adenoma, polyposis and/or colorectal cancer is selected from a methylation locus that is or includes at least a portion of a DMR listed in Table 1. Table 1 lists the region of DNA on which the DMR is found, which includes the chromosome number (chr), the start and end positions of the DMR on the chromosome, and genes (if any) that are known to be associated with the region. If no genes are currently known to be associated with the region, the term “NA” is listed in the Associated Genes column. Each DMR is also provided with a unique identifying number (uid) that can be used to unambiguously identify the DMR. Additionally, the size (e.g., length) of the DMR region (“size of the region”) is also listed.

TABLE 1
List of DMRs found to have significantly altered methylation pattern(s) in the blood and/or
tissue of colorectal cancer and/or advanced adenoma patients compared to controls.

| | | | | | | Size |
|---|---|---|---|---|---|---|
| | | | Associated | SEQ | | of the |
| Chr | Start | End | Genes | ID NO: | uid | region |
| 1 | 34165443 | 34165675 | CSMD2, | 1 | 1_34165443_34165675 | 233 |
| | | | C1orf94 | | | |
| 1 | 219928356 | 219928386 | SLC30A10 | 2 | 1_219928356_219928386 | 31 |
| 1 | 161539915 | 161540181 | FCGR2A | 3 | 1_161539915_161540181 | 267 |
| 1 | 150294178 | 150294265 | MRPS21 | 4 | 1_150294178_150294265 | 88 |
| 2 | 47569627 | 47569858 | MSH2, | 5 | 2_47569627_47569858 | 232 |
| | | | KCNK12 | | | |
| 3 | 157437029 | 157437296 | VEPH1, | 6 | 3_157437029 157437296 | 268 |
| | | | PTX3 | | | |
| 3 | 173397284 | 173397387 | NLGN1 | 7 | 3_173397284_173397387 | 104 |
| 3 | 43998153 | 43998214 | NA | 8 | 3_43998153_43998214 | 62 |
| 3 | 10993689 | 10993900 | SLC6A1 | 9 | 3_10993689_10993900 | 212 |
| 3 | 181712580 | 181712614 | SOX2, | 10 | 3_181712580_181712614 | 35 |
| | | | SOX2-OT | | | |
| 3 | 180679674 | 180679706 | CCDC39, | 11 | 3_180679674_180679706 | 33 |
| | | | LOC101928882 | | | |
| 7 | 65252405 | 65252749 | NA | 12 | 7_65252405_65252749 | 345 |
| 7 | 64314242 | 64314346 | ZNF736 | 13 | 7_64314242_64314346 | 105 |
| 8 | 53877760 | 53878892 | RGS20 | 14 | 8_53877760_53878892 | 1133 |
| 8 | 66177597 | 66177765 | CRH | 15 | 8_66177597_66177765 | 169 |
| 8 | 108787618 | 108787717 | TMEM74 | 16 | 8_108787618_108787717 | 100 |
| 8 | 53881543 | 53881774 | RGS20 | 17 | 8_53881543_53881774 | 232 |
| 4 | 183905237 | 183905728 | STOX2 | 18 | 4_183905237_183905728 | 492 |
| 4 | 183904880 | 183905140 | STOX2 | 19 | 4_183904880_183905140 | 261 |
| 4 | 13545274 | 13545403 | NKX3-2 | 20 | 4_13545274_13545403 | 130 |
| 4 | 127623100 | 127623235 | INTU | 21 | 4_127623100_127623235 | 136 |
| 4 | 30718010 | 30718076 | PCDH7 | 22 | 4_30718010_30718076 | 67 |
| 4 | 82562331 | 82562477 | TMEM150C | 23 | 4_82562331_82562477 | 147 |
| 5 | 88660609 | 88660834 | LINC00461 | 24 | 5_88660609_88660834 | 226 |
| 5 | 83474370 | 83474485 | VCAN | 25 | 5_83474370_83474485 | 116 |
| 5 | 79512985 | 79513164 | HOMER1 | 26 | 5_79512985_79513164 | 180 |
| 5 | 51387638 | 51388080 | LOC642366, | 27 | 5_51387638_51388080 | 443 |
| | | | ISL1 | | | |
| 6 | 129767354 | 129767459 | NA | 28 | 6_129767354_129767459 | 106 |
| 6 | 27288050 | 27288544 | NA | 29 | 6_27288050_27288544 | 495 |
| 6 | 165309346 | 165309582 | C6orf118 | 30 | 6_165309346_165309582 | 237 |
| 6 | 6320205 | 6320663 | F13A1 | 31 | 6_6320205_6320663 | 459 |
| 6 | 68635305 | 68635560 | ADGRB3, | 32 | 6_68635305_68635560 | 256 |
| | | | LOC101928307 | | | |
| 6 | 27670532 | 27670614 | NA | 33 | 6_27670532_27670614 | 83 |
| 6 | 1524106 | 1524152 | NA | 34 | 6_1524106_1524152 | 47 |
| 11 | 79438095 | 79438308 | TENM4 | 35 | 11_79438095_79438308 | 214 |
| 11 | 94740046 | 94740862 | LOC105369438, | 36 | 11_94740046_94740862 | 817 |
| | | | AMOTL1 | | | |
| 13 | 93228702 | 93229444 | GPC6 | 37 | 13_93228702_93229444 | 743 |
| 13 | 94712995 | 94713032 | SOX21, | 38 | 13_94712995_94713032 | 38 |
| | | | SOX21-AS1 | | | |
| 13 | 67231493 | 67231965 | PCDH9 | 39 | 13_67231493_67231965 | 473 |
| 13 | 61414743 | 61414845 | PCDH20 | 40 | 13_61414743_61414845 | 103 |
| 9 | 132579614 | 132579683 | BARHL1 | 41 | 9_132579614_132579683 | 70 |
| 10 | 22475853 | 22476043 | NA | 42 | 10_22475853_22476043 | 191 |
| 10 | 117165171 | 117165431 | MIR3663HG | 43 | 10_117165171_117165431 | 261 |
| 10 | 16521408 | 16521447 | C1QL3 | 44 | 10_16521408_16521447 | 40 |
| 12 | 78865146 | 78865306 | SYT1 | 45 | 12_78865146_78865306 | 161 |
| 12 | 15221303 | 15221461 | RERG | 46 | 12_15221303_15221461 | 159 |
| 12 | 24563707 | 24564068 | SOX5 | 47 | 12_24563707_24564068 | 362 |
| 12 | 53974529 | 53974571 | HOXC11, | 48 | 12_53974529_53974571 | 43 |
| | | | HOTAIR | | | |
| 14 | 59870236 | 59870262 | RTN1 | 49 | 14_59870236_59870262 | 27 |
| 18 | 27184277 | 27184863 | AQP4-AS1, | 50 | 18_27184277_27184863 | 587 |
| | | | CHST9 | | | |
| 20 | 9516555 | 9516632 | LAMP5-AS1, | 51 | 20_9516555_9516632 | 78 |
| | | | LAMP5 | | | |
| 21 | 31344104 | 31344160 | TIAM1 | 52 | 21_31344104_31344160 | 57 |
| 19 | 20052466 | 20053193 | NA | 53 | 19_20052466_20053193 | 728 |
| X | 625391 | 625465 | SHOX | 54 | X_625391_625465 | 75 |

For the avoidance of any doubt, any methylation biomarker provided herein can be, or be included in, among other things, a colorectal neoplasm marker. Additionally, any methylation biomarker herein can be, or be included in, an advanced adenoma, polyposis, and/or colorectal cancer (e.g., early stage colorectal cancer) methylation biomarker.

In some embodiments, said methylation biomarker can be or include a single methylation locus. In some embodiments, a methylation biomarker can be or include two or more methylation loci. In some embodiments, a methylation biomarker can be or include a single differentially methylated region (DMR) (e.g., (i) a DMR selected from those listed in Table 1, (ii) a DMR that encompasses a DMR selected from those listed in Table 1, (iii) a DMR that overlaps with one or more DMRs selected from those listed in Table 1, or (iv) a DMR that is a portion of a DMR selected from those listed in Table 1). In some embodiments, a methylation locus can be or include two or more DMRs (e.g., two, three, four, or more DMRs selected from those listed in Table 1, or two, three, four, or more DMRs, each of which overlap with and/or encompass a DMR selected from those listed in Table 1). In some embodiments, a methylation biomarker can be or include a single methylation site. In other embodiments, a methylation biomarker can be or include two or more methylation sites. In some embodiments, a methylation locus can include two or more DMRs and further include DNA regions adjacent to one or more of the included DMRs.

In some instances, a methylation locus is or includes a gene, such as a gene provided in Table 1. In some instances a methylation locus is or includes a portion of a gene, e.g., a portion of a gene provided in Table 1. In some instances, a methylation locus includes but is not limited to identified nucleic acid boundaries of a gene.

In some instances, a methylation locus is or includes a coding region of a gene, such as a coding region of a gene provided in Table 1. In some instances a methylation locus is or includes a portion of the coding region of gene, e.g., a portion of the coding region a gene provided in Table 1. In some instances, a methylation locus includes but is not limited to identified nucleic acid boundaries of a coding region of gene.

In some instances, a methylation locus is or includes a promoter and/or other regulatory region of a gene, such as a promoter and/or other regulatory region of a gene provided in Table 1. In some instances a methylation locus is or includes a portion of the promoter and/or regulatory region of a gene, e.g., a portion of promoter and/or regulatory region a gene provided in Table 1. In some instances, a methylation locus includes but is not limited to identified nucleic acid boundaries of a promoter and/or other regulatory region of gene. In some embodiments a methylation locus is or includes a high CpG density promoter, or a portion thereof.

In some embodiments, a methylation locus is or includes non-coding sequence. In some embodiments, a methylation locus is or includes one or more exons, and/or one or more introns.

In some embodiments, a methylation locus includes a DNA region extending a predetermined number of nucleotides upstream of a coding sequence, and/or a DNA region extending a predetermined number of nucleotides downstream of a coding sequence. In various instances, a predetermined number of nucleotides upstream and/or downstream and be or include, e.g., 500 bp, 1 kb, 2 kb, 3 kb, 4 kb, 5 kb, 10 kb, 20 kb, 30 kb, 40 kb, 50 kb, 75 kb, or 100 kb. Those of skill in the art will appreciate that methylation biomarkers capable of impacting expression of a coding sequence may typically be within any of these distances of the coding sequence, upstream and/or downstream.

Those of skill in the art will appreciate that a methylation locus identified as a methylation biomarker need not necessarily be assayed in a single experiment, reaction, or amplicon. A single methylation locus identified as a colorectal cancer methylation biomarker can be assayed, e.g., in a method including separate amplification (or providing oligonucleotide primers and conditions sufficient for amplification of) of one or more distinct or overlapping DNA regions within a methylation locus, e.g., one or more distinct or overlapping DMRs. Those of skill in the art will further appreciate that a methylation locus identified as a methylation biomarker need not be analyzed for methylation status of each nucleotide, nor each CpG, present within the methylation locus. Rather, a methylation locus that is a methylation biomarker may be analyzed, e.g., by analysis of a single DNA region within the methylation locus, e.g., by analysis of a single DMR within the methylation locus.

DMRs of the present disclosure can be a methylation locus or include a portion of a methylation locus. In some instances, a DMR is a DNA region with a methylation locus that is, e.g., 1 to 5,000 bp in length. In various embodiments, a DMR is a DNA region with a methylation locus that is equal to or less than 5000 bp, 4,000 bp, 3,000 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp in length. In some embodiments, a DMR is 1, 2, 3, 4, 5, 6, 7, 8 or 9 bp in length.

Methylation biomarkers, including without limitation methylation loci and DMRs provided herein, can include at least one methylation site that is a colorectal neoplasm biomarker.

For clarity, those of skill in the art will appreciate that term methylation biomarker is used broadly, such that a methylation locus can be a methylation biomarker that includes one or more DMRs, each of which DMRs is also itself a methylation biomarker, and each of which DMRs can include one or more methylation sites, each of which methylation sites is also itself a methylation biomarker. Moreover, a methylation biomarker can include two or more methylation loci. Accordingly, status as a methylation biomarker does not turn on the contiguousness of nucleic acids included in a biomarker, but rather on the existence of a change in methylation status for included DNA region(s) between a first state and a second state, such as between colorectal cancer and controls.

As provided herein, a methylation locus can be any of one or more methylation loci each of which methylation loci is, includes, or is a portion of a gene (or specific DMR) identified in Table 1. In some embodiments, a colorectal neoplasm methylation biomarker (e.g., an advanced adenoma, a polyposis, and/or a colorectal cancer (e.g., early stage colorectal cancer) methylation biomarker) includes a single methylation locus that is, includes, or is a portion of a gene identified in Table 1.

In some embodiments, a methylation biomarker includes two or more methylation loci, each of which is, includes, or is a portion of a gene identified in Table 1. In some embodiments, a colorectal neoplasm methylation biomarker (e.g., an advanced adenoma, polyposis, and/or colorectal cancer methylation biomarker) includes a plurality of methylation loci, each of which is, includes, or is a portion of a gene identified in Table 1.

In various embodiments, a methylation biomarker can be or include one or more individual nucleotides (e.g., a single individual cysteine residue in the context of CpG) or a plurality of individual cysteine residues (e.g., of a plurality of CpGs) present within one or more methylation loci (e.g, one or more DMRs) provided herein. Thus, in certain embodiments a methylation biomarker is or includes methylation status of a plurality of individual methylation sites.

In various embodiments, a methylation biomarker is, includes, or is characterized by change in methylation status that is a change in the methylation of one or more methylation sites within one or more methylation loci (e.g., one or more DMRs). In various embodiments, a methylation biomarker is or includes a change in methylation status that is a change in the number of methylated sites within one or more methylation loci (e.g., one or more DMRs). In various embodiments, a methylation biomarker is or includes a change in methylation status that is a change in the frequency of methylation sites within one or more methylation loci (e.g., one or more DMRs). In various embodiments, a methylation biomarker is or includes a change in methylation status that is a change in the pattern of methylation sites within one or more methylation loci (e.g., one or more DMRs).

In various embodiments, methylation status of one or more methylation loci (e.g., one or more DMRs) is expressed as a fraction or percentage of the one or more methylation loci (e.g., the one or more DMRs) present in a sample that are methylated, e.g., as a fraction of the number of individual DNA strands of DNA in a sample that are methylated at one or more particular methylation loci (e.g., one or more particular DMRs). Those of skill in the art will appreciate that, in some instances, the fraction or percentage of methylation can be calculated from the ratio of methylated DMRs to unmethylated DMRs for one or more analyzed DMRs, e.g., within a sample.

In various embodiments, methylation status of one or more methylation loci (e.g., one or more DMRs) is compared to a reference methylation status value and/or to methylation status of the one or more methylation loci (e.g., one or more DMRs) in a reference sample or a group of reference samples. For example, in certain embodiments, the group of reference samples is a plurality of samples obtained from individuals where said samples are known to represent a particular state (e.g., a “normal” non-cancer state, or a cancer state). In certain instances, a reference is a non-contemporaneous sample from the same source, e.g., a prior sample from the same source, e.g., from the same subject. In certain instances, a reference for the methylation status of one or more methylation loci (e.g., one or more DMRs) is the methylation status of the one or more methylation loci (e.g., one or more DMRs) in a sample (e.g., a sample from a subject), or a plurality of samples, known to represent a particular state (e.g., a cancer state or a non-cancer state). Thus, a reference can be or include one or more predetermined thresholds, which thresholds can be quantitative (e.g., a methylation value) or qualitative. Those of skill in the art will appreciate that a reference measurement is typically produced by measurement using a methodology identical to, similar to, or comparable to that by which the non-reference measurement was taken.

### Advanced Adenomas

In certain embodiments, methods and compositions presented herein are useful for screening for advanced adenomas. Advanced adenomas include, without limitation: neoplastic adenomatous growth in colon and/or in rectum, adenomas located in the proximal part of the colon, adenomas located in the distal part of the colon and/or rectum, adenomas of low grade dysplasia, adenomas of high grade dysplasia, neoplastic growth(s) of colorectum tissue that shows signs of high grade dysplasia of any size, neoplastic growth(s) of colorectum tissue having a size greater than or equal to 10 mm of any histology and/or dysplasia grade, neoplastic growth(s) of colorectum tissue with villious histological type of any type of dysplasia and any size, and colorectum tissue having a serrated histological type with any dysplasia grade and/or size.

### Cancers

In certain embodiments, methods and compositions of the present disclosure are useful for screening for colorectal cancer. Colorectal cancers include, without limitation, colon cancer, rectal cancer, and combinations thereof. Colorectal cancers include metastatic colorectal cancers and non-metastatic colorectal cancers. Colorectal cancers include cancer located in the proximal part of the colon cancer and cancer located in the distal part of the colon.

Colorectal cancers include colorectal cancers at any of the various possible stages known in the art, including, e.g., Stage I, Stage II, Stage III, and Stage IV colorectal cancers (e.g., stages 0, I, IIA, IIB, IIC, IIIA, IIIB, IIIC, IVA, IVB, and IVC). Colorectal cancers include all stages of the Tumor/Node/Metastasis (TNM) staging system. With respect to colorectal cancer, T can refer to whether the tumor grown into the wall of the colon or rectum, and if so by how many layers; N can refer to whether the tumor has spread to lymph nodes, and if so how many lymph nodes and where they are located; and M can refer to whether the cancer has spread to other parts of the body, and if so which parts and to what extent. Particular stages of T, N, and M are known in the art. T stages can include TX, T0, Tis, T1, T2, T3, T4a, and T4b; N stages can include NX, N0, N1a, N1b, N1c, N2a, and N2b; M stages can include M0, M1a, and M1b. Moreover, grades of colorectal cancer can include GX, G1, G2, G3, and G4. Various means of staging cancer, and colorectal cancer in particular, are well known in the art summarized, e.g., on the world wide web at cancer.net/cancer-types/colorectal-cancer/stages.

In certain instances, the present disclosure includes screening of early stage colorectal cancer. Early stage colorectal cancers can include, e.g., colorectal cancers localized within a subject, e.g., in that they have not yet spread to lymph nodes of the subject, e.g., lymph nodes near to the cancer (stage N0), and have not spread to distant sites (stage M0). Early stage cancers include colorectal cancers corresponding to, e.g., Stages 0 to II C.

Thus, colorectal cancers of the present disclosure include, among other things, pre-malignant colorectal cancer and malignant colorectal cancer. Methods and compositions of the present disclosure are useful for screening of colorectal cancer in all of its forms and stages, including without limitation those named herein or otherwise known in the art, as well as all subsets thereof. Accordingly, the person of skill in art will appreciate that all references to colorectal cancer provided here include, without limitation, colorectal cancer in all of its forms and stages, including without limitation those named herein or otherwise known in the art, as well as all subsets thereof.

### Polyposis

In certain embodiments, methods and compositions of the present disclosure are useful for screening for polyposis (e.g., polyposis syndromes).

Polyposis includes hereditary conditions that result in an individual being more prone to development of multiple polyps (e.g., more than 10 polyps). These polyps are generally found in the individual's colon and/or rectum. A number of hereditary conditions can be classified as polyposis syndromes including, without limitation: familial adenomatous polyposis (FAP), hereditary nonpolyposis colorectal cancer (HNPCC)/Lynch syndrome, Gardner syndrome, Turcot syndrome, MUTYH polyposis, Peutz-Jeghers syndrome, Cowden disease, familial juvenile polyposis, serrated polyposis syndrome (SPS), and hyperplastic polyposis.

Serrated polyposis syndrome (SPS), which is a polyposis syndrome, can be identified by an individual having: 5 or more serrated polyps proximal to the sigmoid colon with two or more at least 10 mm in size, a serrated polyp proximal to the sigmoid colon in the context of a family history of serrated polyposis, and/or 20 or more serrated polyps throughout the colon and rectum.

### Subjects and Samples

A sample analyzed using methods and compositions provided herein can be any biological sample and/or any sample including nucleic acids. In various particular embodiments, a sample analyzed using methods and compositions provided herein can be a sample from a mammal. In various particular embodiments, a sample analyzed using methods and compositions provided herein can be a sample from a human subject. In various particular embodiments, a sample analyzed using methods and compositions provided herein can be a sample form a mouse, rat, pig, horse, chicken, or cow.

In various instances, a human subject is a subject diagnosed or seeking diagnosis as having, diagnosed as or seeking diagnosis as at risk of having, and/or diagnosed as or seeking diagnosis as at immediate risk of having, a colorectal neoplasm (e.g., colorectal cancer). In various instances, a human subject is a subjected identified as a subject in need of screening for a colorectal neoplasm (e.g., colorectal cancer). In certain instances, a human subject is a subject identified as in need of colorectal cancer screening by a medical practitioner. In various instances, a human subject is identified as in need of colorectal cancer screening due to age, e.g., due to an age equal to or greater than 45 years, e.g., an age equal to or greater than 45, 50, 55, 60, 65, 70, 75, 80, 85, or 90 years, though in some instances a subject 18 years old or older may be identified as at risk and/or in need of screening for a colorectal neoplasm (e.g., colorectal cancer). In various instances, a human subject is identified as being high risk and/or in need of screening for a colorectal neoplasm (e.g., colorectal cancer) based on, without limitation, familial history, prior diagnoses, and/or an evaluation by a medical practitioner. In various instances, a human subject is a subject not diagnosed as having, not at risk of having, not at immediate risk of having, not diagnosed as having, and/or not seeking diagnosis for a cancer such as a colorectal cancer, or any combination thereof.

A sample from a subject, e.g., a human or other mammalian subject, can be a sample of, e.g., blood, blood component (e.g., plasma, buffy coat), cfDNA (cell free DNA), ctDNA (circulating tumor DNA), stool, or advanced adenoma and/or colorectal tissue. In some particular embodiments, a sample is an excretion or bodily fluid of a subject (e.g., stool, blood, plasma, lymph, or urine of a subject) or a tissue sample of a colorectal neoplasm, such as a colonic polyp, an advanced adenoma, and/or colorectal cancer. A sample from a subject can be a cell or tissue sample, e.g., a cell or tissue sample that is of a cancer or includes cancer cells, e.g., of a tumor or of a metastatic tissue. In various embodiments, a sample from a subject, e.g., a human or other mammalian subject, can be obtained by biopsy (e.g., colonoscopy resection, fine needle aspiration or tissue biopsy) or surgery.

In various particular embodiments, a sample is a sample of cell-free DNA (cfDNA). cfDNA is typically found in biological fluids (e.g., plasma, serum, or urine) in short, double-stranded fragments. The concentration of cfDNA is typically low, but can significantly increase under particular conditions, including without limitation pregnancy, autoimmune disorder, myocardial infraction, and cancer. Circulating tumor DNA (ctDNA) is the component of circulating DNA specifically derived from cancer cells. ctDNA can be present in human fluids. For example in some instances, ctDNA can be found bound to and/or associated with leukocytes and erythrocytes. In some instances, ctDNA can be found not bound to and/or associated with leukocytes and erythrocytes. Various tests for detection of tumor-derived cfDNA are based on detection of genetic or epigenetic modifications that are characteristic of cancer (e.g., of a relevant cancer). Genetic or epigenetic modifications characteristic of cancer can include, without limitation, oncogenic or cancer-associated mutations in tumor-suppressor genes, activated oncogenes, hypermethylation, and/or chromosomal disorders. Detection of genetic or epigenetic modifications characteristic of cancer or pre-cancer can confirm that detected cfDNA is ctDNA.

cfDNA and ctDNA provide a real-time or nearly real-time metric of the methylation status of a source tissue. cfDNA and ctDNA have a half-life in blood of about 2 hours, such that a sample taken at a given time provides a relatively timely reflection of the status of a source tissue.

Various methods of isolating nucleic acids from a sample (e.g., of isolating cfDNA from blood or plasma) are known in the art. Nucleic acids can be isolated, e.g., without limitation, standard DNA purification techniques, by direct gene capture (e.g., by clarification of a sample to remove assay-inhibiting agents and capturing a target nucleic acid, if present, from the clarified sample with a capture agent to produce a capture complex, and isolating the capture complex to recover the target nucleic acid).

### Methods of Measuring Methylation Status

Methylation status can be measured by a variety of methods known in the art and/or by methods provided herein. Those of skill in the art will appreciate that a method for measuring methylation status can generally be applied to samples from any source and of any kind, and will further be aware of processing steps available to modify a sample into a form suitable for measurement by a given methodology. Methods of measuring methylation status include, without limitation, methods including whole genome bisulfite sequencing, targeted bisulfite sequencing, targeted enzymatic methylation sequencing, methylation-status-specific polymerase chain reaction (PCR), methods including mass spectrometry, methylation arrays, methods including methylation-specific nucleases, methods including mass-based separation, methods including target-specific capture, and methods including methylation-specific oligonucleotide primers. Certain particular assays for methylation utilize a bisulfite reagent (e.g., hydrogen sulfite ions) or enzymatic conversion reagents (e.g., Tet methylcytosine dioxygenase 2).

Bisulfite reagents can include, among other things, bisulfite, disulfite, hydrogen sulfite, or combinations thereof, which reagents can be useful in distinguishing methylated and unmethylated nucleic acids. Bisulfite interacts differently with cytosine and 5-methylcytosine. In typical bisulfite-based methods, contacting of DNA with bisulfite deaminates unmethylated cytosine to uracil, while methylated cytosine remains unaffected; methylated cytosines, but not unmethylated cytosines, are selectively retained. Thus, in a bisulfite processed sample, uracil residues stand in place of, and thus provide an identifying signal for, unmethylated cytosine residues, while remaining (methylated) cytosine residues thus provide an identifying signal for methylated cytosine residues. Bisulfite processed samples can be analyzed, e.g., by next generation sequencing (NGS).

Enzymatic conversion reagents can include Tet methylcytosine dioxygenase 2 (TET2). TET2 oxidizes 5-methylcytosine and thus protects it from the consecutive deamination by APOBEC. APOBEC deaminates unmethylated cytosine to uracil, while oxidized 5-methylcytosine remains unaffected. Thus, in a TET2 processed sample, uracil residues stand in place of, and thus provide an identifying signal for, unmethylated cytosine residues, while remaining (methylated) cytosine residues thus provide an identifying signal for methylated cytosine residues. TET2 processed samples can be analyzed, e.g., by next generation sequencing (NGS).

Methods of measuring methylation status can include, without limitation, massively parallel sequencing (e.g., next-generation sequencing) to determine methylation state, e.g., sequencing by-synthesis, real-time (e.g., single-molecule) sequencing, bead emulsion sequencing, nanopore sequencing, or other sequencing techniques known in the art. In some embodiments, a method of measuring methylation status can include whole-genome sequencing, e.g., measuring whole genome methylation status from bisulfite or enzymatically treated material with base-pair resolution.

In some embodiments, a method of measuring methylation status includes reduced representation bisulfite sequencing e.g., utilizing use of restriction enzymes to measure methylation status of high CpG content regions from bisulfite or enzymatically treated material with base-pair resolution.

In some embodiments, a method of measuring methylation status can include targeted sequencing e.g., measuring methylation status of pre-selected genomic location from bisulfite or enzymatically treated material with base-pair resolution.

In some embodiments, the pre-selection (capture) of regions of interest can be done by complementary in vitro synthesized oligonucleotide sequences (either baits, primers or probes).

In some embodiments, a method for measuring methylation status can include Illumina Methylation Assays e.g., measuring over 850,000 methylation sites quantitatively across a genome at single-nucleotide resolution.

Various methylation assay procedures can be used in conjunction with bisulfite treatment to determine methylation status of a target sequence such as a DMR. Such assays can include, among others, Methylation-Specific Restriction Enzyme qPCR, sequencing of bisulfite-treated nucleic acid, PCR (e.g., with sequence-specific amplification), Methylation Specific Nuclease-assisted Minor-allele Enrichment PCR, and Methylation-Sensitive High Resolution Melting. In some embodiments, DMRs are amplified from a bisulfite-treated DNA sample and a DNA sequencing library is prepared for sequencing according to, e.g., an Illumina protocol or transpose-based Nextera XT protocol. In certain embodiments, high-throughput and/or next-generation sequencing techniques are used to achieve base-pair level resolution of DNA sequence, permitting analysis of methylation status.

Another method, that can be used for methylation detection includes PCR amplification with methylation-specific oligonucleotide primers (MSP methods), e.g., as applied to bisulfite-treated sample (see, e.g., Herman 1992 Proc. Natl. Acad. Sci. USA 93: 9821-9826, which is herein incorporated by reference with respect to methods of determining methylation status). Use of methylation-status-specific oligonucleotide primers for amplification of bisulfite-treated DNA allows differentiation between methylated and unmethylated nucleic acids. Oligonucleotide primer pairs for use in MSP methods include at least one oligonucleotide primer capable of hybridizing with sequence that includes a methylation site, e.g., a CpG. An oligonucleotide primer that includes a T residue at a position complementary to a cytosine residue will selectively hybridize to templates in which the cytosine was unmethylated prior to bisulfite treatment, while an oligonucleotide primer that includes a G residue at a position complementary to a cytosine residue will selectively hybridize to templates in which the cytosine was methylated cytosine prior to bisulfite treatment. MSP results can be obtained with or without sequencing amplicons, e.g., using gel electrophoresis. MSP (methylation-specific PCR) allows for highly sensitive detection (detection level of 0.1% of the alleles, with full specificity) of locus-specific DNA methylation, using PCR amplification of bisulfite-converted DNA.

Another method that can be used to determine methylation status after bisulfite treatment of a sample is Methylation-Sensitive High Resolution Melting (MS-HRM) PCR (see, e.g., Hussmann 2018 Methods Mol Biol. 1708:551-571, which is herein incorporated by reference with respect to methods of determining methylation status). MS-HRM is an in-tube, PCR-based method to detect methylation levels at specific loci of interest based on hybridization melting. Bisulfite treatment of the DNA prior to performing MS-HRM ensures a different base composition between methylated and unmethylated DNA, which is used to separate the resulting amplicons by high resolution melting. A unique primer design facilitates a high sensitivity of the assays enabling detection of down to 0.1-1% methylated alleles in an unmethylated background. Oligonucleotide primers for MS-HRM assays are designed to be complementary to the methylated allele, and a specific annealing temperature enables these primers to anneal both to the methylated and the unmethylated alleles thereby increasing the sensitivity of the assays.

Another method that can be used to determine methylation status after bisulfite treatment of a sample is Quantitative Multiplex Methylation-Specific PCR (QM-MSP). QM-MSP uses methylation specific primers for sensitive quantification of DNA methylation (see, e.g., Fackler 2018 Methods Mol Biol. 1708:473-496, which is herein incorporated by reference with respect to methods of determining methylation status). QM-MSP is a two-step PCR approach, where in the first step, one pair of gene-specific primers (forward and reverse) amplifies the methylated and unmethylated copies of the same gene simultaneously and in multiplex, in one PCR reaction. This methylation-independent amplification step produces amplicons of up to 10⁹ copies per μL after 36 cycles of PCR. In the second step, the amplicons of the first reaction are quantified with a standard curve using real-time PCR and two independent fluorophores to detect methylated/unmethylated DNA of each gene in the same well (e.g., 6FAM and VIC). One methylated copy is detectable in 100,000 reference gene copies.

Another method that can be used to determine methylation status after bisulfite treatment of a sample is Methylation Specific Nuclease-assisted Minor-allele Enrichment (MS-NaME) (see, e.g., Liu 2017 Nucleic Acids Res. 45(6):e39, which is herein incorporated by reference with respect to methods of determining methylation status). Ms-NaME is based on selective hybridization of probes to target sequences in the presence of DNA nuclease specific to double-stranded (ds) DNA (DSN), such that hybridization results in regions of double-stranded DNA that are subsequently digested by the DSN. Thus, oligonucleotide probes targeting unmethylated sequences generate local double stranded regions resulting to digestion of unmethylated targets; oligonucleotide probes capable of hybridizing to methylated sequences generate local double-stranded regions that result in digestion of methylated targets, leaving methylated targets intact. Moreover, oligonucleotide probes can direct DSN activity to multiple targets in bisulfite-treated DNA, simultaneously. Subsequent amplification can enrich non-digested sequences. Ms-NaME can be used, either independently or in combination with other techniques provided herein.

Another method that can be used to determine methylation status after bisulfite treatment of a sample is Methylation-sensitive Single Nucleotide Primer Extension (Ms-SNuPE™) (see, e.g., Gonzalgo 2007 Nat Protoc. 2(8):1931-6, which is herein incorporated by reference with respect to methods of determining methylation status). In Ms-SNuPE, strand-specific PCR is performed to generate a DNA template for quantitative methylation analysis using Ms-SNuPE. SNuPE is then performed with oligonucleotide(s) designed to hybridize immediately upstream of the CpG site(s) being interrogated. Reaction products can be electrophoresed on polyacrylamide gels for visualization and quantitation by phosphor-image analysis. Amplicons can also carry a directly or indirectly detectable labels such as a fluorescent label, radionuclide, or a detachable molecule fragment or other entity having a mass that can be distinguished by mass spectrometry. Detection may be carried out and/or visualized by means of, e.g., matrix assisted laser desorption/ionization mass spectrometry (MALDI) or using electron spray mass spectrometry (ESI).

Certain methods that can be used to determine methylation status after bisulfite treatment of a sample utilize a first oligonucleotide primer, a second oligonucleotide primer, and an oligonucleotide probe in an amplification-based method. For instance, the oligonucleotide primers and probe can be used in a method of real-time polymerase chain reaction (PCR) or droplet digital PCR (ddPCR). In various instances, the first oligonucleotide primer, the second oligonucleotide primer, and/or the oligonucleotide probe selectively hybridize methylated DNA and/or unmethylated DNA, such that amplification or probe signal indicate methylation status of a sample.

Other bisulfite-based methods for detecting methylation status (e.g., the presence of level of 5-methylcytosine) are disclosed, e.g., in Frommer (1992 Proc Natl Acad Sci USA. 1; 89(5):1827-31, which is herein incorporated by reference with respect to methods of determining methylation status).

In certain MSRE-qPCR embodiments, the amount of total DNA is measured in an aliquot of sample in native (e.g., undigested) form using, e.g., real-time PCR or digital PCR.

Various amplification technologies can be used alone or in conjunction with other techniques described herein for detection of methylation status. Those of skill in the art, having reviewed the present specification, will understand how to combine various amplification technologies known in the art and/or described herein together with various other technologies for methylation status determination known in the art and/or provided herein. Amplification technologies include, without limitation, PCR, e.g., quantitative PCR (qPCR), real-time PCR, and/or digital PCR. Those of skill in the art will appreciate that polymerase amplification can multiplex amplification of multiple targets in a single reaction. PCR amplicons are typically 100 to 2000 base pairs in length. In various instances, an amplification technology is sufficient to determine methylations status.

Digital PCR (dPCR) based methods involve dividing and distributing a sample across wells of a plate with 96-, 384-, or more wells, or in individual emulsion droplets (ddPCR) e.g., using a microfluidic device, such that some wells include one or more copies of template and others include no copies of template. Thus, the average number of template molecules per well is less than one prior to amplification. The number of wells in which amplification of template occurs provides a measure of template concentration. If the sample has been contacted with MSRE, the number of wells in which amplification of template occurs provides a measure of the concentration of methylated template.

In various embodiments a fluorescence-based real-time PCR assay, such as MethyLight™, can be used to measure methylation status (see, e.g., Campan 2018 Methods Mol Biol. 1708:497-513, which is herein incorporated by reference with respect to methods of determining methylation status). MethyLight is a quantitative, fluorescence-based, real-time PCR method to sensitively detect and quantify DNA methylation of candidate regions of the genome. MethyLight is uniquely suited for detecting low-frequency methylated DNA regions against a high background of unmethylated DNA, as it combines methylation-specific priming with methylation-specific fluorescent probing. Additionally, MethyLight can be combined with Digital PCR, for the highly sensitive detection of individual methylated molecules, with use in disease detection and screening.

Real-time PCR-based methods for use in determining methylation status typically include a step of generating a standard curve for unmethylated DNA based on analysis of external standards. A standard curve can be constructed from at least two points and can permit comparison of a real-time Ct value for digested DNA and/or a real-time Ct value for undigested DNA to known quantitative standards. In particular instances, sample Ct values can be determined for MSRE-digested and/or undigested samples or sample aliquots, and the genomic equivalents of DNA can be calculated from the standard curve. Ct values of MSRE-digested and undigested DNA can be evaluated to identify amplicons digested (e.g., efficiently digested; e.g., yielding a Ct value of 45). Amplicons not amplified under either digested or undigested conditions can also be identified. Corrected Ct values for amplicons of interest can then be directly compared across conditions to establish relative differences in methylation status between conditions. Alternatively or additionally, delta-difference between the Ct values of digested and undigested DNA can be used to establish relative differences in methylation status between conditions.

In certain particular embodiments, whole genome bisulfite sequencing among other techniques, can be used to determine the methylation status of a colorectal neoplasm (e.g., advanced adenoma, polyposis and/or colorectal cancer (e.g., early stage colorectal cancer)) methylation biomarker that is or includes a single methylation locus. In certain particular embodiments, whole genome bisulfite sequencing, among other techniques, can be used to determine the methylation status of a methylation biomarker that is or includes two or more methylation loci.

Those of skill in the art will further appreciate that methods, reagents, and protocols for whole genome bisulfite sequencing are well-known in the art. Unlike traditional whole genome sequencing, whole genome bisulfite sequencing is able to detect the methylation status of the cytosine nucleotide, due to deamination treatment with bisulfite reagent.

Those of skill in the art will appreciate that in embodiments in which a plurality of methylation loci (e.g., a plurality of DMRs) are analyzed for methylation status in a method of screening for colorectal cancer provided herein, methylation status of each methylation locus can be measured or represented in any of a variety of forms, and the methylation statuses of a plurality of methylation loci (preferably each measured and/or represented in a same, similar, or comparable manner) be together or cumulatively analyzed or represented in any of a variety of forms. In various embodiments, methylation status of each methylation locus can be measured as methylation portion. In various embodiments, methylation status of each methylation locus can be represented as the percentage value of methylated reads from total sequencing reads compared against reference sample. In various embodiments, methylation status of each methylation locus can be represented as a qualitative comparison to a reference, e.g., by identification of each methylation locus as hypermethylated or hypomethylated.

In some embodiments in which a single methylation locus is analyzed, hypermethylation of the single methylation locus constitutes a diagnosis that a subject is suffering from or possibly suffering from a condition (e.g., advanced adenoma, polyposis and/or colorectal cancer, e.g., early stage colorectal cancer), while absence of hypermethylation of the single methylation locus constitutes a diagnosis that the subject is likely not suffering from a condition. In some embodiments, hypermethylation of a single methylation locus (e.g., a single DMR) of a plurality of analyzed methylation loci constitutes a diagnosis that a subject is suffering from or possibly suffering from the condition, while the absence of hypermethylation at any methylation locus of a plurality of analyzed methylation loci constitutes a diagnosis that a subject is likely not suffering from the condition. In some embodiments, hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is suffering from or possibly suffering from the condition, while the absence of hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is not likely suffering from the condition. In some embodiments, hypermethylation of a determined number (e.g., a predetermined number) of methylation loci (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 DMRs) of a plurality of analyzed methylation loci (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 DMRs) constitutes a diagnosis that a subject is suffering from or possibly suffering from the condition, while the absence of hypermethylation of a determined number (e.g., a predetermined number) of methylation loci (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 DMRs) of a plurality of analyzed methylation loci (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 DMRs) constitutes a diagnosis that a subject is not likely suffering from the condition.

In some embodiments, methylation status of a plurality of methylation loci (e.g., a plurality of DMRs) is measured qualitatively or quantitatively and the measurement for each of the plurality of methylation loci are combined to provide a diagnosis. In some embodiments, the qualitative of quantitatively measured methylation status of each of a plurality of methylation loci is individually weighted, and weighted values are combined to provide a single value that can be comparative to a reference in order to provide a diagnosis.

### Applications

Methods and compositions of the present disclosure can be used in any of a variety of applications. For example, methods and compositions of the present disclosure can be used to screen, or aid in screening for a colorectal neoplasm, e.g., advanced adenoma, polyposis and/or colorectal cancer (e.g., early stage colorectal cancer). In various instances, screening using methods and compositions of the present disclosure can detect any stage of colorectal cancer, including without limitation early-stage colorectal cancer. In some embodiments, screening using methods and compositions of the present disclosure is applied to individuals 45 years of age or older, e.g., 45, 50, 55, 60, 65, 70, 75, 80, 85, or 90 years or older. In some embodiments, screening using methods and compositions of the present disclosure is applied to individuals 20 years of age or older, e.g., 20, 25, 30, 35, 40, 45, 50, 55, 60, 65, 70, 75, 80, 85, or 90 years or older. In some embodiments, screening using methods and compositions of the present disclosure is applied to individuals 20 to 50 years of age, e.g., 20 to 30 years of age, 20 to 40 years of age, 20 to 50 years of age, 30 to 40 years of age, 30 to 50 years of age, or 40 to 50 years of age. In various embodiments, screening using methods and compositions of the present disclosure is applied to individuals experiencing abdominal pain or discomfort, e.g., experiencing undiagnosed or incompletely diagnosed abdominal pain or discomfort. In various embodiments, screening using methods and compositions of the present disclosure is applied to individuals experiencing no symptoms likely to be associated with a colorectal neoplasm such as advanced adenoma, polyposis, and/or colorectal cancer. Thus, in certain embodiments, screening using methods and compositions of the present disclosure is fully or partially preventative or prophylactic, at least with respect to later or non-early stages of colorectal cancer.

In various embodiments, colorectal neoplasm screening using methods and compositions of the present disclosure can be applied to an asymptomatic human subject. As used herein, a subject can be referred to as “asymptomatic” if the subject does not report, and/or demonstrate by non-invasively observable indicia (e.g., without one, several, or all of device-based probing, tissue sample analysis, bodily fluid analysis, surgery, or colorectal cancer screening), sufficient characteristics of the condition to support a medically reasonable suspicion that the subject is likely suffering from the condition. Detection of a colorectal neoplasm such as advanced adenoma, polyposis and/or early stage colorectal cancer is particularly likely in asymptomatic individuals screened in accordance with methods and compositions of the present disclosure.

Those of skill in the art will appreciate that regular, preventative, and/or prophylactic screening for a colorectal neoplasm such as advanced adenoma and/or colorectal cancer improves diagnosis. As noted above, early stage cancers include, according to at least one system of cancer staging, Stages 0 to II C of colorectal cancer. Thus, the present disclosure provides, among other things, methods and compositions particularly useful for the diagnosis and treatment of colorectal neoplasms including advanced adenoma, polyposis and/or early stage colorectal cancer. Generally, and particularly in embodiments in which screening in accordance with the present disclosure is carried out annually, and/or in which a subject is asymptomatic at time of screening, methods and compositions of the present invention are especially likely to detect early stage colorectal cancer.

In various embodiments colorectal cancer screening in accordance with the present disclosure is performed once for a given subject or multiple times for a given subject. In various embodiments, colorectal cancer screening in accordance with the present disclosure is performed on a regular basis, e.g., every six months, annually, every two years, every three years, every four years, every five years, or every ten years.

In various embodiments, screening using methods and compositions disclosed herein will provide a diagnosis of a condition (e.g., a type or class of a colorectal neoplasm). In other instances, screening for colorectal neoplasms using methods and compositions disclosed herein will be indicative of having one or more conditions, but not definitive for diagnosis of a particular condition. For example, screening may be used to classify a subject as having one or more conditions or combination of conditions including, but not limited to, advanced adenoma, polyposis, and/or colorectal cancer. Screening may also be used to classify a subject as having a colorectal neoplasm without identifying which condition the subject has. In various instances, screening using methods and compositions of the present disclosure can be followed by a further diagnosis-confirmatory assay, which further assay can confirm, support, undermine, or reject a diagnosis resulting from prior screening, e.g., screening in accordance with the present disclosure.

As used herein, a diagnosis-confirmatory assay can be a colorectal cancer assay that provides a diagnosis recognized as definitive by medical practitioners, e.g., a colonoscopy-based diagnosed, or a colorectal cancer assay that substantially increases or decreases the likelihood that a prior diagnosis was correct, e.g., a diagnosis resulting from screening in accordance with the present disclosure. Diagnosis-confirmatory assays could include existing screening technologies, which are generally in need of improvement with respect to one or more of sensitivity, specificity, and non-invasiveness, particularly in the detection of early stage colorectal cancers.

In some instances, a diagnosis-confirmatory assay is a test that is or includes a visual or structural inspection of subject tissues, e.g., by colonoscopy. In some embodiments, colonoscopy includes or is followed by histological analysis. Visual and/or structural assays for colorectal cancer can include inspection of the structure of the colon and/or rectum for any abnormal tissues and/or structures. Visual and/or structural inspection can be conducted, for example, by use of a scope via the rectum or by CT-scan. In some instances, a diagnosis-confirmatory assay is a colonoscopy, e.g., including or followed by histological analysis. According to some reports, colonoscopy is currently the predominant and/or most relied upon diagnosis-confirmatory assay.

Another visual and/or structural diagnosis confirmatory assay based on computer tomography (CT) is CT colonography, sometimes referred to as virtual colonoscopy. A CT scan utilizes numerous x-ray images of the colon and/or rectum to produce dimensional representations of the colon. Although useful as a diagnosis-confirmatory assay, some reports suggest that CT colonography is not sufficient for replacement of colonoscopy, at least in part because a medical practitioner has not physically accessed the subject's colon to obtain tissue for histological analysis.

Another diagnosis-confirmatory assay can be a sigmoidoscopy. In sigmoidoscopy, a sigmoidoscope is used via the rectum to image portions of the colon and/or rectum. According to some reports, sigmoidoscopy is not widely used.

In some instances, a diagnosis-confirmatory assay is a stool-based assay. Typically, stool-based assays, when used in place of visual or structural inspection, are recommended to be utilized at a greater frequency than would be required if using visual or structural inspection. In some instances, a diagnosis-confirmatory assay is a guiac-based fecal occult blood test or a fecal immunochemical test (gFOBTs/FITs) (see, e.g., Navarro 2017 World J Gastroenterol. 23(20):3632-3642, which is herein incorporated by reference with respect to colorectal cancer assays). FOBTs and FITs are sometimes used for diagnosis of colorectal cancer (see, e.g., Nakamura 2010 J Diabetes Investig. October 19; 1(5):208-11, which is herein incorporated by reference with respect to colorectal cancer assays). FIT is based on detection of occult blood in stool, the presence of which is often indicative of colorectal cancer but is often not in sufficient volume to permit identification by the unaided eye. For example, in a typical FIT, the test utilizes hemoglobin-specific reagent to test for occult blood in a stool sample. In various instances, FIT kits are suitable for use by individuals in their own homes. When used in the absence of other diagnosis-confirmatory assays, FIT may be recommended for use on an annual basis. FIT is generally not relied upon to provide sufficient diagnostic information for conclusive diagnosis of colorectal cancer.

Diagnosis-confirmatory assays also include gFOBT, which is designed to detect occult blood in stool by chemical reaction. Like FIT, when used in the absence of other diagnosis-confirmatory assays, gFOBT may be recommended for use on an annual basis. gFOBT is generally not relied upon to provide sufficient diagnostic information for conclusive diagnosis of colorectal cancer.

Diagnosis-confirmatory assays can also include stool DNA testing. Stool DNA testing for colorectal cancer can be designed to identify DNA sequences characteristic of cancer in stool samples. When used in the absence of other diagnosis-confirmatory assays, stool DNA testing may be recommended for use every three years. Stool DNA testing is generally not relied upon to provide sufficient diagnostic information for conclusive diagnosis of colorectal cancer.

One particular screening technology is a stool-based screening test (Cologuard® (Exact Sciences Corporation, Madison, Wis., United States), which combines an FIT assay with analysis of DNA for abnormal modifications, such as mutation and methylation. The Cologuard® test demonstrates improved sensitivity as compared to FIT assay alone, but can be clinically impracticable or ineffective due to low compliance rates, which low compliance rates are at least in part due to subject dislike of using stool-based assays (see, e.g., doi: 10.1056/NEJMc1405215 (e.g., 2014 N Engl J Med. 371(2):184-188)). The Cologuard® test appears to leave almost half of the eligible population out of the screening programs (see, e.g., van der Vlugt 2017 Br J Cancer. 116(1):44-49). Use of screening as provided herein, e.g., by a blood-based analysis, would increase the number of individuals electing to screen for colorectal cancer (see, e.g., Adler 2014 BMC Gastroenterol. 14:183; Liles 2017 Cancer Treatment and Research Communications 10: 27-31). To present knowledge, only one existing screening technology for colorectal cancer, Epiprocolon, is FDA-approved and CE-IVD marked and is blood-based. Epiprocolon is based on hypermethylation of SEPT9 gene. The Epiprocolon test suffers from low accuracy for colorectal cancer detection with sensitivity of 68% and advanced adenoma sensitivity of only 22% (see, e.g., Potter 2014 Clin Chem. 60(9):1183-91). There is need in the art for, among other things, a non-invasive colorectal cancer screen that will likely achieve high subject adherence with high and/or improved specificity and/or sensitivity.

In various embodiments, screening in accordance with methods and compositions of the present disclosure reduces colorectal cancer mortality, e.g., by early colorectal cancer diagnosis. Data supports that colorectal cancer screening reduces colorectal cancer mortality, which effect persisted for over 30 years (see, e.g., Shaukat 2013 N Engl J Med. 369(12):1106-14). Moreover, colorectal cancer is particularly difficult to treat at least in part because colorectal cancer, absent timely screening, may not be detected until cancer is past early stages. For at least this reason, treatment of colorectal cancer is often unsuccessful. To maximize population-wide improvement of colorectal cancer outcomes, utilization of screening in accordance with the present disclosure can be paired with, e.g., recruitment of eligible subjects to ensure widespread screening.

In various embodiments, screening of colorectal neoplasms including one or more methods and/or compositions disclosed herein is followed by treatment of colorectal cancer, e.g., treatment of early stage colorectal cancer. In various embodiments, treatment of colorectal cancer, e.g., early stage colorectal cancer, includes administration of a therapeutic regimen including one or more of surgery, radiation therapy, and chemotherapy. In various embodiments, treatment of colorectal cancer, e.g., early stage colorectal cancer, includes administration of a therapeutic regimen including one or more of treatments provided herein for treatment of stage 0 colorectal cancer, stage I colorectal cancer, and/or stage II colorectal cancer.

In various embodiments, treatment of colorectal cancer includes treatment of early stage colorectal cancer, e.g., stage 0 colorectal cancer or stage I colorectal cancer, by one or more of surgical removal of cancerous tissue e.g., by local excision (e.g., by colonoscope), partial colectomy, or complete colectomy.

In various embodiments, treatment of colorectal cancer includes treatment of early stage colorectal cancer, e.g., stage II colorectal cancer, by one or more of surgical removal of cancerous tissue (e.g., by local excision (e.g., by colonoscope), partial colectomy, or complete colectomy), surgery to remove lymph nodes near to identified colorectal cancer tissue, and chemotherapy (e.g., administration of one or more of 5-FU and leucovorin, oxaliplatin, or capecitabine).

In various embodiments, treatment of colorectal cancer includes treatment of stage III colorectal cancer, by one or more of surgical removal of cancerous tissue (e.g., by local excision (e.g., by colonoscopy-based excision), partial colectomy, or complete colectomy), surgical removal of lymph nodes near to identified colorectal cancer tissue, chemotherapy (e.g., administration of one or more of 5-FU, leucovorin, oxaliplatin, capecitabine, e.g., in a combination of (i) 5-FU and leucovorin, (ii) 5-FU, leucovorin, and oxaliplatin (e.g., FOLFOX), or (iii) capecitabine and oxaliplatin (e.g., CAPEOX)), and radiation therapy.

In various embodiments, treatment of colorectal cancer includes treatment of stage IV colorectal cancer, by one or more of surgical removal of cancerous tissue (e.g., by local excision (e.g., by colonoscope), partial colectomy, or complete colectomy), surgical removal of lymph nodes near to identified colorectal cancer tissue, surgical removal of metastases, chemotherapy (e.g., administration of one or more of 5-FU, leucovorin, oxaliplatin, capecitabine, irinotecan, VEGF-targeted therapeutic agent (e.g., bevacizumab, ziv-aflibercept, or ramucirumab), EGFR-targeted therapeutic agent (e.g., cetuximab or panitumumab), Regorafenib, trifluridine, and tipiracil, e.g., in a combination of or including (i) 5-FU and leucovorin, (ii) 5-FU, leucovorin, and oxaliplatin (e.g., FOLFOX), (iii) capecitabine and oxaliplatin (e.g., CAPEOX), (iv) leucovorin, 5-FU, oxaliplatin, and irinotecan (FOLFOXIRI), and (v) trifluridine and tipiracil (Lonsurf)), radiation therapy, hepatic artery infusion (e.g., if cancer has metastasized to liver), ablation of tumors, embolization of tumors, colon stent, colorectomy, colostomy (e.g., diverting colostomy), and immunotherapy (e.g., pembrolizumab).

Those of skill in the art that treatments of colorectal cancer provided herein can be utilized, e.g., as determined by a medical practitioner, alone or in any combination, in any order, regimen, and/or therapeutic program. Those of skill in the art will further appreciate that advanced treatment options may be appropriate for earlier stage cancers in subjects previously having suffered a cancer or colorectal cancer, e.g., subjects diagnosed as having a recurrent colorectal cancer.

In some embodiments, methods and compositions for colorectal neoplasm screening provided herein can inform treatment and/or payment (e.g., reimbursement for or reduction of cost of medical care, such as screening or treatment) decisions and/or actions, e.g., by individuals, healthcare facilities, healthcare practitioners, health insurance providers, governmental bodies, or other parties interested in healthcare cost.

In some embodiments, methods and compositions for colorectal neoplasm screening provided herein can inform decision making relating to whether health insurance providers reimburse a healthcare cost payer or recipient (or not), e.g., for (1) screening itself (e.g., reimbursement for screening otherwise unavailable, available only for periodic/regular screening, or available only for temporally- and/or incidentally-motivated screening); and/or for (2) treatment, including initiating, maintaining, and/or altering therapy, e.g., based on screening results. For example, in some embodiments, methods and compositions for colorectal neoplasm screening provided herein are used as the basis for, to contribute to, or support a determination as to whether a reimbursement or cost reduction will be provided to a healthcare cost payer or recipient. In some instances, a party seeking reimbursement or cost reduction can provide results of a screen conducted in accordance with the present specification together with a request for such reimbursement or cost reduction of a healthcare cost. In some instances, a party making a determination as to whether or not to provide a reimbursement or cost reduction of a healthcare cost will reach a determination based in whole or in part upon receipt and/or review of results of a screen conducted in accordance with the present specification.

For the avoidance of any doubt, those of skill in the art will appreciate from the present disclosure that methods and compositions for colorectal cancer diagnosis of the present specification are at least for in vitro use. Accordingly, all aspects and embodiments of the present disclosure can be performed and/or used at least in vitro.

### Kits

The present disclosure includes, among other things, kits including one or more compositions for use in screening as provided herein, optionally in combination with instructions for use thereof in screening (e.g., screening for a colorectal cancer neoplasm, e.g., advanced adenoma and/or colorectal cancer (e.g., early-stage colorectal cancer)). In various embodiments, a kit for screening for colorectal neoplasms can include one or more oligonucleotide capture baits (e.g., one or more biotinylated oligonucleotide probes). In certain embodiments, the kit for screening optionally includes one or more bisulfite reagents as disclosed herein. In certain embodiments, the kit for screening optionally includes one or more enzymatic conversion reagents as disclosed herein.

Oligonucleotide capture baits are useful in next generation sequencing (NGS) techniques to target particular regions of interest of DNA. In certain embodiments, one or more capture baits are targeted to capture a region of interest of the DNA corresponding to one or more methylation loci (e.g., methylation loci comprising at least a portion of one or more DMRs, e.g., as found in Tables 1, 5, and/or 6). Oligonucleotide capture baits are intended to enrich the target DNA region, and aid in preparation of a DNA library. The enriched target region will then be sequenced using, for example, an NGS sequencing technique as discussed herein.

In various embodiments, a kit for screening can include one or more of: one or more oligonucleotide primers (e.g., one or more oligonucleotide primer pairs), one or more MSREs, one or more reagents for qPCR (e.g., reagents sufficient for a complete qPCR reaction mixture, including without limitation dNTP and polymerase), and instructions for use of one or more components of the kit for colorectal neoplasm screening. In various embodiments, a kit for screening of colorectal neoplasms can include one or more of: one or more oligonucleotide primers (e.g., one or more oligonucleotide primer pairs), one or more bisulfite reagents, one or more reagents for qPCR (e.g., reagents sufficient for a complete qPCR reaction mixture, including without limitation dNTP and polymerase), and instructions for use of one or more components of the kit for colorectal cancer screening.

In certain embodiments, a kit of the present disclosure includes at least one oligonucleotide primer pair for amplification of a methylation locus and/or DMR as disclosed herein (e.g., in Tables 1, 5, and/or 6).

In some instances, a kit of the present disclosure includes one or more oligonucleotide primer pairs for amplification of one or more methylation loci of the present disclosure. In some instances, a kit of the present disclosure includes one or more oligonucleotide primer pairs for amplification of one or more methylation loci that are or include all or a portion of one or more genes identified in Table 1. In some particular instances, a kit of the present disclosure includes oligonucleotide primer pairs for a plurality of methylation loci that each are or include all or a portion of a gene identified in Table 1, the plurality of methylation loci including, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 methylation loci, e.g., as provided in Tables 1, 5, and/or 6.

In some instances, a kit of the present disclosure includes one or more oligonucleotide primer pairs for amplification of one or more DMRs of the present disclosure. In some instances, a kit of the present disclosure includes one or more oligonucleotide primer pairs for amplification of one or more DMRs that are, include all or a portion of, or are within a gene identified in Table 1. In some instances, a kit of the present disclosure includes one or more oligonucleotide primer pairs for amplification of one or more DMRs that are not associated with a presently known gene. In some particular embodiments, a kit of the present disclosure includes oligonucleotide primer pairs for a plurality of DMRs, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 DMRs, e.g., as provided in Tables 1, 5, and/or 6.

A kit of the present disclosure can further include one or more MSREs individually or in a single solution. In various embodiments, one or more MSREs are selected from the set of MSREs including AciI, Hin6I, HpyCH4IV, and HpaII (e.g., such that the kit includes AciI, Hin6I, and HpyCH4IV, either individually or in a single solution). In certain embodiments, a kit of the present disclosure includes one or more reagents for qPCR (e.g., reagents sufficient for a complete qPCR reaction mixture, including without limitation dNTP and polymerase).

### EXAMPLES

### Example 1. Identification of Markers Associated with Colorectal Neoplasms

The present example includes identification of markers relevant to diagnosis of and/or classification of colorectal neoplasms (e.g., colorectal cancer, advanced adenomas, polyposis). This identification is performed using whole genome bisulfite sequencing (WGBS) of genomic DNA (gDNA) samples obtained from tissues and blood components as described herein. As also explained herein, whole genome bisulfite sequencing (WGBS) allows for determinations to be made regarding the methylation status of various loci within gDNA. The difference in methylation status between a loci in gDNA obtained from a tissue sample from a subject suffering from a colorectal neoplasm as compared to the same loci in gDNA from a control subject is indicative of a differentially methylated region (DMR) that may be useful in determining whether or not a subject has a colorectal neoplasm.

Furthermore, identified DMRs may be additionally useful in classification of diagnosed colorectal neoplasm. The identification of DMRs through WGBS also allows for further development of more targeted kits and assays for the determination of methylation status. For example, methylation status could be determined using quantitative polymerase chain reaction (qPCR), methylation sensitive restriction enzyme quantitative polymerase chain reaction (MSRE-qPCR), targeted next generation sequencing (NGS) techniques, or other equivalent techniques as would be understood by those of skill in the art.

Tissue samples with different histological background as well as buffy coat samples were used in order to obtain gDNA for whole genome bisulfite sequencing. Sources from which DNA was obtained can be seen below in Table 2. Samples derived from advanced adenoma colonic tissue were considered to be positive for the condition. Other tissue and sample types were considered to be controls against which methylation status of the advanced adenoma tissue was evaluated. Other tissue types (“other control”) included tissue samples from other organs of the body such as lungs, breasts, pancreas, and stomach. Other cancers included tissue samples of cancerous tissue from other organs of the body including breast cancer, lung cancer, pancreatic cancer, and stomach cancer. Buffy coat samples included buffy coat extracted from the blood of 2 patients with advanced adenoma and 19 patients with non-neoplastic diseases.

TABLE 2
Analysis sample sources.

| | Buffy | Normal | Pathological |
|---|---|---|---|
| Patient type | Coat | tissue | tissue |
| Colorectal Adenoma | 2 | 57 | 90 |
| Breast cancer | | 4 | 10 |
| Lung cancer | | 10 | 10 |
| Pancreas cancer | | 9 | 10 |
| Stomach cancer | | 11 | 12 |
| Small intestine cancer | | 2 | |
| Colorectal cancer | | 85 | |
| Non-neoplastic | 19 | | 1 |

Genomic DNA (gDNA) from tissue and buffy coat samples was extracted using a DNeasy Blood & Tissue kit (Qiagen) according to a protocol from the manufacturer. Extracted gDNA was then processed in order to fragment it. gDNA was fragmented into segments having lengths of about 400 bp with a Covaris 5220 ultrasonicator. Exemplary settings of the ultrasonicator used for fragmentation of gDNA were as follows: peak incident power 140; Duty factor 5%; Cycles per burst 200; and a treatment time of 55s.

The extracted and shredded gDNA (genomic DNA) was bisulfite-converted with EZ DNA Methylation-Lightning kit (ZymoResearch). Sequencing libraries were prepared from the bisulfite converted DNA by using Accel-NGS Methyl-seq DNA library kit (Swift Biosciences) and consequently sequenced with average depth of 37.5× with NovaSeq6000 (Illumina) equipment, using paired-end sequencing (2×150 bp). The sequenced reads were aligned to a bisulfite-converted human genome (Ensembl 91 assembly) using Bisulfite Read Mapper with Bowtie 2. The following steps were used to align sequenced reads to a bisulfite-converted human genome:

Evaluation of the sequencing quality

Alignment to a reference genome (hG38)

Deduplication and cleaning from adapter dimers

Methylation calling (e.g., identification of methylated nucleic acids)

The DMRSeq analysis package was used to identify differentially methylated regions of DNA of advanced adenoma tissue as compared to other control samples (e.g., healthy colon tissue, tissues from other cancers, other control tissues, and buffy coat samples). The q-value of the region, which is the p-value corrected with a between-group label permutation test, was evaluated in order to select for regions of DNA from subjects with advanced adenoma which were significantly differently methylated from the same region in DNA obtained from a control subject. 69740 regions were found to have a q-value less than 0.05. A q-value being less than 0.05 is indicative of high statistical significance of the differentially methylated region.

Percentage methylation matrices were built for CpGs found inside each differently methylated region. At this stage, the percentage methylation is calculated per CpG position. For each sample, the percentage methylation is calculated for a particular CpG within a DMR by calculating the percentage of methylated reads (“M”) from the total read count (M+UM, where “UM” is the number of unmethylated reads). Each strata was separated using a subset of the full data as shown below in Table 3.

TABLE 3
Data subsets.

| Colorectal | Colorectal | Colorectal |
|---|---|---|
| Adenoma_PAT | Adenoma_NORM | cancer_NORM |
| 57 | 44 | 33 |
| Lung cancer_NORM | Gastric cancer_PAT | Gastric_NORM |
| 10 | 12 | 11 |
| Breast cancer_PAT | Breast cancer_NORM | Lung cancer_PAT |
| 10 | 4 | 10 |
| Pancreatic | Pancreatic | Buffy coat |
| cancer_PAT | cancer_NORM | |
| 10 | 9 | 21 |

Mean (μ) and standard deviation (σ) of the percentage methylation (e.g., the percent of CpGs methylated per DMR) of each DMR was calculated vector-wise, without considering the information of the order (e.g., position, location) of the CpGs within the DMR. That is, take the percentage methylation matrix (rows are CpGs, columns are samples) and convert it to a vector, and then compute mean and standard deviation. This metric is computed for each strata separately, meaning it was calculated for the advanced adenoma group and for each sub-group in the control group, i.e. between samples from “Colorectal Adenoma_PAT” and all the sub-groups of non “Colorectal Adenoma_PAT” as in Table 3. This information was used as filtering criteria for next step.

Further filtering was done to select regions that were not highly methylated in control samples as compared to advanced adenoma samples. Regions that were found to have μ_{controls}<0.2 (a mean methylation percentage less than 20%) in control samples (e.g., all non-advanced adenoma samples) were used. For assuring low percentage methylation of a region in all control samples, an upper-bound (u_{pperbound}) for the standard deviation (σ_{controls}) was defined to be E(σ_{controls, μcontrols}<0.2), i.e. the mean of the standard deviation in controls using only the regions that fulfill μ_{controls}<0.2.

Finally, regions were selected whose effect size (β) of the difference between advanced adenoma and all the rest of the tissue samples is sufficiently large, having values for β being less than −0.4. 54 DMRs were identified in Table 1 below that fulfilled all pre-defined criteria of μ_{controls}<0.2 and β<−0.4.

TABLE 1
List of DMRs found to have significantly altered methylation pattern(s) in the blood
of colorectal cancer and/or advanced adenoma patients compared to controls.

| | | | | | | Size |
|---|---|---|---|---|---|---|
| | | | Associated | SEQ | | of the |
| Chr | Start | End | Genes | ID NO: | uid | region |
| 1 | 34165443 | 34165675 | CSMD2, | 1 | 1_34165443_34165675 | 233 |
| | | | C1orf94 | | | |
| 1 | 219928356 | 219928386 | SLC30A10 | 2 | 1_219928356_219928386 | 31 |
| 1 | 161539915 | 161540181 | FCGR2A | 3 | 1_161539915_161540181 | 267 |
| 1 | 150294178 | 150294265 | MRPS21 | 4 | 1_150294178_150294265 | 88 |
| 2 | 47569627 | 47569858 | MSH2, KCNK12 | 5 | 2_47569627_47569858 | 232 |
| 3 | 157437029 | 157437296 | VEPH1, PTX3 | 6 | 3_157437029_157437296 | 268 |
| 3 | 173397284 | 173397387 | NLGN1 | 7 | 3_173397284_173397387 | 104 |
| 3 | 43998153 | 43998214 | NA | 8 | 3_43998153_43998214 | 62 |
| 3 | 10993689 | 10993900 | SLC6A1 | 9 | 3_10993689_10993900 | 212 |
| 3 | 181712580 | 181712614 | SOX2, | 10 | 3_181712580_181712614 | 35 |
| | | | SOX2-OT | | | |
| 3 | 180679674 | 180679706 | CCDC39, | 11 | 3_180679674_180679706 | 33 |
| | | | LOC101928882 | | | |
| 7 | 65252405 | 65252749 | NA | 12 | 7_65252405_65252749 | 345 |
| 7 | 64314242 | 64314346 | ZNF736 | 13 | 7_64314242_64314346 | 105 |
| 8 | 53877760 | 53878892 | RGS20 | 14 | 8_53877760_53878892 | 1133 |
| 8 | 66177597 | 66177765 | CRH | 15 | 8_66177597_66177765 | 169 |
| 8 | 108787618 | 108787717 | TMEM74 | 16 | 8_108787618_108787717 | 100 |
| 8 | 53881543 | 53881774 | RGS20 | 17 | 8_53881543_53881774 | 232 |
| 4 | 183905237 | 183905728 | STOX2 | 18 | 4_183905237_183905728 | 492 |
| 4 | 183904880 | 183905140 | STOX2 | 19 | 4_183904880_183905140 | 261 |
| 4 | 13545274 | 13545403 | NKX3-2 | 20 | 4_13545274_13545403 | 130 |
| 4 | 127623100 | 127623235 | INTU | 21 | 4_127623100_127623235 | 136 |
| 4 | 30718010 | 30718076 | PCDH7 | 22 | 4_30718010_30718076 | 67 |
| 4 | 82562331 | 82562477 | TMEM150C | 23 | 4_82562331_82562477 | 147 |
| 5 | 88660609 | 88660834 | LINC00461 | 24 | 5_88660609_88660834 | 226 |
| 5 | 83474370 | 83474485 | VCAN | 25 | 5_83474370_83474485 | 116 |
| 5 | 79512985 | 79513164 | HOMER1 | 26 | 5_79512985_79513164 | 180 |
| 5 | 51387638 | 51388080 | LOC642366, | 27 | 5_51387638_51388080 | 443 |
| | | | ISL1 | | | |
| 6 | 129767354 | 129767459 | NA | 28 | 6_129767354_129767459 | 106 |
| 6 | 27288050 | 27288544 | NA | 29 | 6_27288050_27288544 | 495 |
| 6 | 165309346 | 165309582 | C6orf118 | 30 | 6_165309346_165309582 | 237 |
| 6 | 6320205 | 6320663 | F13A1 | 31 | 6_6320205_6320663 | 459 |
| 6 | 68635305 | 68635560 | ADGRB3, | 32 | 6_68635305_68635560 | 256 |
| | | | LOC101928307 | | | |
| 6 | 27670532 | 27670614 | NA | 33 | 6_27670532_27670614 | 83 |
| 6 | 1524106 | 1524152 | NA | 34 | 6_1524106_1524152 | 47 |
| 11 | 79438095 | 79438308 | TENM4 | 35 | 11_79438095_79438308 | 214 |
| 11 | 94740046 | 94740862 | LOC105369438, | 36 | 11_94740046_94740862 | 817 |
| | | | AMOTL1 | | | |
| 13 | 93228702 | 93229444 | GPC6 | 37 | 13_93228702_93229444 | 743 |
| 13 | 94712995 | 94713032 | SOX21, | 38 | 13_94712995_94713032 | 38 |
| | | | SOX21-AS1 | | | |
| 13 | 67231493 | 67231965 | PCDH9 | 39 | 13_67231493_67231965 | 473 |
| 13 | 61414743 | 61414845 | PCDH20 | 40 | 13_61414743_61414845 | 103 |
| 9 | 132579614 | 132579683 | BARHL1 | 41 | 9_132579614_132579683 | 70 |
| 10 | 22475853 | 22476043 | NA | 42 | 10_22475853_22476043 | 191 |
| 10 | 117165171 | 117165431 | MIR3663HG | 43 | 10_117165171_117165431 | 261 |
| 10 | 16521408 | 16521447 | C1QL3 | 44 | 10_16521408_16521447 | 40 |
| 12 | 78865146 | 78865306 | SYT1 | 45 | 12_78865146_78865306 | 161 |
| 12 | 15221303 | 15221461 | RERG | 46 | 12_15221303_15221461 | 159 |
| 12 | 24563707 | 24564068 | SOX5 | 47 | 12_24563707_24564068 | 362 |
| 12 | 53974529 | 53974571 | HOXC11, | 48 | 12_53974529_53974571 | 43 |
| | | | HOTAIR | | | |
| 14 | 59870236 | 59870262 | RTN1 | 49 | 14_59870236_59870262 | 27 |
| 18 | 27184277 | 27184863 | AQP4-AS1, | 50 | 18_27184277_27184863 | 587 |
| | | | CHST9 | | | |
| 20 | 9516555 | 9516632 | LAMP5-AS1, | 51 | 20_9516555_9516632 | 78 |
| | | | LAMP5 | | | |
| 21 | 31344104 | 31344160 | TIAM1 | 52 | 21_31344104_31344160 | 57 |
| 19 | 20052466 | 20053193 | NA | 53 | 19_20052466_20053193 | 728 |
| X | 625391 | 625465 | SHOX | 54 | X_625391_625465 | 75 |

### Example 2. Verification of Selected Differentially Methylated Regions on Plasma Samples

The present example includes experimental verification that DMRs (differently methylated regions) could be identified using plasma samples from subjects using whole genome bisulfate sequencing (WGBS). For screening purposes, it is important to be able to use readily obtainable samples, such as blood, urine, or stool in order to facilitate easy determinations of colorectal neoplasia. Prior to the experiment discussed herein, it was not known whether the colorectal neoplasia biomarkers found in Example 1 could be sufficiently analyzed from cfDNA to successfully capture the ctDNA portion that allows for identifying and/or classifying subjects and/or samples as being positive for a colorectal neoplasia.

The following describes an exemplary extraction method for cell free DNA (cfDNA) from plasma of subjects. 20 mL of plasma was collected from 33 participants attending colorectal cancer screening centers and oncology clinics in Spain during 2018-2019. Samples were obtained from subjects having advanced adenoma, colorectal cancer, polyposis, or control subjects who were not diagnosed as having a colorectal neoplasm. The breakdown of the sample cohort is further described below in Table 4.

TABLE 4
Plasma sample cohort breakdown.

| | Adavnced | Colorectal | | |
|---|---|---|---|---|
| | Adenoma | cancer | Polyposis | Control |
| | n = 10 | n = 11 | n = 2 | n = 10 |
| Characteristics | | | | |
| Age (years, | 64.5 (52-71) | 65.6 (52-77) | 65.5 (60-71) | 67.6 (53-76) |
| average (IQR)) | | | | |
| Female | 5 | 6 | 1 | 5 |
| Male | 5 | 5 | 1 | 5 |
| Stage | | | | |
| Stage 0 | | 2 | | |
| Stage I | | 3 | | |
| Stage II | | 3 | | |
| Stage III | | 3 | | |
| Adenoma | | | | |
| characteristics | | | | |
| High grade | 5 | | | |
| dysplasia | | | | |
| >=10 mm | 5 | | | |
| Tubular | 4 | | | |
| Tubulovillous | 4 | | | |
| Serrated | 2 | | | |

cfDNA (cell free DNA) was extracted from 20 ml of plasma samples with QIAamp® Circulating Nucleic Acid kit (Qiagen) following a manufacturer's protocol. Subsequently, extracted cfDNA was directly bisulfite converted with an EZ DNA Methylation-Lightning kit (ZymoResearch).

Library preparation was performed using the Accel-NGS Methyl-seq DNA library kit (Swift Biosciences). In brief, around 25 ng of bisulfite-converted cfDNA from each sample was denatured. A small tail with a first truncated adapter (“truncated adapter 1”) was added to the 3′ end of the DNA fragments. Following a primer extension reaction for the synthesis of the complementary bottom DNA strand, a second truncated adapter (“truncated adapter 2”) was ligated to the complementary bottom DNA strand. Then, qPCR was performed in order to determine an optimal PCR cycle number. Through indexing PCR, full length adapters for dual-indexing were incorporated and the yield increased. A clean-up of the adaptor-ligated DNA fragments was performed by Agencourt AMPure XP beads (Beckman Coulter), and the DNA was eluted in low EDTA (ethylene-diamine-tetraacetic acid) TE buffer.

Size selection and efficient removal of small fragments, such as adaptor dimers, was performed through gel electrophoresis using 2% agarose gels (Thermofisher) and subsequent excision of DNA fragments ranging between approximately 280 to 320 base pairs. DNA was eluted from the agarose gels with the Zymoclean Gel DNA Recovery kit (ZymoResearch) in RNase-DNase-free water.

Sequencing was performed on an Illumina Novaseq 6000 PE150 with 25% PhiX per lane.

The sequenced reads were aligned to a bisulfite-converted human genome (Ensembl 91 assembly), using Bisulfite Read Mapper with Bowtie 2 following these steps:

Evaluation the sequencing quality

Alignment to a reference genome (hG38)

Deduplication and cleaning from adapter dimers

Methylation calling

All 54 DMRs from Table 1 were evaluated in plasma samples from subjects as shown above in Table 4. By evaluating all 54 DMRs of Table 1, an AUC of 90% was achieved for separating advanced adenomas from controls. This result confirms that of all these regions are useful and contribute to advanced adenoma detection in plasma.

However, only a small subset of DMRs are required to detect advanced adenomas. Further analysis shows that Combination 1, which uses four DMR regions as can be seen in Table 5 below, gives surprisingly good separation between advanced adenomas and control samples. The four DMR regions of Table 5 are also provided with alternative identifiers by which the DMR may be identified definitively identified based on a gene associated with the DMR (if available) and the last three digits of the start position. A second identifier is provided which identifies the DMR based on the chromosome number the DMR is found on along with the start and end positions of the DMR.

TABLE 5
Combination 1 of four DMRs.

| | chr | start | end | Alternative Identifier 1 | Alternative Identifier 2 |
|---|---|---|---|---|---|
| SLC6A1 | 3 | 10993689 | 10993900 | SLC6A1 ′689 | chr3: 10993689-10993900 |
| NA | 19 | 20052466 | 20053193 | | chr19: 20052466-20053193 |
| F13A1 | 6 | 6320205 | 6320663 | F13A1 ′205 | chr6: 6320205-6320663 |
| BARHL1 | 9 | 132579614 | 132579683 | BARHL1 ′614 | chr9: 132579614-132579683 |

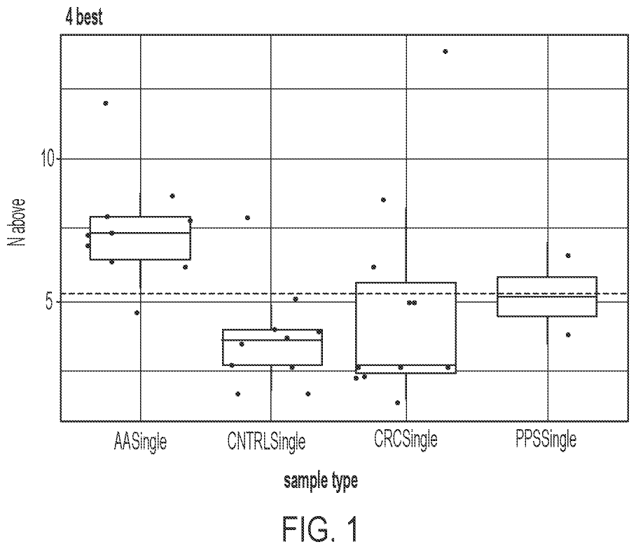

Combination 1 is able to distinguish between samples of subjects having advanced adenomas and control subjects with 90% (9/10) sensitivity at 90% (9/10) specificity. Combination 1 was also able to be used to distinguish between control patients and other colorectal neoplasms. For example when using Combination 1, sensitivity for polyposis was at 50%, whereas the sensitivity for colorectal cancer detection was 27%. A box-plot presentation of the separation of patients based on the 4-region combination can be seen in FIG. 1. In FIG. 1, the following abbreviations are used: AA stands for advanced adenoma, CNTRL for control, CRC for colorectal cancer, and PPS for polyposis. The term “single” used in FIG. 1 indicates that samples from individual subjects, as opposed to pooled samples, are used in the experiment. The term “N above” (the y-axis) refers to the number of reads that are above the mean (μ) methylation calculated in advanced adenoma group.

A second combination of four DMRs shown in Table 6 (Combination 2) is also useful in detecting colorectal neoplasms.

TABLE 6
Combination 2 of four DMRs.

| | chr | start | end | Alternative Identifier 1 | Alternative Identifier 2 |
|---|---|---|---|---|---|
| CSMD2 | 1 | 34165443 | 34165675 | CSMD2 ′443 | chr1: 34165443-34155675 |
| SLC6A1 | 3 | 10993689 | 10993900 | SLC6A1 ′689 | chr3: 10993689-10993900 |
| NA | 6 | 27670532 | 27670614 | | chr6: 27670532-27670614 |
| NA | 19 | 20052466 | 20053193 | | chr19: 20052466-20053193 |

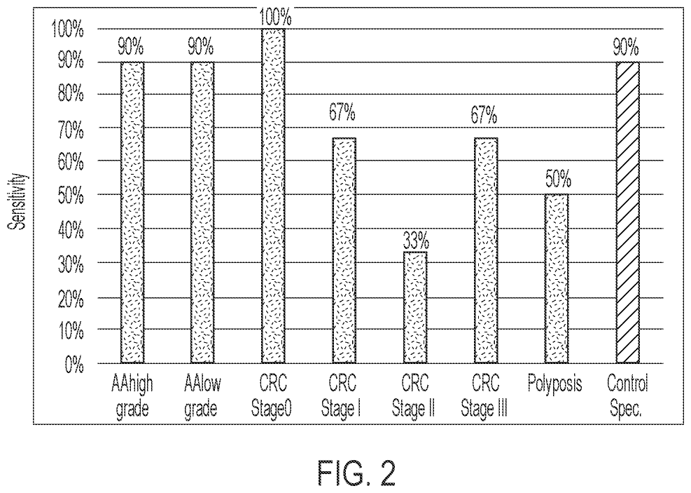

Combination 2 was used to detect advanced adenoma with 80% sensitivity, polyposis with 50% sensitivity, and 54.5% sensitivity for colorectal cancer. When looking at the sub-classifications of the colorectal cases, Combination 2 preferentially detects stage 0 and stage I colorectal cancers (CRCs) as seen in FIG. 2. Both high grade and low grade dysplasia adenomas were separated with equal sensitivity, further enforcing that this combination of markers is especially useful for early detection.

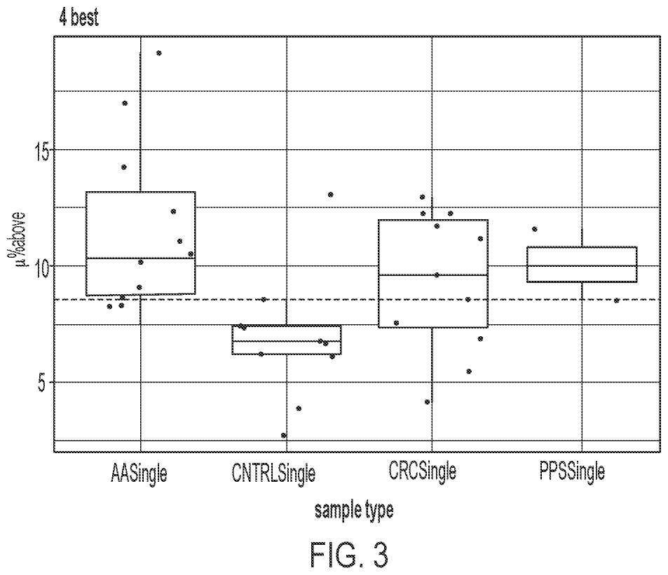

A box-plot presentation of the separation of patients based on Combination 2 can be seen in FIG. 3. The term “single” used in FIG. 3 indicates that samples from individual subjects, as opposed to pooled samples, are used in the experiment. The term “μ % above” (the y-axis) refers to the percentage of reads that are above the mean (μ) methylation calculated in advanced adenoma group.

Lowering the specificity to 80% would assure the correct classification of 100% of patients with advanced adenomas, 100% of patients with polyposis, and 64% of the colorectal cancer patients. These results indicate once again the ability to use this combination of four DMRs for detection and classification of colorectal neoplasia.

Statistical results of individual DMRs in each of Combinations 1 and 2 are presented below in Tables 7 and 8, respectively, for subjects having advanced adenomas (AAs).

TABLE 7
Individual DMR performance for DMRs in Combination 1 in
advanced adenoma vs controls based on the “μ% above” value.
Combination1

| | chr | start | end | Sensitivity | Specificity |
|---|---|---|---|---|---|
| SLC6A1 | 3 | 10993689 | 10993900 | 60% | 90% |
| NA | 19 | 20052466 | 20053193 | 40% | 90% |
| F13A1 | 6 | 6320205 | 6320663 | 10% | 90% |
| BARHL1 | 9 | 132579614 | 132579683 | 30% | 90% |

TABLE 8
Individual DMR performance for regions in Combination 2 in
advanced adenoma vs controls based on the “μ% above” value
Combination2

| | chr | start | end | Sensitivity | Specificity |
|---|---|---|---|---|---|
| CSMD2 | 1 | 34165443 | 34165675 | 30% | 90% |
| SLC6A1 | 3 | 10993689 | 10993900 | 60% | 90% |
| NA | 6 | 27670532 | 27670614 | 60% | 90% |
| NA | 19 | 20052466 | 20053193 | 40% | 90% |

It can be seen from the above tables that two DMRs (SLC6A1 '689 and chr6: 27670532-27670614) reached to 60% sensitivity at 90% specificity. However, combinations of DMRs were able to achieve improved classification and detection of advanced adenomas over individual DMRs used alone. In Combination 1, advanced adenoma detection and classification achieved 90% sensitivity at 90% specificity. In Combination 2, advanced adenoma detection and classification achieved 80% sensitivity at 90% specificity. Additionally, both Combinations 1 and 2 share SLC6A1 '689 and chr19:20052466-200553193 in common, which indicates their importance in classifying and detecting advanced adenomas. The improvement of detection of advanced adenoma with multiple DMRs attests to the complexity of advanced adenoma and other colorectal neoplasms.

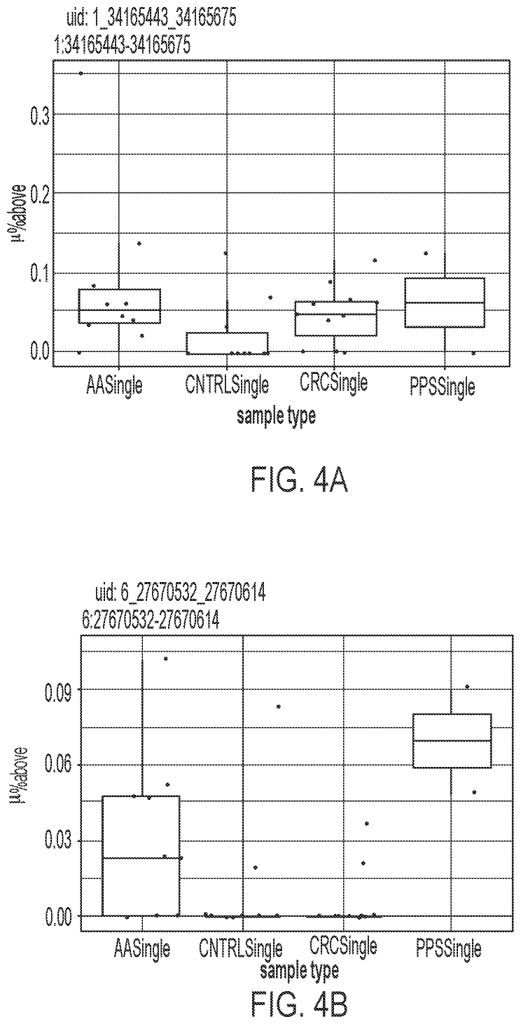

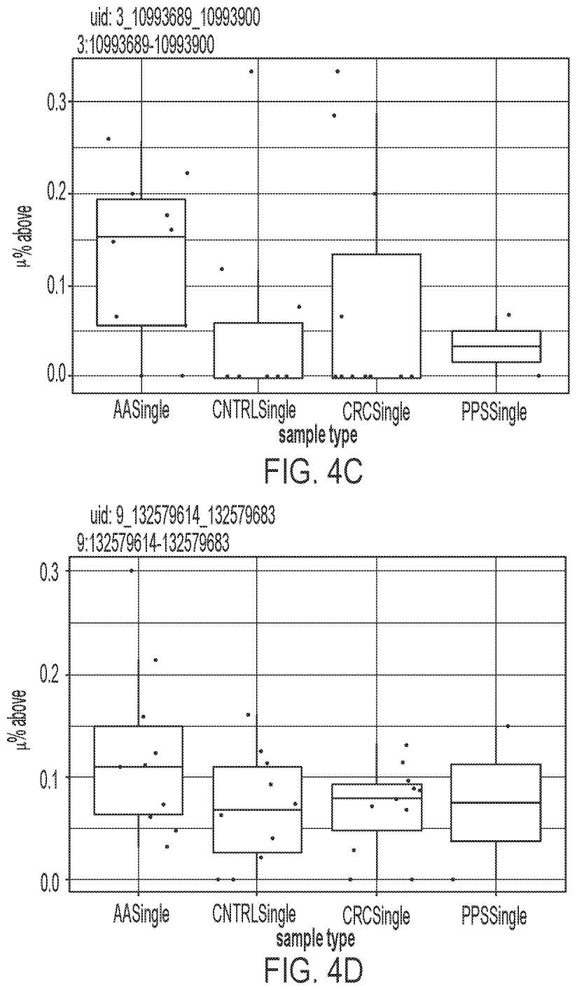

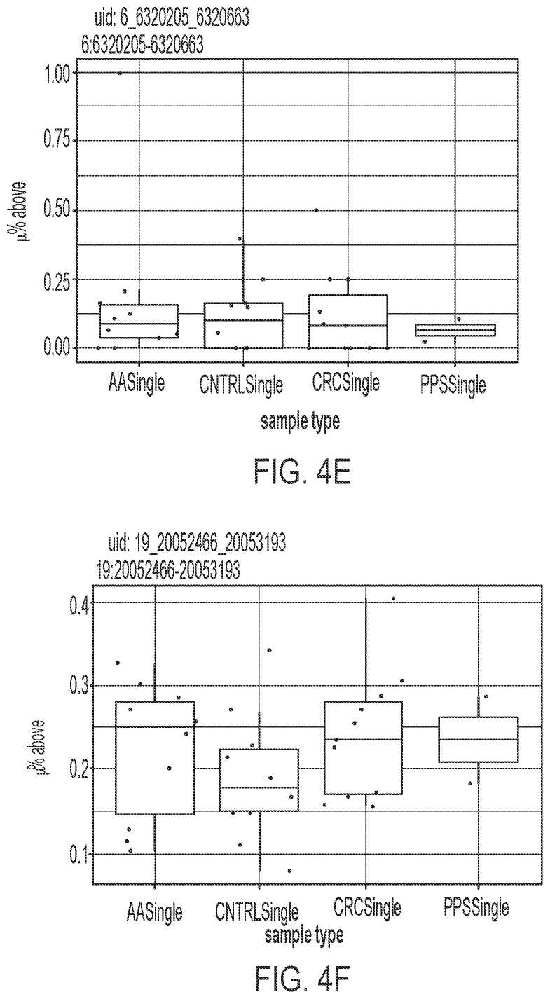

Individual boxplots representing percent of reads above mean (μ) methylation calculated in advanced adenoma group of each DMR in Combinations 1 and 2 are shown in FIGS. 4A-F. The percent of reads above mean methylation of the region in advanced adenoma is shown for each subject classified as having advanced adenomas (AA), colorectal cancer (CRC) or polyposis (PPS) or subjects classified as control subjects (CNTRL). As can be seen from the boxplots and Tables 7 and 8, individual markers do not achieve as high a level of sensitivity and specificity as Combinations 1 and 2 in classifying and detecting the identified colorectal neoplasms.

### Other Embodiments

While we have described a number of embodiments, it is apparent that our basic disclosure and examples may provide other embodiments that utilize or are encompassed by the compositions and methods described herein. Therefore, it will be appreciated that the scope of is to be defined by that which may be understood from the disclosure and the appended claims rather than by the specific embodiments that have been represented by way of example.

All references cited herein are hereby incorporated by reference.

SEQUENCES
uid: 1_34165443_34165675
(SEQ ID NO: 1)
GCCACCTCCACCTCCAGATAAACCACAAATTACAT
CTAAAGGGTTGTTTATCCGTGTCTGTTTTGCAATT
GACCAGTTTCTTTTAAGTTCAGTCCTCCTGTTTTC
ATTTATAACATCACCCATTAATACACCCCCCTCTC
CACACACACACACACAAACACACACACACACACAC
AGTGACAGAGACACACGCACTCACACACACAGGCA
CATACACGCACACCTCTTCCACG
uid: 1_219928356_219928386
(SEQ ID NO: 2)
CGGAGACCAGCTCCGCCACGAAGAAGGCGAC
uid: 1_161539915_161540181
(SEQ ID NO: 3)
GTCGTACTTCTCTGACCAAAATCAACGGAACCCCC
GAACCCACAGAGAGCATCCATTTTGGATTCCCCGA
AGACCCTTCGTTCCTGCATTCTCTTCTGCTCTCCT
TTTCTCAACCTCTTCTGCTGGAAGCTGAGTCCTGC
TCCAGATCTAGGCAAGTGCTAGCGCAGAAAAAAGA
CCTGCCTCGCTCAGGGCTATGAGCCGCGCCCTGAA
GCACGGAAAGCTAATTGTGTCACTGGTTTCAAATC
AACCTCAATTTTTTTGGAGACG
uid: 1_150294178_150294265
(SEQ ID NO: 4)
GATTCACGTCTACTTTCTAGGATGACTTCCATGTG
CTCCATCTCGCGCGTCCCTGAGCATGTTGAATTTC
CAAATCCTAAATAAGCCG
uid: 2_47569627_47569858
(SEQ ID NO: 5)
CGGTAGCCCTTGGCACGTATTCTTAGAGGAGAAAA
CGGAGGCTCACAAAGGTCAGATCACAGAGCCGGCC
AGTGTTGGAGCACAGGCGGCCCGGGGTGAGCGCCA
GAGGTGGGCTTTCTTCCCTCACTGAAAGCCGGGAG
GGAGAGAGAGAGAGAGAACGGGGGCCGGCGGAGAA
GAGGGCGAGACGAAAGTAAGCAAAGGGACATTAGA
AGGGAAGGCAGAGCCGAGGGAC
uid: 3_157437029_157437296
(SEQ ID NO: 6)
CGAAATAGACAATGGACTCCATCCCACTGAGGACC
GTAAGTTCACTTTAACTGTTTCTCTGCTAACCCTG
ACTACATATCCACCTCTTGGTCTAAATAACACACA
TATACTTTGTGGCCAGTGAGACAAGTTAAAAATTT
ATAGCTTGTTATGCAAAAGTGAGAAGCACTTGAAG
AAAGATGGAGGTTTCAAAGTTATTTCTGTAACGTA
CATAATGGGTTGAATCATATCAAATCGTCAATATT
TGACTGTTTTGAGCTACATAACC
uid: 3_173397284_173397387
(SEQ ID NO: 7)
CGTGTCTAAATAAGTACATGACACTAAATTTCCTT
TTAAATCCACCTTTTACACCATGGCCAGTCGCTTG
TTTAACTCCCGTTCAAGGGACACGGTTTTCAAAC
uid: 3_43998153_43998214
(SEQ ID NO: 8)
CGCCCAGATTCACGGTCCCCACGTGTTGCTGGAGT
TGGCGCAGACGCGTGTGCGGGCATAGC
uid: 3_10993689_10993900
(SEQ ID NO: 9)
GGTAGCCTCGGGCAGTGCCCATTGGGTTCTGAGCA
CACGTCCCCACGGGTGGCACCCACAGATGTCCTGT
TCTAGGCTTGGCTCGGTCTTCAGACAAGAAACTCA
GACCGGGCAGTCCCCTATTGAGGCTCTGAGCTAAT
ATCCTCCCAAAATAGACATGAACCACAAGGAGAAT
TTTTTAAAAGCCAAATGATAACACCACGTTCTTTC
CG
uid: 3_181712580_181712614
(SEQ ID NO: 10)
CGCCTGGGCGCCGAGTGGAAACTTTTGTCGGAGAC
uid: 3_180679674_l80679706
(SEQ ID NO: 11)
CGTAACTTTCCACGAAAAGGCGGCTCTCGGATC
uid: 7_65252405_65252749
(SEQ ID NO: 12)
GCTGTGCGGGTCCGGGACTCAGGGTTCCCGGCTGC
TATCAAGGCTGCGTAGCTTCCCCCTCCCCTCCTCC
TTAGGTGGCAACTTGTGGACACACCCATTAAGCGG
TCAGGCGTCAGGTTTCCTCCCGAGAGGTGGGAGGC
GCCCTGGCCTTGATTCATCGTGAAGCTAGGCAGGA
GATTTCCCAGCCACGGAGGGTGGAAAGCTTGCCTT
GACCTCAGCAGGTCATGTCACTCCGTGTGCACAAG
GCCTCGAGAGGCACTTTTTAAAAATTTTTTGAGGC
GGTTTTCTTTTTTGTCTTTGTCTTTTCTTTTTTTT
CTGAGACAGAGACAGAGTCTCGCTCTGTCG
uid: 7_64314242_643 14346
(SEQ ID NO: 13)
GCGGTCTGCTCTGGAGTCTGCATCCCCGAGTCCCC
GCGGGCACAGCTCAATCCTCATTTCCCTCCATCGC
AGATTAGGGGCTGAGCCAGCAGCCAAAACGCTACG
uid: 8_53877760_53878892
(SEQ ID NO: 14)
CGAGTTCAACTCACCCAGGAGCAAACAAACGACAG
CAAGACAAATCAGCCACCGCACTCGCGGCTTCCCA
GAAAGGGCCTCATGAATGAGAATGGGTTGCTAGGT
TTCCTTCCCTCTCTCCTGACAATCGCTTCCCACAA
GACTTCCACCGCCGAAAGAATACAGGCCGGGCCTG
GTGACTGCGGAGTGAGGGAACCGCGCCAGGCCCAC
GAGGCCGCTCGCGACCGCTCCCGCCTTCAGGACCC
TGGAGAGCGGCCGCCGCGCCCCTGGGACCCACGCC
CAACCCAGAACACCCGCGCTCGCCTCCCGCGCCTC
ACCACCCCAGCACTTTATTCGCTGCTTCCCGCCTC
CACCTTCATTTTTTTTGGCAACCACCGCTCTTGTT
TTTCACAGACAGATTAAATTGTTTTTTGTTTGGCA
TGAGGGGGTGTTTGGATTGGCCGAGCTACATTCCG
GTTTGTAACTACACTAAACGTTAGTGTACCAAGAA
ACTAAGAGCGTCTCAAAGATGACAGTCTGAACGTG
GCAGGTGACCTTAAAAAGCCACTGAGTTCAACCCC
TTTATAAATGAACTCAAAAAATAAAGAGACATTCC
CAAGGTTAGCCATTAAGTTAGTGGCAAGGTTGAGT
GTAGAACCGTGGGTTTCTGACTTCTAAACTAGTGC
TTCCCTCCACTTTCCAAAAGAAAAAAAATATCTCA
TTGCTTAGGTCTCCTAAGCAACAAGTGGAAGGTGG
AAAGGCCGGTGTTCCCGGGAATAAGAAGGACACAT
GGAGTTTGGAAAGGAGGTCTGCAGAGGGTTCACTT
GTAGTAGCAGCCCTAACATGGAATCCCGGTGTCCC
CAGCTCCACGCAACATACCTACAGCCGCACTGGCC
AGTCCCTTTTTCCAACCAGCCTGTGGAGGGTCTGA
GGCCCTTCAGCCACCTTCAGCCACTATCTGGACCT
TCCATAGACAACAATCCCAATAGAATCTAGTAGCC
AAGGCCAACTCTGTGCCTCTGGCAGGCAGAGCCCT
GGGCACTTAGGGAGATGGGAAAGGGCAGGGGGAAC
AAGGGAAGACCAGAGAAGGGAGGGGAAGAGCAACA
GTTACAAAAGTGAACTAAGATTTTCATCCAAATTC
TGAAGAACCAGGC
uid: 8_66177597_66 177765
(SEQ ID NO: 15)
GGGACTGCCTTAGACGTGCTGGGCTTTGGCCTCAG
TGATTCGGATGGGCGCTGTCCGCGGTGCTGAAGCG
CCTGGGGAGCGGGGAAAGGGGGCGCCTGCAGGGCT
CCGCACCTAAGCTCATGCGCTCACCGGCCAGGAGT
GACCCTTCTTTCTAGACTCTGAAAGGACG
uid: 8_108787618_108787717
(SEQ ID NO: 16)
GGCGGGCGGGGTGGGGCGGGGCGGGGCGCTCCCGG
AGCATCCCGGGAGTTGTAGGCCAGGGGCGGTCCCT
GCATCCCTCCTGTCTCTGTCTCCTCCCACG
uid: 8_53881543_53881774
(SEQ ID NO: 17)
CGCTGGGAAGGGTAAGGAGACAGAGCCTCCTGGAA
TCGTAGCGCCTCCTTTTAGGAGAAGTGCAACCAGG
GCAGGGGCACCGAGGGGCAGGGTGAGGAAGTGGAC
GCCCCACGCGTGGACCCTAGAAGACCGACTAGGTA
TGGGCGTTCACTCGGAGCCTCTGTTCACGCTCTTG
AAAACCGAATGAACAGAGAAGTGCCCTCCACCCTT
CGCCTGCGCCAGGGCAGTTAAC
uid: 4_183905237_183905728
(SEQ ID NO: 18)
CGGCGCATTGGCCCCTCCTCCCCTCGCGCGCCGCG
CGCATTGTTGTCCTTTAGCGATTGGTTGTTGGACC
AGAAACAGCTGTGCAGAGCCGTGCCATCTAAAGAG
CTGTGGACCTGAATGCAGCGTAGCGGGCTGGCGGT
GACTTACACCGGGACTCCAGAGGGAGAGAGGAAGC
GCTGCAGGCCACTTGCATTGCGTCTTCCAGGCTGC
GTGGACCCGGCGCCCCGGCGTGTGCGGTTGTGGGG
GAGCTCGCCGTGGCCTCCCCTCCCTCTGGCTTTAG
CTTCCTTTGGGGTTGGCGCAGGTGGGCCAGGCAGC
GCACCGCAGATCTCCCCGTTCCCACGAAGGCTGGC
TCGCTGTCTCTCTCCGAGCGGGAGGGACCATCCTA
AAAATATGTAAATATCCAAGCGCTGGCTCCAGGCT
GGGGCAGCTGCCAAGGTCCCCGCGCCGCCGCCGGG
TGTTTTACATGAAAATGAGAAGCCTGATGGGAACC
GC
uid: 4_183904880_183905140
(SEQ ID NO: 19)
CGATATCAGCCTGTTAGAAGCATATCCCTATAAAG
ATTTAATATCCCTGTCTCTGCATCTTGGCACCTGT
GAATATGAAACAACAGCATAAATATGATTTTGAAC
GTTGCATTGTCACAGATGAAAAAATGCACCAACAT
GTCAAATGCAGCGCTGAAAAAGGAAATCGGGCTTA
TTTTTGTCGTTGTTTACTGTACCAAAGCATTTTTG
AAAACCCAAATCGAGGAGATAACCGTTTTTGAATG
AACGGCAGTGCAAAGC
uid: 4_13545274_13545403
(SEQ ID NO: 20)
GACCCAAACCACGTTTCTTACCTCTGCAGATGTAT
CCACTTATTCCAGCGCTTTAACAGAACACTGATAC
TAAGTTGAGTCAAATCTGCGGAGAAAATCCAAGAT
AATGCAGTTCCATAAATCATGCTCG
uid: 4_127623100_127623235
(SEQ ID NO: 21)
CGAGCCCTGGTGGGGCTGGCGGCCCACAGAGCCCC
CACCTGCCCCGAGCTCCCACAGCGAGGAGTGGCCG
CGCCGCCCGCCAGTGCGCCGGGCTCCGAGACCGGC
AGGGGAGCACGCGGGCGAAGGAGGGGCCGCC
uid: 4_30718010_30718076
(SEQ ID NO: 22)
CGTTCCGATAAACCCGGGTTCTTGCCAAATGTAAG
AGGGATTTGGCTTTACTGCCACTTAGCCGGCC
uid: 4_82562331_82562477
(SEQ ID NO: 23)
GTCCGCACACCTAGGGTTTTTGCACAAAGCAGCTA
AAATTCATTTTCATGCCCATTGACTGAATGTAGTT
CTCCTCCACTCAGCGCTGTCCCGCGGTTTTTGGTT
TTTTTTTTTCCATCCTATTGAACAAACACAGAGCC
AACTTCG
uid: 5_88660609_88660834
(SEQ ID NO: 24)
CGTGCCGCCTGAGGAAGGTGCCCTGTGGCAGGGGG
TGGCCGCTGGGAGATGCCTGCCTCTAACCGACGTC
CAGGCGTGACTAAACCTCGACGCCACCCCCATTCA
CAAGCTCAACTCAGGGATTCCCAAGCAACCGCAGA
CCGGAGGTGGCGCAGAGGCAGTGACCGAGGTCGCT
GATTAGGGGCCGAGAGGCTGGCAAATAATAATTTT
AAAAAAAAAAAAAACC
uid: 5_83474370_83474485
(SEQ ID NO: 25)
CGAGACTCGTTTTAGGATACTCTTTTCCCTTTCCC
AGCGGCAACAAATGTGACTGCAGAGGCGTGCCGGG
ATGGAAGGGTGGGAAAGAACTAGACAAGCGGAGGT
GGTCCAGCCCC
uid: 5_79512985_79513164
(SEQ ID NO: 26)
CGGCATTTGCTTATTCGAGTTAAAATGCTTTGCGG
AGGAGACAGCGATCAACTCTATTCCACAAAATGAG
TCTACAAGTAGGGAAATGCAGAATCCGGCTTCACC
GAGTCCTATAAAAAATGAGTTCGCTGGTCATTTCA
CTCATGTCCTCCTCGACACTCAGGGAGAGCCAGGT
AACTC
uid: 5_51387638_51388080
(SEQ ID NO: 27)
CGGGAGGACGGTCTCTTCTGCCGAGCAGACCACGA
TGTGGTGGAGAGGGCCAGTCTAGGCGCTGGCGACC
CGCTCAGTCCCCTGCATCCAGCGCGGCCACTGCAA
ATGGCAGGTACTCCTCTGCCCGGCTCGGGTAGGCA
GGCGCCAGGTTAAGCCAGCCTGTGTGCCAGCGGCC
ACAACAACTATGGTAGCTACAGGGGTGGTCGTAGT
GTTTGCCTGCAGTTAAATGAAGTGTTCTGTATGCA
ATTTGCGCTGTGCTCTGCTCCTTTGCAGCAAGGTT
CAATGCACTCACTGTCTCCCTTGATTCCCCGAGCA
CACCTACACCGTCTGTGTGTCTCTATATGGTTACA
CATAAATGTACACCACTTGTGTACACGTGTATACA
CACGCCCAAACATTACTTCCAGTTCGCTCTGGCCT
CCAAACCTTGGCTTGCTGAAAAC
uid: 6_129767354_129767459
(SEQ ID NO: 28)
CGGAAGGACCGGGCTGAAGCGTGGCCACGAGGAGG
GGGATACCCGTGCGAGCGCTGAGCCGGCAGAGCGG
CTGCAGCCCACGGGCTCCTCGGACCCCCGCTGCTG
C
uid: 6_27288050_27288544
(SEQ ID NO: 29)
CGTGGAGGGGGTGGGGTGGAAAGAAGTAGGGAATG
GAGAAGATTACTAAGAAAAGTTTCCTGTCTGGAAC
TGCGGCAGATCTCTTTGGATAGAGATGACTACTTA
ACCTCACTCTGCTTTCCTTCGCCGCGGTTGCGGCC
GCGACCCTGTTCTTACCACCAGCAATTCCTCCAGG
GACTTGGTCAGCAGCCCAACTTGATCTGCGTCTCT
CTGCTAAGGTGTTTCCGCAACAGGGTCAACTCCAA
GTCTCACCTTTCTAGGAATCCCGGGCGCAGCGCGG
GGGTCGGGACTCCGACCTGTATTTCCAGGCGGAGG
TTTCCCTGGGTCAGGCGGCCACTCTCTGCCAGAGA
TTGTCAGTTATCCAACTGTCAATAGAGCCGCCGCT
CCAGCGAGTTTAATTTAGGCACAGAAAAGTCCTGC
CTGGGTTGAGGTGGGCTTAGGATGAGTTTACTTGA
GTGTGTGATTTAGAAATAGATCTATGGGACAGACA
GACAC
uid: 6_165309346_165309582
(SEQ ID NO: 30)
GCACCCCCCACCTCTGTGCTTTGCGCGCTGGTTTC
AGATTCTCTGAGGAGGCAGCACAACCCCAATCCAG
TCCTCAGCCCTAGTGACCCTGGAGCCGGCGTGCAG
CCACCAGAAAAAGATTTTCACATTTCAAATCAGTC
TTCAGAAGCCCATCCCTCCACAATTGAACATCAAG
CAAATCTCACAAGCCAGCAAGAGCCGCTCCAGGCC
ACTTACATTCAGGCTCCCGCTCCTCCG
uid: 6_6320205_6320663
(SEQ ID NO: 31)
GGGGTTCCAGTCTAGCAGGCTGTTCTCACTTGGCC
CCACTCCCTCCACCTTTTGTGTTCCAGCTCCATAA
TCTGCTCCCTGAGGAAAGGGGGCTCGTCCCTTGGG
GAAGCACCTCCAACTCCCCCATCCCCATTTGGTGG
CATTCTAAGCAAGCAACGGCTTCGGGAGAGCTGCC
TCGAGAGCCTGAGAGAAGTCCCGCTTAGAAGCTGG
GCTGGGCAGGTGCGGAGTTGGGGGCGGGAAGCCAG
GATTGGGCAAGTGGAGCTGCCTGTGACCGGCGCCA
CAGGGCCCAGAGCAAGCCGCTTGCTGGTTCAACCA
GGAAACCGAGGTGCAGAAGGTGGACGCAGCGGGCC
CTGGCTCATAGGGTGCAGGGTCGGTGGCTTACCTG
CAGGCGCTCCCCTCCAGAGGTGCCCTCGCGTGGGC
TTGCTCTGTGCGCCTCGGGGACTTCCTCAAACGGA
CTCG
uid: 6_68635305_68635560
(SEQ ID NO: 32)
GAGACAGAGCTTTACTATCTCGCTCCCTCTCGCGC
CTCCCTCCTCGCTGGGCATTCAAACAGCTTTCCGA
CATCACCAGCCAAGGATTTTTTTCCCCGCTCTCCT
TAGTCGCCGTCCGTCCATCAGTACCTGCAGGGGGG
AGGAGGAGGAGGGAGGAAAGCGGAAAGAGGAAAAA
GCATAAGCTTGAGCCTTCCGATCCGACCACGAATA
CTCCTGTAATAAACCCACCGCCCCAACAAATCTGC
CATAGCAGCCG
uid: 6_27670532_27670614
(SEQ ID NO: 33)
CGGCTTTGAAGAAGGAAAAAGTGAGAGCACAAGCG
AGCCAGCCAGGAGTCGAACCTAGAATCTTCTGATC
CGTAGTCAGACGC
uid: 6_1524106_1524152
(SEQ ID NO: 34)
CGCGACGGCCCCAATTCCAGCAACGCTAGAGGGCG
CCCGTGCCAAGC
uid: 11_79438095_79438308
(SEQ ID NO: 35)
CGCTGGAATGTGGCAAAAACAAAACACTCCCCACC
CATGCACATCACCATCTCCTGACTGCGGGCTGGGG
GGAAGGGAGTTCAGGGCCAATGTGTCCCAGACTTC
AGCGTTCCCCACGGCTGTGTCAGGGCTGGGGTGGC
TTATCCCCCTACAGACGAAAATCAAGATTTAAAAG
CATACTCTTACTGTGGTTTCTCTATCAAAGCCCAT
CAAC
uid: 11_94740046_94740862
(SEQ ID NO: 36)
GCATAAATTAACAACTTCTCAGCCTCGGTTTCCCA
ACCTGTGCAATGCAGCTCATATTACTTTGCCCACT
TGAGCCAGGTGATCTCTTGAAGAGTAGGAAATAAT
GTTGGACTGGAGTTGGAATTCTGGTTCCAAAAGGA
GGGGATTGGGGGGCATTTGGGTCTCCTCCTACCTT
TCTGCAAGCTCTGAAAATCGTCCATCTCCTCAGGA
GAAGCCCACAGAACAAACGATTTCCCAAGCATCAG
AGGAGGGCAGACATAACATAACAGAGCAGGGAATC
GGGTTAGAACGGGTTATGCTCTGATTTTTTGGAAA
ATGGGCAAACGGGGCGGGGACTAGGGAGGATTCCG
CCAGCCGGGAGTTGGGAGGCCGCGCGCGCCTCTGC
GGAGCGTGACGGCCACGGTCGCCTAGCAACGAGCG
GGGATGCGCGGGGCGCCTGGGTGGGCGAGGGGTGC
TCGCCGCCGCCGCCGCGCGCCCTCGGGCCCCCGGG
ACCGCGGGAAAACTTCGCGGCTCCTCGGCGCTGGG
GCGTCGGCGCCAGGCCCGGCAGACAGAGCAATGCG
CCCCGCGGGCTGAGGGCAGAGGATGCGGCCGCGGC
CCAGCGCCCGGCCGGGGGAGCCGCGGGGTGGCACG
CGGGGAAAGTTGGCGCGCGCCTGACCGCGCCTGGA
AGCCGCGCGGTGCCAGGGCCGAGTTGTCCCCCAAG
TTTCTGCGGCGATTTGTCACTCCCTGGGGATCTGG
CGGTCTGAATCCCGCGGGGCCTCCGGCTCAGGGAT
TCTGAGCGCTGGGAGAGAGAAGCCCGCGCTTTTCC
CGGGGACCTGCG
uid: 13_93228702_93229444
(SEQ ID NO: 37)
GGGCTGCTCAAACCGGGCAGCTGAAGTCCTTAGTG
ACCTCAGATCGTCAAGTCAAAACGCTGAATTTCCA
CCAGCCTCTGTCTGCTTTTTGCCAAATAACTGGTG
GATGGATGAAAAGCATTTTGCAGATATCTTAGAAC
ATCACAGTTTCGATACGTTGAGGAATTACTATTTT
CTTATGATTTTCAAGCTGTAGAAGTGAGGGTTTTT
ACTTACACTGAAATGAACACATTTAAATAAATTTG
AGCATTGGCAAAGGGGGAAAAAAAGAGGCGCAAAT
TACCACGCTCATTATATAGAAGGAGCTTTTTCAGT
TCAGAGCCAGACATTCCCTTTGCTGAGTCTAAGTT
AGAATCTGTGGTGAATTATAAGCCTACTTTTCTAT
CCTTGTTACTTCTTCCTTCTTTTCCAGAACTCCTT
AATTTGTTAATCAATGAATAGAGAGCGACTGTCCC
CACAGCTCCTTAAGTTTCTTAACTCTCCTTCTCCT
TTGTCTACTGTTATTTCATTCTTTTTAATTAATTG
ATAAGGATCAGCTTCGCTTTTTTTTTTCCCTCCCC
AAATCTCAGGGAATTCAACTTTTTAAAAGGTTTAT
AGTATGGATCACTTCTTCTAGGAACTTTTTCTCCT
TTAATCTGGGCTTTTTCAAACGGTATCTTTTAAGC
ACACAAGATATTTCCCAACATGATTTCAGATAAGA
TGTCTCAAAGAAGAAAATAGTTAGGTATTTTTAAA
TTGCTTCG
uid: 13_94712995_94713032
(SEQ ID NO: 38)
GCCTGCCGACTTAGGGCTCCCGGAGCTCGCCGGCC
GCG
uid: 13_67231493_67231965
(SEQ ID NO: 39)
GTTTTCATACGGGAACTGGATGGAATGACTCCCCA
AAAAATACAGCTTTATTTCTCAAATACTGACCCCC
AAAGCACTATCTAGTAATATATTTGATTGATCTTT
CAAAGTCAGTAAACCACAAAGGTTTGTGTAATGGC
TTGTACTTAACGCCTGATACCTGAGTAAAGTTTGA
AGCATTAACATAGGAACAGTTCACTTGGAACAAAA
ATTTATTTTCTGAATGACCTATAAAGGTTGTCAGA
GAAGGTCTTAGTACATGATTGAATAACTTGGTCTA
ACTTACAGGAAGAATAAAGGGCATTTATTTTAAAC
CTGTGTGTTCCCCATTCTATAATGGGCTGCCTAGG
CATTAAAGGCTCTTGACATACTTAAGCTCTTGACT
GAGGGCGTGTTGGAGATTACCCCTTGTTTTTGAGT
ACAATAGTTTTGTTTGCTTTTTTTCTTTTTTAAGT
AACATTTGTTTTTAGACG
uid: 13_61414743_61414845
(SEQ ID NO: 40)
CGATCCCGAACCCCTGTAGCTAAAGCGGATTGAGC
GCACCCCCGATGCCCTCGACCTTATCTGGATACAT
TTCTTGCTTCAGAAACTTCCTCTCATGACCCAC
uid: 9_132579614_132579683
(SEQ ID NO: 41)
CGAAATCGGAATAAAACGATGTTATTGAGAGAGGG
CTAAATCCCAGAGTAAATTTCAAACGAAATAAATC
uid: 10_22475853_22476043
(SEQ ID NO: 42)
CGCGGGGTGCAGCATGAGTCTTCCTTTGTGGCGTG
CGGCTCCATCGGAACGCGCGTTGCGACGACAAATT
CCTTTTTTCCCCCCCGCAGTTAACAGTTCTGGGGC
AGAGGCTGGTGGAGAGGTCCAGAGCCCACTCAGAC
CGAGATGAAGATGAGGAAAAGCATGAGCAGGAAGA
GGCTGGCGGCTGCGGC
uid: 10_117165171_117165431
(SEQ ID NO: 43)
CGGAGCCGAGTGCTCGGTCTCTTTCCCCGGGACGG
GACAGGGAGTGGAGTTCCTAGCCCCTTGGGTGGGA
AAAGCCCCGCGCACGTCACCGGGTCCCGTCTGCTC
ACTGCTTCTGCATATTTTAAGCCTGGACACAGCCT
CCTTTAGGATAGAAAGGCATTTCCCAAACAACACC
GATTCTGGGGGTGTAGTGGGCCTGGCGCTGGGTCC
TGGAGAGAAGGTTCAGCCCCCCTTCTCATCCCTGT
ACTTTGGGGATGGCTC
uid: 10_16521408_16521447
(SEQ ID NO: 44)
GGCGCCGGCGCGGCCACCGGGAGGGCAGAGCCAG
CCGCCG
uid: 12_78865146_78865306
(SEQ ID NO: 45)
GGTGCCTATCTGCTCGAATCCATCCCAAAGATTTT
CTTCTCGATACACTTGGGTTTTAGTGGTGGGAGTC
TGAGACCTAGGGAGGGATCCCCGGGTGGCCTATTG
TGAGAAATATGAGACTACATGTGCATCTCCTGGAA
AAGCACTTTGCACAGCGCCCG
uid: 12_15221303_15221461
(SEQ ID NO: 46)
GTTTGCGAGTTGCTGGGCTGCGGTCGTGGGTGGGA
CTCGCCGCAGAAGCAAGTGCCAGTGGCCCGGCGGG
GGTCTCCTCACTCGCGCTCGCTCCGACTAGCGGCG
GAGGGACTGCGGCAGGACGCGAGCTGAGCCCGGCC
AAGGCCGCTGCGCTCAGCG
uid: 12_24563707_24564068
(SEQ ID NO: 47)
GTCGGTGATGCCCTGAGCAGAAGCTGTGGGGCTGA
TTTAAGTGTGTGCTCTGACAGCTCCCGGGCTGCTC
CGGCGCTTATCTCTTCTAATCTTACTTTCATTGAC
TTAAAACAGCTGCCGGTTTGCCAGGGGAAAAAAAT
CCTATTAAATCTCAAACCAGGGTGGGGCGGGGGCG
GGGGTTCCTGACAGTGATCTTCCCAGAGCTAGTGT
CCAGAAAAGAGCAGGGGGAAGGGAGAGCATTCACG
GGGGTGGCTGCGTGTTGGGAAGGGGGTGATGGAAA
GGGGACTAAGGAAACAATTCGAGCAACACGTTTCT
TTTCCTCGCTGTAGCCTTCTCTCCTTTTGCTTTTA
TGCCGAGGTGCG
uid: 12_53974529_53974571
(SEQ ID NO: 48)
CGGCGACAGGGGAATGGGGCGAGGCGGCGCAGGACT
CCACTGC
uid: 14_59870236_59870262
(SEQ ID NO: 49)
CGAATATTCCCAGTCGCCCGTGGCGAC
uid: 18_27184277_27184863
(SEQ ID NO: 50)
CGCAGAGAGAGAGAAAGGAGGAAGGCCTGCAGCTC
TAGACTTAGCACCTGTGAACTTAGTTGGAAATAAC
TCCTGACAGACATCAACCATAACACCTTCTAACAG
AAACATGCTGATAGGCAGGATGTTCAAACGGGGCA
GAAGAGGGAGCAGGCAACCCTGTGAAAGTAACAGC
AGCAGAAACAAACACACCATTCCTTCTCCCCGCAA
CCCACTTGGAGCCCCTCCTCAGATCGCCCCTCCCA
ACCCCTCTGCTCTGGCCACGATCGCACCTGGCCTC
CCGGTCCCCCTAACTTCCCTCCCACCTCTCCCGCA
GCCTGCGCCTGAGCCTGAGCCAGGTCGCGGAGTTT
GAGACTGACGCAAAGGAGGCACCCCCGCAGCAGAG
ATGCTCGTCTTTCTGCCACACACCCTGGAGGACCC
GACAGACTGGCAGCAGAAACTAAAGACTGTTCCTG
CCGTCCTCTTTCCAACCTCTGCATGCCCCAAGAGG
GGTCTGACCCCGGAATCCTGGAGTCCAGGGTGCCC
CGCCGGGGCGCAGGAAGGAAACTCAGGTAGCTGAC
AAGTTCAGGCGGCCGTCCTTCTCCAGC
uid: 20_9516555_9516632
(SEQ ID NO: 51)
GCCAGGAACCGCAGGCGTGGGGACCCAAACGTCAC
CCGTGGCCTGATCCTAGATAAGAAGTCCCTTGAAG
GCCTGTCG
uid: 213134410431344160
(SEQ ID NO: 52)
GGTGGAAAGAATCGATTTCAAAATTCAAGCTCACC
GCTGCTCAACAAGGCGCGCACG
uid: 19_20052466_20053193
(SEQ ID NO: 53)
CGCCCCGTTGCCAGGGAGAGTGAATACAGCTAGGG
ACGTGAAGGGTAGGTCTGGGCTGGGCATTGAGGAG
GGTATTAGGCTAGGAAGTATACCTCACCTTTGTCC
AAATCGGGTGGTTCGGCCTCCTCTCATTGGCCCTA
TGCAGCTGGGTTGCCTCTCTCACCCGCACCCAAGG
GTCTTTCCAGAGTGTTGCGTCATTTCCAGCCCAGG
GAGCTGCCTTCTTTCCTAAACTGCAATGGAAACTG
TCCTGATGTCTGAGACAATGTCCGTTGTGCCGCAG
CCCTCTGCCTTCTCTAGCCAGAGCGCGAGCTCAGC
TGCTTTTGAGAGAAATCAGCCACCTGGCCCACCTG
TGCACAACTTCAGAGCTTTGTAGGGGGTGACAAGG
ACTGTGTCTTCAGGGAAATGTCACGGGCATTAGCG
CCTTTTTCGCAAATGTGAAAGTTGAGAAATCAAGA
AGGTTAATTATTGGGTTGCCCAAGATCTGCTAAGA
GCAAAGGAGAAAACTCCGTTTCCCAGGCATGTGTC
TTGTGAGCCATTTTTAAATCAACCCTCTTAAGTGG
ACAAGCTCCAGAACACAACATGAAGCTGATGATGA
CTTAGGCAATTTATGCTTGAACTCATTGGCCTCAT
CTCAAGTCAGTGTCTCAGAGACACAGGTGGGACCT
GATCCCCAAGGAACAGATAGCATTCCAGATTCATG
GGAGCAACTTTTGAGATGTGGAGCACCC
uid: X_625391_625465
(SEQ ID NO: 54)
GGTCACATACGCTAACAAGACACGGTGAAAAGTCT
CTTCTCATCGGCTTGGTGTGCTGCTCTCTTCTCTC
TCTCG

1.-10. (canceled)

11. A method of detecting a methylation status in a human subject suspected of having a colorectal neoplasm, the method comprising:
determining a methylation status for a methylation locus within gene BARHL1 in a sample of deoxyribonucleic acid (DNA) from a human subject suspected of having the colorectal neoplasm
and
detecting the colorectal neoplasm in the human subject based at least on said determined methylation status.

12. The method of claim 11, wherein detecting the colorectal neoplasm comprises a member selected from the group consisting of (i) classifying the subject as having advanced adenoma, (ii) classifying the subject as having polyposis, (iii) classifying the subject as having colorectal cancer, (iv) classifying the subject as having at least one of the conditions advanced adenoma, polyposis, and colorectal cancer, either with or without identifying which of those conditions the subject has, and (v) classifying the subject as having at least one of the conditions advanced adenoma and colorectal cancer, either with or without identifying which of those conditions the subject has.

13.-14. (canceled)

15. The method of claim 11, wherein the methylation locus within gene BARHL1 comprises at least a portion of BARHL1 '614 [chr9:132579614-132579683] (SEQ ID NO: 41).

16. (canceled)

17. The method of claim 11, wherein the DNA is isolated from blood or plasma of the human subject.

18. The method of claim 11, wherein the step of determining the methylation status further comprises determining a relative amount of methylated and unmethylated CpGs and/or determining a read-based pathological methylation pattern.

19. The method of claim 11, wherein methylation status is determined using methylation sensitive restriction enzyme quantitative polymerase chain reaction (MSRE-qPCR).

20.-21. (canceled)

22. The method of claim 11, wherein the methylation locus of BARHL1 is or comprises at least one CpG dinucleotide.

23.-44. (canceled)

45. The method of claim 11, wherein the DNA is cell-free DNA of the human subject.

46. The method of claim 11, wherein methylation status is determined using massively parallel sequencing.

47. The method of claim 11, wherein each methylation locus is equal to or less than 5000 bp in length.

48. The method of claim 11, comprising determining the methylation status of each of the one or more markers using next generation sequencing (NGS).

49. The method of claim 11, comprising determining whether the methylation locus within gene BARHL1 is hypermethylated and detecting the colorectal neoplasm based on, at least, hypermethylation of BARHL1.


SEQUENCE LISTING (54 sequences)

SEQ ID NO: 1 - DNA, 233 bp, synthetic construct
  source 1..233: mol_type = other DNA; note = Description of Artificial Sequence: Synthetic polynucleotide
gccacctccacctccagataaaccacaaattacatctaaagggttgtttatccgtgtctgttttgcaattgaccagtttcttttaagttcagtcctcctgttttcatttataacatcacccattaatacacccccctctccacacacacacacacaaacacacacacacacacacagtgacagagacacacgcactcacacacacaggcacatacacgcacacctcttccacg

SEQ ID NO: 2 - DNA, 31 bp, synthetic construct
  source 1..31: mol_type = other DNA; note = Description of Artificial Sequence: Synthetic oligonucleotide
cggagaccagctccgccacgaagaaggcgac

SEQ ID NO: 3 - DNA, 267 bp, synthetic construct
  source 1..267: mol_type = other DNA; note = Description of Artificial Sequence: Synthetic polynucleotide
gtcgtacttctctgaccaaaatcaacggaacccccgaacccacagagagcatccattttggattccccgaagacccttcgttcctgcattctcttctgctctccttttctcaacctcttctgctggaagctgagtcctgctccagatctaggcaagtgctagcgcagaaaaaagacctgcctcgctcagggctatgagccgcgccctgaagcacggaaagctaattgtgtcactggtttcaaatcaacctcaatttttttggagacg

SEQ ID NO: 4 - DNA, 88 bp, synthetic construct
  source 1..88: mol_type = other DNA; note = Description of Artificial Sequence: Synthetic oligonucleotide
gattcacgtctactttctaggatgacttccatgtgctccatctcgcgcgtccctgagcatgttgaatttccaaatcctaaataagccg

SEQ ID NO: 5 - DNA, 232 bp, synthetic construct
  source 1..232: mol_type = other DNA; note = Description of Artificial Sequence: Synthetic polynucleotide
cggtagcccttggcacgtattcttagaggagaaaacggaggctcacaaaggtcagatcacagagccggccagtgttggagcacaggcggcccggggtgagcgccagaggtgggctttcttccctcactgaaagccgggagggagagagagagagagaacgggggccggcggagaagagggcgagacgaaagtaagcaaagggacattagaagggaaggcagagccgagggac

SEQ ID NO: 6 - DNA, 268 bp, synthetic construct
  source 1..268: mol_type = other DNA; note = Description of Artificial Sequence: Synthetic polynucleotide
cgaaatagacaatggactccatcccactgaggaccgtaagttcactttaactgtttctctgctaaccctgactacatatccacctcttggtctaaataacacacatatactttgtggccagtgagacaagttaaaaatttatagcttgttatgcaaaagtgagaagcacttgaagaaagatggaggtttcaaagttatttctgtaacgtacataatgggttgaatcatatcaaatcgtcaatatttgactgttttgagctacataacc

SEQ ID NO: 7 - DNA, 104 bp, synthetic construct
  source 1..104: mol_type = other DNA; note = Description of Artificial Sequence: Synthetic polynucleotide
cgtgtctaaataagtacatgacactaaatttccttttaaatccaccttttacaccatggccagtcgcttgtttaactcccgttcaagggacacggttttcaaac

SEQ ID NO: 8 - DNA, 62 bp, synthetic construct
  source 1..62: mol_type = other DNA; note = Description of Artificial Sequence: Synthetic oligonucleotide
cgcccagattcacggtccccacgtgttgctggagttggcgcagacgcgtgtgcgggcatagc

SEQ ID NO: 9 - DNA, 212 bp, synthetic construct
  source 1..212: mol_type = other DNA; note = Description of Artificial Sequence: Synthetic polynucleotide
ggtagcctcgggcagtgcccattgggttctgagcacacgtccccacgggtggcacccacagatgtcctgttctaggcttggctcggtcttcagacaagaaactcagaccgggcagtcccctattgaggctctgagctaatatcctcccaaaatagacatgaaccacaaggagaattttttaaaagccaaatgataacaccacgttctttccg

SEQ ID NO: 10 - DNA, 35 bp, synthetic construct
  source 1..35: mol_type = other DNA; note = Description of Artificial Sequence: Synthetic oligonucleotide
cgcctgggcgccgagtggaaacttttgtcggagac

SEQ ID NO: 11 - DNA, 33 bp, synthetic construct
  source 1..33: mol_type = other DNA; note = Description of Artificial Sequence: Synthetic oligonucleotide
cgtaactttccacgaaaaggcggctctcggatc

SEQ ID NO: 12 - DNA, 345 bp, synthetic construct
  source 1..345: mol_type = other DNA; note = Description of Artificial Sequence: Synthetic polynucleotide
gctgtgcgggtccgggactcagggttcccggctgctatcaaggctgcgtagcttccccctcccctcctccttaggtggcaacttgtggacacacccattaagcggtcaggcgtcaggtttcctcccgagaggtgggaggcgccctggccttgattcatcgtgaagctaggcaggagatttcccagccacggagggtggaaagcttgccttgacctcagcaggtcatgtcactccgtgtgcacaaggcctcgagaggcactttttaaaaattttttgaggcggttttcttttttgtctttgtcttttcttttttttctgagacagagacagagtctcgctctgtcg

SEQ ID NO: 13 - DNA, 105 bp, synthetic construct
  source 1..105: mol_type = other DNA; note = Description of Artificial Sequence: Synthetic polynucleotide
gcggtctgctctggagtctgcatccccgagtccccgcgggcacagctcaatcctcatttccctccatcgcagattaggggctgagccagcagccaaaacgctacg

SEQ ID NO: 14 - DNA, 1133 bp, synthetic construct
  source 1..1133: mol_type = other DNA; note = Description of Artificial Sequence: Synthetic polynucleotide
cgagttcaactcacccaggagcaaacaaacgacagcaagacaaatcagccaccgcactcgcggcttcccagaaagggcctcatgaatgagaatgggttgctaggtttccttccctctctcctgacaatcgcttcccacaagacttccaccgccgaaagaatacaggccgggcctggtgactgcggagtgagggaaccgcgccaggcccacgaggccgctcgcgaccgctcccgccttcaggaccctggagagcggccgccgcgcccctgggacccacgcccaacccagaacacccgcgctcgcctcccgcgcctcaccaccccagcactttattcgctgcttcccgcctccaccttcattttttttggcaaccaccgctcttgtttttcacagacagattaaattgttttttgtttggcatgagggggtgtttggattggccgagctacattccggtttgtaactacactaaacgttagtgtaccaagaaactaagagcgtctcaaagatgacagtctgaacgtggcaggtgaccttaaaaagccactgagttcaacccctttataaatgaactcaaaaaataaagagacattcccaaggttagccattaagttagtggcaaggttgagtgtagaaccgtgggtttctgacttctaaactagtgcttccctccactttccaaaagaaaaaaaatatctcattgcttaggtctcctaagcaacaagtggaaggtggaaaggccggtgttcccgggaataagaaggacacatggagtttggaaaggaggtctgcagagggttcacttgtagtagcagccctaacatggaatcccggtgtccccagctccacgcaacatacctacagccgcactggccagtccctttttccaaccagcctgtggagggtctgaggcccttcagccaccttcagccactatctggaccttccatagacaacaatcccaatagaatctagtagccaaggccaactctgtgcctctggcaggcagagccctgggcacttagggagatgggaaagggcagggggaacaagggaagaccagagaagggaggggaagagcaacagttacaaaagtgaactaagattttcatccaaattctgaagaaccaggc

SEQ ID NO: 15 - DNA, 169 bp, synthetic construct
  source 1..169: mol_type = other DNA; note = Description of Artificial Sequence: Synthetic polynucleotide
gggactgccttagacgtgctgggctttggcctcagtgattcggatgggcgctgtccgcggtgctgaagcgcctggggagcggggaaagggggcgcctgcagggctccgcacctaagctcatgcgctcaccggccaggagtgacccttctttctagactctgaaaggacg

SEQ ID NO: 16 - DNA, 100 bp, synthetic construct
  source 1..100: mol_type = other DNA; note = Description of Artificial Sequence: Synthetic polynucleotide
ggcgggcggggtggggcggggcggggcgctcccggagcatcccgggagttgtaggccaggggcggtccctgcatccctcctgtctctgtctcctcccacg

SEQ ID NO: 17 - DNA, 232 bp, synthetic construct
  source 1..232: mol_type = other DNA; note = Description of Artificial Sequence: Synthetic polynucleotide
cgctgggaagggtaaggagacagagcctcctggaatcgtagcgcctccttttaggagaagtgcaaccagggcaggggcaccgaggggcagggtgaggaagtggacgccccacgcgtggaccctagaagaccgactaggtatgggcgttcactcggagcctctgttcacgctcttgaaaaccgaatgaacagagaagtgccctccacccttcgcctgcgccagggcagttaac

SEQ ID NO: 18 - DNA, 492 bp, synthetic construct
  source 1..492: mol_type = other DNA; note = Description of Artificial Sequence: Synthetic polynucleotide
cggcgcattggcccctcctcccctcgcgcgccgcgcgcattgttgtcctttagcgattggttgttggaccagaaacagctgtgcagagccgtgccatctaaagagctgtggacctgaatgcagcgtagcgggctggcggtgacttacaccgggactccagagggagagaggaagcgctgcaggccacttgcattgcgtcttccaggctgcgtggacccggcgccccggcgtgtgcggttgtgggggagctcgccgtggcctcccctccctctggctttagcttcctttggggttggcgcaggtgggccaggcagcgcaccgcagatctccccgttcccacgaaggctggctcgctgtctctctccgagcgggagggaccatcctaaaaatatgtaaatatccaagcgctggctccaggctggggcagctgccaaggtccccgcgccgccgccgggtgttttacatgaaaatgagaagcctgatgggaaccgc

SEQ ID NO: 19 - DNA, 261 bp, synthetic construct
  source 1..261: mol_type = other DNA; note = Description of Artificial Sequence: Synthetic polynucleotide
cgatatcagcctgttagaagcatatccctataaagatttaatatccctgtctctgcatcttggcacctgtgaatatgaaacaacagcataaatatgattttgaacgttgcattgtcacagatgaaaaaatgcaccaacatgtcaaatgcagcgctgaaaaaggaaatcgggcttatttttgtcgttgtttactgtaccaaagcatttttgaaaacccaaatcgaggagataaccgtttttgaatgaacggcagtgcaaagc

SEQ ID NO: 20 - DNA, 130 bp, synthetic construct
  source 1..130: mol_type = other DNA; note = Description of Artificial Sequence: Synthetic polynucleotide
gacccaaaccacgtttcttacctctgcagatgtatccacttattccagcgctttaacagaacactgatactaagttgagtcaaatctgcggagaaaatccaagataatgcagttccataaatcatgctcg

SEQ ID NO: 21 - DNA, 136 bp, synthetic construct
  source 1..136: mol_type = other DNA; note = Description of Artificial Sequence: Synthetic polynucleotide
cgagccctggtggggctggcggcccacagagcccccacctgccccgagctcccacagcgaggagtggccgcgccgcccgccagtgcgccgggctccgagaccggcaggggagcacgcgggcgaaggaggggccgcc

SEQ ID NO: 22 - DNA, 67 bp, synthetic construct
  source 1..67: mol_type = other DNA; note = Description of Artificial Sequence: Synthetic oligonucleotide
cgttccgataaacccgggttcttgccaaatgtaagagggatttggctttactgccacttagccggcc

SEQ ID NO: 23 - DNA, 147 bp, synthetic construct
  source 1..147: mol_type = other DNA; note = Description of Artificial Sequence: Synthetic polynucleotide
gtccgcacacctagggtttttgcacaaagcagctaaaattcattttcatgcccattgactgaatgtagttctcctccactcagcgctgtcccgcggtttttggtttttttttttccatcctattgaacaaacacagagccaacttcg

SEQ ID NO: 24 - DNA, 226 bp, synthetic construct
  source 1..226: mol_type = other DNA; note = Description of Artificial Sequence: Synthetic polynucleotide
cgtgccgcctgaggaaggtgccctgtggcagggggtggccgctgggagatgcctgcctctaaccgacgtccaggcgtgactaaacctcgacgccacccccattcacaagctcaactcagggattcccaagcaaccgcagaccggaggtggcgcagaggcagtgaccgaggtcgctgattaggggccgagaggctggcaaataataattttaaaaaaaaaaaaaacc

SEQ ID NO: 25 - DNA, 116 bp, synthetic construct
  source 1..116: mol_type = other DNA; note = Description of Artificial Sequence: Synthetic polynucleotide
cgagactcgttttaggatactcttttccctttcccagcggcaacaaatgtgactgcagaggcgtgccgggatggaagggtgggaaagaactagacaagcggaggtggtccagcccc

SEQ ID NO: 26 - DNA, 180 bp, synthetic construct
  source 1..180: mol_type = other DNA; note = Description of Artificial Sequence: Synthetic polynucleotide
cggcatttgcttattcgagttaaaatgctttgcggaggagacagcgatcaactctattccacaaaatgagtctacaagtagggaaatgcagaatccggcttcaccgagtcctataaaaaatgagttcgctggtcatttcactcatgtcctcctcgacactcagggagagccaggtaactc

SEQ ID NO: 27 - DNA, 443 bp, synthetic construct
  source 1..443: mol_type = other DNA; note = Description of Artificial Sequence: Synthetic polynucleotide
cgggaggacggtctcttctgccgagcagaccacgatgtggtggagagggccagtctaggcgctggcgacccgctcagtcccctgcatccagcgcggccactgcaaatggcaggtactcctctgcccggctcgggtaggcaggcgccaggttaagccagcctgtgtgccagcggccacaacaactatggtagctacaggggtggtcgtagtgtttgcctgcagttaaatgaagtgttctgtatgcaatttgcgctgtgctctgctcctttgcagcaaggttcaatgcactcactgtctcccttgattccccgagcacacctacaccgtctgtgtgtctctatatggttacacataaatgtacaccacttgtgtacacgtgtatacacacgcccaaacattacttccagttcgctctggcctccaaaccttggcttgctgaaaac

SEQ ID NO: 28 - DNA, 106 bp, synthetic construct
  source 1..106: mol_type = other DNA; note = Description of Artificial Sequence: Synthetic polynucleotide
cggaaggaccgggctgaagcgtggccacgaggagggggatacccgtgcgagcgctgagccggcagagcggctgcagcccacgggctcctcggacccccgctgctgc

SEQ ID NO: 29 - DNA, 495 bp, synthetic construct
  source 1..495: mol_type = other DNA; note = Description of Artificial Sequence: Synthetic polynucleotide
cgtggagggggtggggtggaaagaagtagggaatggagaagattactaagaaaagtttcctgtctggaactgcggcagatctctttggatagagatgactacttaacctcactctgctttccttcgccgcggttgcggccgcgaccctgttcttaccaccagcaattcctccagggacttggtcagcagcccaacttgatctgcgtctctctgctaaggtgtttccgcaacagggtcaactccaagtctcacctttctaggaatcccgggcgcagcgcgggggtcgggactccgacctgtatttccaggcggaggtttccctgggtcaggcggccactctctgccagagattgtcagttatccaactgtcaatagagccgccgctccagcgagtttaatttaggcacagaaaagtcctgcctgggttgaggtgggcttaggatgagtttacttgagtgtgtgatttagaaatagatctatgggacagacagacac

SEQ ID NO: 30 - DNA, 237 bp, synthetic construct
  source 1..237: mol_type = other DNA; note = Description of Artificial Sequence: Synthetic polynucleotide
gcaccccccacctctgtgctttgcgcgctggtttcagattctctgaggaggcagcacaaccccaatccagtcctcagccctagtgaccctggagccggcgtgcagccaccagaaaaagattttcacatttcaaatcagtcttcagaagcccatccctccacaattgaacatcaagcaaatctcacaagccagcaagagccgctccaggccacttacattcaggctcccgctcctccg

SEQ ID NO: 31 - DNA, 459 bp, synthetic construct
  source 1..459: mol_type = other DNA; note = Description of Artificial Sequence: Synthetic polynucleotide
ggggttccagtctagcaggctgttctcacttggccccactccctccaccttttgtgttccagctccataatctgctccctgaggaaagggggctcgtcccttggggaagcacctccaactcccccatccccatttggtggcattctaagcaagcaacggcttcgggagagctgcctcgagagcctgagagaagtcccgcttagaagctgggctgggcaggtgcggagttgggggcgggaagccaggattgggcaagtggagctgcctgtgaccggcgccacagggcccagagcaagccgcttgctggttcaaccaggaaaccgaggtgcagaaggtggacgcagcgggccctggctcatagggtgcagggtcggtggcttacctgcaggcgctcccctccagaggtgccctcgcgtgggcttgctctgtgcgcctcggggacttcctcaaacggactcg

SEQ ID NO: 32 - DNA, 256 bp, synthetic construct
  source 1..256: mol_type = other DNA; note = Description of Artificial Sequence: Synthetic polynucleotide
gagacagagctttactatctcgctccctctcgcgcctccctcctcgctgggcattcaaacagctttccgacatcaccagccaaggatttttttccccgctctccttagtcgccgtccgtccatcagtacctgcaggggggaggaggaggagggaggaaagcggaaagaggaaaaagcataagcttgagccttccgatccgaccacgaatactcctgtaataaacccaccgccccaacaaatctgccatagcagccg

SEQ ID NO: 33 - DNA, 83 bp, synthetic construct
  source 1..83: mol_type = other DNA; note = Description of Artificial Sequence: Synthetic oligonucleotide
cggctttgaagaaggaaaaagtgagagcacaagcgagccagccaggagtcgaacctagaatcttctgatccgtagtcagacgc

SEQ ID NO: 34 - DNA, 47 bp, synthetic construct
  source 1..47: mol_type = other DNA; note = Description of Artificial Sequence: Synthetic oligonucleotide
cgcgacggccccaattccagcaacgctagagggcgcccgtgccaagc

SEQ ID NO: 35 - DNA, 214 bp, synthetic construct
  source 1..214: mol_type = other DNA; note = Description of Artificial Sequence: Synthetic polynucleotide
cgctggaatgtggcaaaaacaaaacactccccacccatgcacatcaccatctcctgactgcgggctggggggaagggagttcagggccaatgtgtcccagacttcagcgttccccacggctgtgtcagggctggggtggcttatccccctacagacgaaaatcaagatttaaaagcatactcttactgtggtttctctatcaaagcccatcaac

SEQ ID NO: 36 - DNA, 817 bp, synthetic construct
  source 1..817: mol_type = other DNA; note = Description of Artificial Sequence: Synthetic polynucleotide
gcataaattaacaacttctcagcctcggtttcccaacctgtgcaatgcagctcatattactttgcccacttgagccaggtgatctcttgaagagtaggaaataatgttggactggagttggaattctggttccaaaaggaggggattggggggcatttgggtctcctcctacctttctgcaagctctgaaaatcgtccatctcctcaggagaagcccacagaacaaacgatttcccaagcatcagaggagggcagacataacataacagagcagggaatcgggttagaacgggttatgctctgattttttggaaaatgggcaaacggggcggggactagggaggattccgccagccgggagttgggaggccgcgcgcgcctctgcggagcgtgacggccacggtcgcctagcaacgagcggggatgcgcggggcgcctgggtgggcgaggggtgctcgccgccgccgccgcgcgccctcgggcccccgggaccgcgggaaaacttcgcggctcctcggcgctggggcgtcggcgccaggcccggcagacagagcaatgcgccccgcgggctgagggcagaggatgcggccgcggcccagcgcccggccgggggagccgcggggtggcacgcggggaaagttggcgcgcgcctgaccgcgcctggaagccgcgcggtgccagggccgagttgtcccccaagtttctgcggcgatttgtcactccctggggatctggcggtctgaatcccgcggggcctccggctcagggattctgagcgctgggagagagaagcccgcgcttttcccggggacctgcg

SEQ ID NO: 37 - DNA, 743 bp, synthetic construct
  source 1..743: mol_type = other DNA; note = Description of Artificial Sequence: Synthetic polynucleotide
gggctgctcaaaccgggcagctgaagtccttagtgacctcagatcgtcaagtcaaaacgctgaatttccaccagcctctgtctgctttttgccaaataactggtggatggatgaaaagcattttgcagatatcttagaacatcacagtttcgatacgttgaggaattactattttcttatgattttcaagctgtagaagtgagggtttttacttacactgaaatgaacacatttaaataaatttgagcattggcaaagggggaaaaaaagaggcgcaaattaccacgctcattatatagaaggagctttttcagttcagagccagacattccctttgctgagtctaagttagaatctgtggtgaattataagcctacttttctatccttgttacttcttccttcttttccagaactccttaatttgttaatcaatgaatagagagcgactgtccccacagctccttaagtttcttaactctccttctcctttgtctactgttatttcattctttttaattaattgataaggatcagcttcgcttttttttttccctccccaaatctcagggaattcaactttttaaaaggtttatagtatggatcacttcttctaggaactttttctcctttaatctgggctttttcaaacggtatcttttaagcacacaagatatttcccaacatgatttcagataagatgtctcaaagaagaaaatagttaggtatttttaaattgcttcg

SEQ ID NO: 38 - DNA, 38 bp, synthetic construct
  source 1..38: mol_type = other DNA; note = Description of Artificial Sequence: Synthetic oligonucleotide
gcctgccgacttagggctcccggagctcgccggccgcg

SEQ ID NO: 39 - DNA, 473 bp, synthetic construct
  source 1..473: mol_type = other DNA; note = Description of Artificial Sequence: Synthetic polynucleotide
gttttcatacgggaactggatggaatgactccccaaaaaatacagctttatttctcaaatactgacccccaaagcactatctagtaatatatttgattgatctttcaaagtcagtaaaccacaaaggtttgtgtaatggcttgtacttaacgcctgatacctgagtaaagtttgaagcattaacataggaacagttcacttggaacaaaaatttattttctgaatgacctataaaggttgtcagagaaggtcttagtacatgattgaataacttggtctaacttacaggaagaataaagggcatttattttaaacctgtgtgttccccattctataatgggctgcctaggcattaaaggctcttgacatacttaagctcttgactgagggcgtgttggagattaccccttgtttttgagtacaatagttttgtttgctttttttcttttttaagtaacatttgtttttagacg

SEQ ID NO: 40 - DNA, 103 bp, synthetic construct
  source 1..103: mol_type = other DNA; note = Description of Artificial Sequence: Synthetic polynucleotide
cgatcccgaacccctgtagctaaagcggattgagcgcacccccgatgccctcgaccttatctggatacatttcttgcttcagaaacttcctctcatgacccac

SEQ ID NO: 41 - DNA, 70 bp, synthetic construct
  source 1..70: mol_type = other DNA; note = Description of Artificial Sequence: Synthetic oligonucleotide
cgaaatcggaataaaacgatgttattgagagagggctaaatcccagagtaaatttcaaacgaaataaatc

SEQ ID NO: 42 - DNA, 191 bp, synthetic construct
  source 1..191: mol_type = other DNA; note = Description of Artificial Sequence: Synthetic polynucleotide
cgcggggtgcagcatgagtcttcctttgtggcgtgcggctccatcggaacgcgcgttgcgacgacaaattccttttttcccccccgcagttaacagttctggggcagaggctggtggagaggtccagagcccactcagaccgagatgaagatgaggaaaagcatgagcaggaagaggctggcggctgcggc

SEQ ID NO: 43 - DNA, 261 bp, synthetic construct
  source 1..261: mol_type = other DNA; note = Description of Artificial Sequence: Synthetic polynucleotide
cggagccgagtgctcggtctctttccccgggacgggacagggagtggagttcctagccccttgggtgggaaaagccccgcgcacgtcaccgggtcccgtctgctcactgcttctgcatattttaagcctggacacagcctcctttaggatagaaaggcatttcccaaacaacaccgattctgggggtgtagtgggcctggcgctgggtcctggagagaaggttcagccccccttctcatccctgtactttggggatggctc

SEQ ID NO: 44 - DNA, 40 bp, synthetic construct
  source 1..40: mol_type = other DNA; note = Description of Artificial Sequence: Synthetic oligonucleotide
ggcgccggcgcggccaccgggagggcagagccagccgccg

SEQ ID NO: 45 - DNA, 161 bp, synthetic construct
  source 1..161: mol_type = other DNA; note = Description of Artificial Sequence: Synthetic polynucleotide
ggtgcctatctgctcgaatccatcccaaagattttcttctcgatacacttgggttttagtggtgggagtctgagacctagggagggatccccgggtggcctattgtgagaaatatgagactacatgtgcatctcctggaaaagcactttgcacagcgcccg

SEQ ID NO: 46 - DNA, 159 bp, synthetic construct
  source 1..159: mol_type = other DNA; note = Description of Artificial Sequence: Synthetic polynucleotide
gtttgcgagttgctgggctgcggtcgtgggtgggactcgccgcagaagcaagtgccagtggcccggcgggggtctcctcactcgcgctcgctccgactagcggcggagggactgcggcaggacgcgagctgagcccggccaaggccgctgcgctcagcg

SEQ ID NO: 47 - DNA, 362 bp, synthetic construct
  source 1..362: mol_type = other DNA; note = Description of Artificial Sequence: Synthetic polynucleotide
gtcggtgatgccctgagcagaagctgtggggctgatttaagtgtgtgctctgacagctcccgggctgctccggcgcttatctcttctaatcttactttcattgacttaaaacagctgccggtttgccaggggaaaaaaatcctattaaatctcaaaccagggtggggcgggggcgggggttcctgacagtgatcttcccagagctagtgtccagaaaagagcagggggaagggagagcattcacgggggtggctgcgtgttgggaagggggtgatggaaaggggactaaggaaacaattcgagcaacacgtttcttttcctcgctgtagccttctctccttttgcttttatgccgaggtgcg

SEQ ID NO: 48 - DNA, 43 bp, synthetic construct
  source 1..43: mol_type = other DNA; note = Description of Artificial Sequence: Synthetic oligonucleotide
cggcgacaggggaatggggcgaggcggcgcaggactccactgc

SEQ ID NO: 49 - DNA, 27 bp, synthetic construct
  source 1..27: mol_type = other DNA; note = Description of Artificial Sequence: Synthetic oligonucleotide
cgaatattcccagtcgcccgtggcgac

SEQ ID NO: 50 - DNA, 587 bp, synthetic construct
  source 1..587: mol_type = other DNA; note = Description of Artificial Sequence: Synthetic polynucleotide
cgcagagagagagaaaggaggaaggcctgcagctctagacttagcacctgtgaacttagttggaaataactcctgacagacatcaaccataacaccttctaacagaaacatgctgataggcaggatgttcaaacggggcagaagagggagcaggcaaccctgtgaaagtaacagcagcagaaacaaacacaccattccttctccccgcaacccacttggagcccctcctcagatcgcccctcccaacccctctgctctggccacgatcgcacctggcctcccggtccccctaacttccctcccacctctcccgcagcctgcgcctgagcctgagccaggtcgcggagtttgagactgacgcaaaggaggcacccccgcagcagagatgctcgtctttctgccacacaccctggaggacccgacagactggcagcagaaactaaagactgttcctgccgtcctctttccaacctctgcatgccccaagaggggtctgaccccggaatcctggagtccagggtgccccgccggggcgcaggaaggaaactcaggtagctgacaagttcaggcggccgtccttctccagc

SEQ ID NO: 51 - DNA, 78 bp, synthetic construct
  source 1..78: mol_type = other DNA; note = Description of Artificial Sequence: Synthetic oligonucleotide
gccaggaaccgcaggcgtggggacccaaacgtcacccgtggcctgatcctagataagaagtcccttgaaggcctgtcg

SEQ ID NO: 52 - DNA, 57 bp, synthetic construct
  source 1..57: mol_type = other DNA; note = Description of Artificial Sequence: Synthetic oligonucleotide
ggtggaaagaatcgatttcaaaattcaagctcaccgctgctcaacaaggcgcgcacg

SEQ ID NO: 53 - DNA, 728 bp, synthetic construct
  source 1..728: mol_type = other DNA; note = Description of Artificial Sequence: Synthetic polynucleotide
cgccccgttgccagggagagtgaatacagctagggacgtgaagggtaggtctgggctgggcattgaggagggtattaggctaggaagtatacctcacctttgtccaaatcgggtggttcggcctcctctcattggccctatgcagctgggttgcctctctcacccgcacccaagggtctttccagagtgttgcgtcatttccagcccagggagctgccttctttcctaaactgcaatggaaactgtcctgatgtctgagacaatgtccgttgtgccgcagccctctgccttctctagccagagcgcgagctcagctgcttttgagagaaatcagccacctggcccacctgtgcacaacttcagagctttgtagggggtgacaaggactgtgtcttcagggaaatgtcacgggcattagcgcctttttcgcaaatgtgaaagttgagaaatcaagaaggttaattattgggttgcccaagatctgctaagagcaaaggagaaaactccgtttcccaggcatgtgtcttgtgagccatttttaaatcaaccctcttaagtggacaagctccagaacacaacatgaagctgatgatgacttaggcaatttatgcttgaactcattggcctcatctcaagtcagtgtctcagagacacaggtgggacctgatccccaaggaacagatagcattccagattcatgggagcaacttttgagatgtggagcaccc

SEQ ID NO: 54 - DNA, 75 bp, synthetic construct
  source 1..75: mol_type = other DNA; note = Description of Artificial Sequence: Synthetic oligonucleotide
ggtcacatacgctaacaagacacggtgaaaagtctcttctcatcggcttggtgtgctgctctcttctctctctcg